SEQUENCE LISTING (51 sequences)

SEQ ID NO: 1 - DNA, 646 bp, Artificial Sequence
  engineered sequence
atgagcaaaggagaagaacttttcactggagttgtcccaattcttgttgaattagatggtgatgttaatgggcacaaattttctgtcagaggagagggtgaaggtgatgctacaaacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaaaggtattgattttaaagaagatggaaacattctcggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagctaa

SEQ ID NO: 2 - protein, 214 aa, Artificial Sequence
  engineered sequence
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEK

SEQ ID NO: 3 - DNA, 651 bp, Artificial Sequence
  Engineered Sequence
atgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcgacacaaactgtcctttcgaaagatcccaacgaaaagggtacctaa

SEQ ID NO: 4 - protein, 216 aa, Artificial Sequence
  Engineered Sequence
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGT

SEQ ID NO: 5 - DNA, 651 bp, Artificial Sequence
  Engineered Sequence
atgagcaaaggagaagaacttttcactggagttgtcccaattcttgttgaattagatggagatgttaatgggcacaaattttctgtcagaggagagggtgaaggtgatgctacaaacggaaaactcacccttaaattcatttgcactactggaaaactacctgttccatggccaacgcttgtcactactctgacctatggtgttcaatgcttttcccgttatccggatcacatgaaacagcatgactttttcaagagtgccatgcccgaaggttatgtacaggaacgcactatatatttcaaagatgacgggaactacaagacgcgtgctgtagtcaagtttgaaggtgatacccttgttaatcgtatcgagttaaagggtactgattttaaagaagatggaaacattctcggacacaaactcgagtacaactttaactcacacaatgtatatatcacggcagacaaacaaaagaatggaatcaaagctaacttcacaattcgccacaacgttgtagatggttccgttcaactagcagaccattatcaacaaaatactccaattggcgatggccctgtccttttaccagacaaccattacttgtcgacacaaactgtcctttcgaaagatcccaacgaaaagggtacctaa

SEQ ID NO: 6 - protein, 216 aa, Artificial Sequence
  Engineered Sequence
MSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIYFKDDGNYKTRAVVKFEGDTLVNRIELKGTDFKEDGNILGHKLEYNFNSHNVYITADKQKNGIKANFTIRHNVVDGSVQLADHYQQNTPIGDGPVLLPDNHYLSTQTVLSKDPNEKGT

SEQ ID NO: 7 - DNA, 84 bp, Artificial Sequence
  Engineered Sequence
aagcgtgaccacatggtccttcttgagtttgtaactgctgctgggattacacatggcatggatgagctctacaaaggtacctaa

SEQ ID NO: 8 - protein, 27 aa, Artificial Sequence
  Engineered Sequence
KRDHMVLLEFVTAAGITHGMDELYKGT

SEQ ID NO: 9 - DNA, 60 bp, Artificial Sequence
  Engineered Sequence
aagcgtgaccacatggtccttcttgagtttgtaactgctgctgggattacaggtacctaa

SEQ ID NO: 10 - protein, 19 aa, Artificial Sequence
  Engineered Sequence
KRDHMVLLEFVTAAGITGT

SEQ ID NO: 11 - DNA, 60 bp, Artificial Sequence
  Engineered Sequence
aagcgtgaccacatggtccttcatgagtttgtaactgctgctgggattacaggtacctaa

SEQ ID NO: 12 - protein, 19 aa, Artificial Sequence
  Engineered Sequence
KRDHMVLHEFVTAAGITGT

SEQ ID NO: 13 - DNA, 54 bp, Artificial Sequence
  Engineered Sequence
aagcgtgaccacatggtccttcatgagtctgtaaatgctgctgggggtacctaa

SEQ ID NO: 14 - protein, 17 aa, Artificial Sequence
  Engineered Sequence
KRDHMVLHESVNAAGGT

SEQ ID NO: 15 - DNA, 51 bp, Artificial Sequence
  Engineered Sequence
cgtgaccacatggtccttcatgagtctgtaaatgctgctgggattacataa

SEQ ID NO: 16 - protein, 16 aa, Artificial Sequence
  Engineered Sequence
RDHMVLHEYVNAAGIT

SEQ ID NO: 17 - DNA, 596 bp, Artificial Sequence
  Engineered Sequence
atgcgcaaaggagaagaacttttcactggagttgtcccaattcttgttgaattagatggtgatgttaatgggcacaaattttctgtccgtggagagggtgaaggtgatgctacaaacggaaaactcagccttaaatttatttgcactactggaaaactacctgttccatggccaacacttgtcactactctgacctatggtgttcaatgcttttcccgttatccggatcacatgaaacggcatgactttttcaagagtgtcatgcccgaaggttatgtacaggaacgcactatatctttcaaagatgacgggacctacaagacgcgtgctgaagtcaagtctgaaggtgatacccttgttaatcgtatcgagttaaaaggtattgattttaaagaagatggaaacattctcggacacaaactcgagtacaactttaactcacacaatgtatacatcacggcagacaaacaaaagaatggaatcaaagctaacttcacaattcgccacaacgttgaagatggttccgttcaactagcagaccattatcaacaaaatactccaattggcgatggccctgtccttttaccagacaataa

SEQ ID NO: 18 - protein, 196 aa, Artificial Sequence
  Engineered Sequence
MRKGEELFTGWPILVELDGDVNGHKFSVRGEGEGDATNGKLSLKFICTTGKLPVPWPTLVTTLTYGVQCFSRYPDHMKRHDFFKSVMPEGYVQERTISFKDDGTYKTRAEVKSEGDTLVNRIELKGIDFKEDGNILGHKLEYNFNSHNVYITADKQKNGIKANFTIRHNVEDGSVQLADHYQQNTPIGDGPVLLPD

SEQ ID NO: 19 - protein, 41 aa, Artificial Sequence
  Engineered Sequence
DHYLSTQTILSKDPNEERDHMVLLESVTAAGITHGMDELYK

SEQ ID NO: 20 - DNA, 39 bp, Artificial Sequence
  Engineered Sequence
agatatacatatgcgttttgccatcgtggtgaccgggcc

SEQ ID NO: 21 - DNA, 37 bp, Artificial Sequence
  Engineered Sequence
aattcggatccccaggccatctggctggagtgcttaa

SEQ ID NO: 22 - DNA, 39 bp, Artificial Sequence
  Engineered Sequence
agatatacatatgaaacgaattgcgtttgttttttctac

SEQ ID NO: 23 - DNA, 37 bp, Artificial Sequence
  Engineered Sequence
aattcggatccaaataacatcgtagttggcgagttcg

SEQ ID NO: 24 - DNA, 39 bp, Artificial Sequence
  Engineered Sequence
agatatacatatgtcttttgtgatcacaaatcccgaggc

SEQ ID NO: 25 - DNA, 37 bp, Artificial Sequence
  Engineered Sequence
aattcggatccagtgtctgtacgcgatgacgccgtgc

SEQ ID NO: 26 - DNA, 39 bp, Artificial Sequence
  Engineered Sequence
agatatacatatgcatttcgaagcgtacccaccggaggt

SEQ ID NO: 27 - DNA, 39 bp, Artificial Sequence
  Engineered Sequence
agatatacatatggacgcggcattggcctgcaccgtgct

SEQ ID NO: 28 - DNA, 37 bp, Artificial Sequence
  Engineered Sequence
aattcggatccccaggcttgtgttacaccctggccag

SEQ ID NO: 29 - DNA, 39 bp, Artificial Sequence
  Engineered Sequence
agatatacatatgcaacgagcgcgttgttatctgatagg

SEQ ID NO: 30 - DNA, 37 bp, Artificial Sequence
  Engineered Sequence
aattcggatccattttcattgtgcagccgccacgcta

SEQ ID NO: 31 - DNA, 39 bp, Artificial Sequence
  Engineered Sequence
agatatacatatgaccaccccacctcccaggatgctgcc

SEQ ID NO: 32 - DNA, 37 bp, Artificial Sequence
  Engineered Sequence
aattcggatccctccgccgccagcgcgtcgattgccg

SEQ ID NO: 33 - DNA, 39 bp, Artificial Sequence
  Engineered Sequence
agatatacatatgaccctgggcaccgtccacaattacgg

SEQ ID NO: 34 - DNA, 37 bp, Artificial Sequence
  Engineered Sequence
aattcggatccggtctcgaacggtcgccgggggtagg

SEQ ID NO: 35 - DNA, 36 bp, Artificial Sequence
  Engineered Sequence
tagagatactgagcacatcagcaggacgcactgacc

SEQ ID NO: 36 - DNA, 30 bp, Artificial Sequence
  Engineered Sequence
gaggcctctagaggttatgctagttattgc

SEQ ID NO: 37 - protein, 724 aa, Artificial Sequence
  engineered sequence
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 38 - protein, 235 aa, Artificial Sequence
  engineered sequence
MGSSHHHHHHSSGLVPRGSHMSXXYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLFDCDTPSVDLEMIDVHVLADAFKRYLPSTSGSDGGSGGGSTSRDHMVLHEYVNAAGIT

SEQ ID NO: 39 - protein, 147 aa, Artificial Sequence
  engineered sequence
MGSSHHHHHHSSGLVPRGSHMSGPKPRPPRPLPVAPGSSKTEADVEQQALTLPDLAEQFAPPDIAPPLLIKLVEAIEKKGLECSTLYRTQSSSNLAELRQLLDCDTPSVDLEMIDASTSGSDGGSGGGSTSRDHMVLHEYVNAAGIT

SEQ ID NO: 40 - protein, 279 aa, Artificial Sequence
  engineered sequence
MGSSHHHHHHSSGLVPRGSHMSGAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGTSTSGSDGGSGGGSTSRDHMVLHEYVNAAGIT

SEQ ID NO: 41 - protein, 207 aa, Artificial Sequence
  engineered sequence
MGSSHHHHHHSSGLVPRGSHMSGAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKPSTSGSDGGSGGGSTSRDHMVLHEYVNAAGIT

SEQ ID NO: 42 - protein, 246 aa, Artificial Sequence
  engineered sequence
MGSSHHHHHHSSGLVPRGSHMSGEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNASTSGSDGGSGGGSTSRDHMVLHEYVNAAGIT

SEQ ID NO: 43 - protein, 208 aa, Artificial Sequence
  engineered sequence
MGSSHHHHHHSSGLVPRGSHMSXXEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQDAEWYWGDISREEVNEKLRDTADGTFLASTSGSDGGSGGGSTSRDHMVLHEYVNAAGIT

SEQ ID NO: 44 - DNA, 715 bp, Artificial Sequence
  engineered sequence
atgggcagcagccatcatcatcatcatcacagcagcggcctggtgccgcgcggcagccatatgtcaggtgtatgattataaaaaggaaagagaagaagatattgacttgcacttgggtgacatattgactgtgaataaagggtccttagtagctcttggattcagtgatggacaggaagccaggcctgaagaaattggctggttaaatggctataatgaaaccacaggggaaaggggggactttccgggaacttatgtagaatatattggaaggaaaaaaatctcgcctcccacaccaaagccccggccacctcggcctcttcctgttgcaccaggttcttcgaaaactgaagcagatgttgaacaacaagctttgactctcccggatcttgcagagcagtttgcccctcctgacattgccccgcctcttcttatcaagctcgtggaagccattgaaaagaaaggtctggaatgttcaactctatacagaacacagagctccagcaacctggcagaattacgacagctttttgattgtgatacaccctccgtggacttggaaatgatcgatgtgcacgttttggctgacgctttcaaacgctatctcccctctactagtggatccgatggagggtctggtggcggatcaacaagccgtgaccacatggtccttcatgagtacgtaaatgctgctgggattacataaggtacctaa

SEQ ID NO: 45 - protein, 24 aa, Artificial Sequence
  engineered sequence
MGSSHHHHHHSSGLVPRGSHMSGV

SEQ ID NO: 46 - protein, 234 aa, Artificial Sequence
  engineered sequence
WAAAIIIIITAAAWCRAAAICQVYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLFDCDTPSVDLEMIDVHVLADAFKRYLPSTSGSDGGSGGGSTSRDHMVLHEYVNAAGIT

SEQ ID NO: 47 - protein, 41 aa, Artificial Sequence
  engineered sequence
GQQPSSSSSQQRPGAARQPYVRCMIIKRKEKKILTCTWVTY

SEQ ID NO: 48 - DNA, 634 bp, Artificial Sequence
  engineered sequence
atgggcagcagccatcatcatcatcatcacagcagcggcctggtgccgcgcggcagccatatgtcagggtgaaatgatttctttagctccagaagtacaaagctccgaagaatatattcagctattgaagaagcttattaggtcgcctagcatacctcatcagtattggcttacgcttcagtatttgttaaaacatttcttcaagctctctcaaacctccagcaaaaatctgttgaatgcaagagtactctctgaaattttcagccctatgcttttcagattctcagcagccagctctgataatactgaaaacctcataaaagttatagaaattttaatctcaactgaatggaatgaacgacagcctgcaccagcactgcctcctaaaccaccaaaacctactactgtagccaacaacggtatgaataacaatatgtccttacaagatgctgaatggtactggggagatatctcgagggaagaagtgaatgaaaaacttcgagatacagcagacgggacctttttggcctctactagtggatccgatggagggtctggtggcggatcaacaagccgtgaccacatggtccttcatgagtacgtaaatgctgctgggattacataaggtacctaa

SEQ ID NO: 49 - protein, 23 aa, Artificial Sequence
  engineered sequence
MGSSHHHHHHSSGLVPRGSHMSG

SEQ ID NO: 50 - protein, 207 aa, Artificial Sequence
  engineered sequence
WAAAIIIIITAAAWCRAAAICQGEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQDAEWYWGDISREEVNEKLRDTADGTFLASTSGSDGGSGGGSTSRDHMVLHEYVNAAGIT

SEQ ID NO: 51 - protein, 24 aa, Artificial Sequence
  engineered sequence
GQQPSSSSSQQRPGAARQPYVRVK

(19) United States
(12) Patent Application Publication
(10) Pub. No.: US 2014/0024555 A1
(43) Pub. Date: Jan. 23, 2014

(54) METHOD OF IDENTIFYING SOLUBLE PROTEINS AND SOLUBLE PROTEIN COMPLEXES

(71) Applicants: Geoffrey S. Waldo, Santa Fe, NM (US); Meghan A. Lockard, New York, NY (US)

(72) Inventors: Geoffrey S. Waldo, Santa Fe, NM (US); Meghan A. Lockard, New York, NY (US)

(73) Assignee: LOS ALAMOS NATIONAL SECURITY, LLC, Los Alamos, NM (US)

(21) Appl. No.: 14/008,474

(22) PCT Filed: Mar. 29, 2012
(86) PCT No.: PCT/US12/31281

§ 371 Date: Sep. 27, 2013

Related U.S. Application Data
(60) Provisional application No. 61/469,938, filed on Mar. 31, 2011.

(51) Int. Cl. G01N 33/544

(52) U.S. Cl. CPC G01N 33/544
USPC 506/9; 506/14

(57) ABSTRACT

Provided herein are methods of identifying a protein as soluble, as well as methods of identifying a soluble protein complex of at least two proteins. The methods allow for the determination of in vitro solubility, or both in vitro and in vivo solubility, of a protein or protein complex.

### CROSS REFERENCE TO RELATED APPLICATIONS

This application claims the benefit of U.S. Provisional Application No. 61/469,938, filed Mar. 31, 2011, which is herein incorporated by reference in its entirety.

### STATEMENT OF GOVERNMENT SUPPORT

This invention was made with government support under Contract No. DE-AC52-06NA25396 awarded by the U.S. Department of Energy, and under the National Institutes of Health's Protein Structure Initiative, grant number 5U54GM074946-4. The government has certain rights in the invention.

### FIELD

Methods for the determination of in vitro solubility, or both in vitro and in vivo solubility, of a test protein, in one simple assay, are disclosed herein. Further disclosed is a method of identifying a soluble protein complex of at least two proteins.

### BACKGROUND

A common problem in biology is the misfolding and aggregation of proteins expressed in heterologous hosts. One solution to this problem is to screen libraries of mutant proteins or protein domains for more soluble or stable forms. Existing screens each have virtues and limitations. What one method gives in reliable in vitro solubility data, it lacks in throughput (Knaust and Nordlund, Anal. Biochem., 297:79-85, 2001). Others may have higher throughput, but they require unique and often expensive equipment (Tarendeau et al., Nat. Struct. Mol. Biol., 14:229-233, 2007; Ramachandran et al., Nat. Methods, 5:535-538, 2008) or suffer from false positive results and labor intensive protocols (see, e.g., Cabantous and Waldo, Nat. Methods, 3:845-854, 2006; Cornvik et al., Nat. Methods, 2:507-509, 2005; U.S. Pat. No. 7,718,381).

Split-fluorescent proteins (SFPs) are composed of multiple peptide fragments that individually are not fluorescent, but, when complemented, form a functional fluorescent molecule. For example, Split-Green Fluorescent Protein (GFP) is a SFP. Some engineered split-GFP molecules are self-assembling. (See, e.g., U.S. Pat. App. Pub. No. 2005/0221343 and PCT Pub. No. WO/2005/074436; Cabantous et al., Nat. Biotechnol., 23:102-107, 2005; Cabantous and Waldo, Nat. Methods, 3:845-854, 2006.)

### SUMMARY

Described herein are methods of identifying in vitro solubility, or both in vitro and in vivo solubility (e.g., total protein expression in vivo), of a test protein in one simple assay. These methods include use of an affinity reagent immobilized in a hydrogel in combination with a colony-based soluble SFP assay, which allows high throughput screening for soluble protein and reduces (or eliminates, in some embodiments) false positive results. Described methods can be used to identify soluble protein complexes.

A method of identifying a soluble protein is provided. In some embodiments, the method includes expressing within at least one host cell a first heterologous amino acid molecule including a first test protein; bringing the at least one host cell into aqueous contact with the surface of a hydrogel including an immobilized affinity reagent with affinity for the first heterologous amino acid molecule for a period of time sufficient for transfer of the first amino acid molecule into the hydrogel; and detecting a complex of the first heterologous amino acid molecule and the immobilized affinity reagent in the hydrogel, wherein the presence of a complex of the first amino acid molecule and the immobilized affinity reagent in the hydrogel identifies the first test protein as a soluble protein.

In some embodiments of the method of identifying a soluble protein, the first heterologous amino acid molecule includes a detection tag that does not bind the immobilized affinity reagent and detecting the complex of the first heterologous amino acid molecule and the immobilized affinity reagent in the hydrogel includes detecting the presence of the detection tag immobilized in the hydrogel. In some embodiments, the detection tag is a Split Fluorescent Protein (SFP) tag, the hydrogel includes a SFP detector, and detecting the presence of the detection tag immobilized in the hydrogel includes detecting immobilized complemented Split Fluorescent Protein fluorescence in the hydrogel.

Also provided are kits for performing the methods described herein. In some embodiments, a kit includes a nucleic acid construct encoding a SFP tag and a multiple cloning site adjacent thereto, such that an encoding sequence inserted into the multiple cloning site results in a nucleic acid molecule that encodes a test protein fused with the SFP tag; a hydrogel including an immobilized affinity reagent; and instructions for carrying out the method.

Also provided are systems for detecting a soluble protein. In some embodiments, a system includes a first nucleic acid construct encoding a SFP tag and a multiple cloning site adjacent thereto, wherein insertion of a nucleic acid molecule encoding a test protein into the multiple cloning site is expressed as a heterologous amino acid nucleic acid molecule that encodes a heterologous amino acid including the test protein and the SFP tag; a second nucleic acid construct encoding a SFP detector or purified SFP detector, or both; a host cell including the first nucleic acid construct, the second nucleic acid construct, or both; and, a hydrogel including an immobilized affinity reagent with affinity for the first heterologous amino acid molecule.

It will be further understood that the methods of identifying a protein as soluble in vitro, or both in vitro and in vivo, as well as identifying a soluble protein complex in vitro, or both in vitro and in vivo, and the kits and systems, disclosed herein are useful beyond the specific circumstances that are described in detail herein. For instance, the methods are expected to be useful for any number of situations where it would be advantageous to identity a protein as soluble or to identify a soluble protein complex.

### BRIEF DESCRIPTION OF THE FIGURES

FIGS. 1A-1D illustrate development of a bead-based split-GFP colony filtration assay. (FIG. 1A) Comparison of liquid culture and plate based assays of 18 control proteins from P. aerophilum (Table 1). Image OA sec exposure, 488 nm excitation) of microtiter plate wells (top) containing soluble fraction (first row), insoluble fraction (second row) and Talon® metal affinity resin-bound soluble fraction (third row) for control proteins expressed in 1 mL LB liquid culture (350 RPM, 37° C. for 4 h) after co-induction. Image of E. coli colonies expressing tagged proteins co-expressed with the GFP 1-10 detector at 37° C. for 4 h, reflecting total expression (½ sec exposure) (fourth row). Application of the split-GFP colony filtration immobilized bead assay (bottom row) outlined in FIG. 1B. Image of capture plate showing bound fluorescence after partial colony lysis. The image reflects soluble, bead-bound protein (2 sec exposure). (FIG. 1B) Principle of bead-binding assay for soluble proteins. A protein of interest is flanked by a 6-His tag on its N-terminus and the GFP S11 tag (β-strand 11, residues 215-230) on its C-terminus via a flexible linker (L). The complementary GFP 1-10 detector (β-strands 1-10, residues 1-214) is co-expressed separately in the same cell (top). The GFP S11 tag rapidly associates with the abundant GFP 1-10, committing the GFP to fold and form the fluorophore, whether the protein of interest subsequently remains soluble (left) or aggregates (right). The colonies may be imaged using a digital camera. Colonies resting on Durapore® membrane are partially lysed, soluble fluorescently-labeled proteins diffuse through the Durapore® membrane and bind to affinity beads immobilized in agarose (left). Insoluble aggregates cannot pass through the Durapore® membrane (right). Imaging the beads gives a measure of the soluble, bead-bound fluorescent protein. (FIG. 1C) Validation of the GFP colony filtration bead assay using 8 control multi-protein complexes (Table 2) tagged as in FIG. 1D. Image of colonies after co-expression (½ sec exposure) are shown in the top panel. Talon® metal affinity resin (Clontech, Mountain View, Calif.) capture plate 1-1/2 h after partial colony lysis and removal of colony membrane (middle), and after subsequent overnight incubation at 10° C. (4 s exposure) (bottom). Compact fluorescent spots indicate assembled soluble complexes. (FIG. 1D) Principle of bead-binding assay for complexes. A multi-protein complex carries a 6-His tag on one subunit, and the GFP S11 tag on another subunit for labeling with GFP fluorescence (top). After lysis and filtration for soluble proteins, bead-bound fluorescence indicates intact assembled complexes (top). Lack of bead-bound fluorescence indicates insoluble or unstable complexes, for example those missing a required subunit (bottom).

FIGS. 2A-2C illustrate representative colony selecting strategies for the Split Fluorescent protein colony filtration assay. (FIG. 2A) Overview of the split-GFP colony filtration assay applied to colony picking. Step 1: E. coli cells carrying plasmids encoding the tagged protein of interest and the GFP 1-10 detector (see FIG. 1A) are mixed with cells expressing red fluorescent protein bearing a 6-His tag, plated and grown overnight. Step 2: The colony membrane is moved to the induction plate then imaged. Colony fluorescence is proportional to total expression of the protein of interest. Red fluorescent clones will aid in later alignment of the image and colony membrane. Step 3: The colony membrane is moved to the capture plate containing metal affinity resin beads (Talon® metal affinity resin) immobilized in agarose. Colonies are partially lysed by misting with a chemical lysis reagent, releasing the protein of interest fused to fluorescent, complemented GFP. Soluble fluorescent proteins pass through the filtration membrane and bind to the beads. Insoluble proteins are retained on the membrane. Step 4: The colony membrane is returned to the LB agar plate for later picking, and the capture plate is imaged. The bead-bound fluorescence reflects soluble protein. Step 5: Image processing software is used to align the pictures of the fluorescent colonies (see Step 2) and the fluorescent bead capture plate (see Step 4). The images of desired colonies are highlighted, corresponding to brighter spots on the assay plate. Step 6: The marked colony membrane image is projected onto the colony membrane using a digital micro-projector, aligned using the red fluorescent clones, and the highlighted clones are picked. (FIG. 2B) Application to mock protein library included of soluble protein #17 (phosphate cyclase, Table 1) in a 25-fold excess of clones expressing insoluble protein #18 (purine-nucleoside phosphorylase, Table 1). Image of colonies (left), assay plate showing Talon® metal affinity resin-bound fluorescent, soluble protein (right). Aligned, superimposed images (middle) (¼ sec exposure). Clones expressing soluble protein #17, indicated with white arrows. Clones expressing red fluorescent protein aid in alignment of images. (FIG. 2C) Application to a mock library consisting of cells expressing YheNML (see FIG. 1C column 1 and Table 2) in a 25-fold excess of cells expressing unstable protein construct YheML (see FIG. 1C column 2). Image of colonies OA sec exposure) (left), capture plate 1½ h after lysis and transfer of colony membrane (middle), capture plate after additional overnight incubation at 4° C. (4 s exposure) (right). White arrows indicate clones expressing stable YheNML (left). Corresponding compact spots on assay plate (middle, right). Diffuse spots on capture plate corresponded to YheML.

FIG. 3 illustrates the effect of lysis cocktail and buffer composition of capture plate on yield of protein bound immobilized affinity beads. Lysis reagents were supplemented with 2 mM MgCl₂ and 10 AU/ml of Benzonase® nuclease (EMD chemicals, Gibbstown, N.J.). Column 1, BugBuster® Protein extraction reagent (EMD chemicals, Gibbstown, N.J.) lysis cocktail, TNG buffer (100 mM TRIS HCl pH 7.5, 10% v/v glycerol, 150 mM NaCl) in capture plate; Column 2, SoluLyse® protein extraction reagent (Genlantis, San Diego, Calif.) lysis, TNG in capture plate; Column 3, BugBuster® Protein extraction reagent (EMD chemicals, Gibbstown, N.J.) lysis, TRIS HCl pH 7.5 in capture plate; Column 4, SoluLyse® protein extraction reagent (Genlantis, San Diego, Calif.) lysis, TRIS HCl pH 7.5 in capture plate. Whole cell fluorescence after 4 hours co-induction (top); bead-bound fluorescence on the capture plate after 4 rounds of partial lysis as described herein.

FIGS. 4A-4B illustrate the effect of buffer on the stability of 8 control protein complexes. (FIG. 4A) Capture plates with 100 mM TRIS HCl, pH 7.5. Complexes 1-5 dissociate with time (4 columns, right). (FIG. 4B) Capture plates with TNG Buffer (100 mM TRIS HCl, pH 7.5, 10% v/v glycerol, 150 mM NaCl). Incomplete complexes 2 and 4 dissociate and/or fail to bind. Complete complexes (1, 3, 4-7) bind as expected.

FIG. 5 illustrates an example of preparation of a hydrogel including an affinity reagent linked to an agarose bead (i.e., preparation of a capture plate). Using a gloved finger, silicone vacuum grease is applied to the inner walls of two disposable 150 mm Bauer plates. The interior base of each plate was protected from grease using a protective disc. Approximately 20 ml of a molten agarose/Talon® metal affinity resin mixture is poured in the plate, allowed to solidify for ˜5 min, overlaid with ˜50 ml of molten agarose and the slab allowed to cool and solidify (20 min). The solidified agarose slabs were turned out of the plates, flipped over, laid back in the plate and seated by striking each plate on a paper towel stack. Any supernatant is discarded; the plates are dried for ˜1 hour and then stored for future use. Because the Talon® metal affinity resin sinks to the bottom of the plate when the molten agarose/Talon® metal affinity resin mixture initially added to the plate, flipping the agarose slab over results in an agarose slab containing Talon® metal affinity resin near its upper surface.

FIG. 6 illustrates an example of method steps required an embodiment of identifying a soluble protein or a soluble protein complex. Host cells are plated onto a Durapore® membrane (Millipore Co., Billerica, Mass.) resting on selective LB media. The host cells express (1) a test protein fused to an affinity tag and a SFP tag, and (2) a SFP detector, each protein expressed under the control of a different inducible promoter (or the host cells express appropriate proteins and protein fusion for detection of soluble protein complexes). After overnight incubation at 32° C., the Durapore® membrane carrying the colonies (now ˜1 mm diameter) is transferred face up to selective LB plates containing 350 ng/ml anhydrotetracycline (AnTET) and 1 mM isopropylthiogalactoside (IPTG) and incubated at 37° C. for 4 h to induce expression from the two inducible promoters (or incubated longer for expression of soluble protein complexes). Then the Durapore® membrane with the induced colonies is moved to a capture plate (e.g., as prepared according to FIG. 5) for further study.

FIG. 7 illustrates an example of imaging colonies expressing SFP as part of an in vivo screen for soluble protein, which screen is optionally included with the methods of detecting soluble protein and protein complexes in vitro described herein. Host cells expressing test protein-SFP tag fusions as well as the corresponding SFP detector are plated on a Durapore® membrane, expression is induced, and membrane is transferred to a plate for imaging as previously described (Cabantous et al., Nat. Biotechnol., 23:102-107, 2005) using an Illumatool Lighting System (Light Tools Research) to record fluorescence proportional to total protein expression.

FIG. 8 illustrates an example of partial chemical lysis of host cells to allow transfer of expressed proteins from the host cells to the hydrogel including the immobilized affinity reagent. A first population of host cells expressing both a test protein fused to a 6HIS and a GFP S11 tag and the corresponding GFP S 1-10 detector are plated on a Durapore® membrane, expression is induced, and membrane is transferred to a hydrogel that includes Talon® metal affinity resin. A second population of host cells is plated onto the same membrane, this second population expressing a 6HIS-dsRed fusion protein. The dsRed fluorescent protein can be differentially detected from the split-GFP fluorescent protein. The host cells on the membrane are misted with a cell lysis reagent. The treatment only results in partial lysis of the host cells, which release protein from the lysed cells. The membrane is allows to incubate on the hydrogel for approximately one hour, and is then moved to an LB plate for storage. If the 6HIS-test protein-GFP S11 fusion and the GFP S1-10 detector protein are capable of transiting from the cells into the hydrogel (e.g., if they are sufficiently soluble to pass through the membrane and into the hydrogel), then the Talon® metal affinity resin will bind to the 6HIS-test protein-GFP S11, which will for a functional fluorescent molecule with the GFP S1-10 detector. Additionally, the 6HIS-dsRed fusion protein released from the second population of host cells will transit in to the hydrogel and bind to the Talon® metal affinity resin. Differential detection of the complemented SFP and the fluorescent protein allows for orientation of the membrane to select host cells corresponding to any detected SFP fluorescence.

FIG. 9 illustrates an example of imaging immobilized SFP fluorescence in a hydrogel. The capture plate was imaged using the Illumatool system as previously described (Cabantous et al., Nat. Biotechnol., 23:102-107, 2005).

FIG. 10 illustrates projection of marked colony plate image onto colony plate for guided picking. Images of the colonies and capture plate were manually aligned based on the dsRed spots using the public domain software “HDR Alignment Plug-in” for ImageJ (Wayne Rasband, NIH). Using the aligned capture plate image, compact fluorescent spots were identified and the corresponding colonies for picking were highlighted in the colony image using Paint Shop Pro (Corel Corp., Ottawa, Ontario). The highlighted colony image was then displayed using Paint Shop Pro, and projected onto the original cell colonies using an MPro110 microprojector (3M, Minneapolis, Minn.; FIG. 10). Superposition of the image and target was optimized by adjusting the projection distance (ca. 30 cm) and magnification factor in Paint Shop Pro (ca. 34% full-scale setting) using the dsRed colonies as a guide.

FIG. 11 illustrates PCR and DNA gels to confirm identity of picks from a library consisting of cells expressing stable YheNML or unstable YheML (FIG. 1C columns 1 and 2). The image of colonies OA sec exposure) (left), capture plate 1½ h after lysis and transfer of colony membrane (middle), capture plate after additional overnight incubation at 4° C. (4 s exposure) (right) is shown. The image of PCR products amplified from DNA of picked clones (bottom). Picks with compact Talon® blots (colonies marked 1-7) give PCR products (lanes 1 to 7) with the same mass as YheNML control (lane 15). Picks from clones with diffuse Talon® blots (colonies marked 8-14) match the YheML control (lane 16).

FIG. 12 is a schematic representation of a colony-based split-GFP soluble protein screen where the complementing split-GFP S1-10 detector protein is added directly to the capture plate.

FIG. 13 illustrates results from a colony-based split-GFP soluble protein screen where the complementing split-GFP S1-10 detector protein is added directly to the capture plate.

FIG. 14 illustrates results from a colony-based split-GFP soluble protein screen where the complementing split-GFP S1-10 detector protein is added directly to the capture plate, which contains Talon® metal affinity resin. The bright speckles observed in the hydrogel in positions corresponding to a bacterial colony indicate the presence of affinity resin bound to 6HIS-Test Protein-S11:1-10 that has been complemented with the split-GFP S1-10 detector in the capture plate. Detection of diffuse GFP fluorescence indicates that the fluorescent signal is not immobilized the affinity resin, i.e., lacks the 6HIS-test protein-GFP S11/S1-10 fusion protein complex. Diffuse fluorescence in these images comes from the diffusion of GFP 1-10 up through the membrane and into the cell debris, staining the small amount of accessible S11 tag in the aggregates above the membrane. Thus, the detection of fluorescent speckles eliminates false positives. In this experiment, the immobilized complemented split-GFP was imaged directly through bacterial colony mass.

FIGS. 15A-15B illustrate the comparison of liquid culture and immobilized bead assays for total expressed protein and bead-bound soluble protein using GFP fluorescence. FIG. 15A is a graph illustrating co-induction fluorescence (proportional to total expression) of 18 control proteins listed in Table 1. Fluorescence was measured for single colonies (y-axis) or as liquid culture fluorescence (x-axis) (see Table 1 for tabulated values). The graph is a graphical representation of the corresponding fractions in the photographs shown FIG. 1A. FIG. 15B is a graph illustrating the comparison of Talon® bound protein in the colony-based immobilized bead assay (y-axis) and the Talon® bound in batch mode from liquid culture lysates (x-axis) of the 18 control proteins listed in Table 1 (see Table 1 for tabulated values). The graph is a graphical representation of the corresponding fractions shown in FIG. 1A.

FIGS. 16A-16B illustrate the results of the p85 large fragment library screen described in Example 2. FIG. 16A is a diagram of experimental scheme (top) and of the fragments containing at least half of the BCR domain tagged with Split-GFP S11 tag (enlarged inset bottom). FIG. 16B illustrates the results of scaled up expression of the 6 BCR hits (A-F) shown in FIG. 16A. Scanned images of the colonies (from the original Bauer plates of ˜4000 colonies per plate) corresponding to the BCR hits (¼ sec exposure) (row marked ‘colonies’) and Talon® capture plate (4 sec exposure) (row marked ‘Talon® bead blot’) are shown. Scanned image of SDS-PAGE gel of Talon® bound fractions of 3 ml liquid cultures run on a 4-20% SDS-PAGE and stained with Coomassie Blue (middle row) to visualize the over expressed proteins (indicated by arrows). The heavier protein in all the samples around 34 kDa is a contaminating E. coli protein. Talon® bound fluorescence after in vitro complementation with GFP 1-10 is also shown (bottom row).

FIG. 17 illustrates the effect of co-expression of Split GFP S11 tagged protein and GFP 1-10 detector fragment on protein solubility. The 18 control proteins listed in Table 1 were expressed as Split-GFP S11 tagged proteins from a pTET vector, and soluble and insoluble protein was assayed according to standard techniques (Cabantous et al., Nat. Biotech., 23:102-107, 2005; Cabantous and Waldo, Nat. Methods, 3:845-854, 2006) (x-axis). For co-induction (y-axis), tagged control proteins were co-expressed from the pTET plasmid along with the GFP 1-10 detector fragment (Cabantous et al., Nat. Biotech., 23:102-107, 2005; Cabantous and Waldo, Nat. Methods, 3:845-854, 2006). Fluorescence of protein in soluble and pellet fractions was measured by plate reader (Materials and Methods). Fraction soluble is defined as Fsol/(Fsol+Fpel), where Fsol and Fpel is the fluorescence of the soluble and pellet fractions, respectively.

FIG. 18 illustrates the effect of cell lysis buffer chemistry on protein solubility. The 18 control proteins listed in Table 1 were co-expressed as Split-GFP S11 tagged proteins from a pTET vector, along with the Split GFP 1-10 detector fragment and soluble and insoluble protein was assayed according to standard techniques (Cabantous et al., Nat. Biotech., 23:102-107, 2005; Cabantous and Waldo, Nat. Methods, 3:845-854, 2006) (x-axis). Fraction soluble is defined as Fsol/(Fsol+Fpel), where Fsol and Fpel is the fluorescence of the soluble and pellet fractions, respectively. Cells were lysed by sonication in TNG buffer (x-axis) or in SoluLyse supplemented with Benzonase and MgCl₂.

FIG. 19 illustrates an alignment of the p85 alpha (SEQ ID NO: 37) and BCR fragments (A-F; SEQ Id NOs: 38-43, respectively) isolated from the soluble protein screen described in Example 2 with p85 alpha reference sequence (top). The structural core of BCR corresponding to ordered residues in PDB entry 1PBW is shown (grey highlight). The Arrow indicates boundaries (amino acids 115-298), grey arrow from 299-309 is region seen in only one of the molecules in the unit cell (partial occupancy; Musacchio et al., Proc. Natl. Acad. Sci. U.S.A., 93:14373-14378, 1996) in PDB entry 1PBW. Fragments A and F each have a two base pair frame shift deletion just before the fragment. Since the co-expression (whole cell fluorescence) indicates the S11 tag is expressed, for these two (A and F) the frame has been arbitrarily restored at the lesion so that the downstream amino acid sequence is in the correct frame to account for the apparent shifting of the ribosome back into the frame of the GFP S11 tag. The N-terminal and C-terminal vector sequences are highlighted in light grey.

FIG. 20 illustrates the artifact for BCR Clone A (SEQ ID NO: 44, plus and minus strands are shown). The three-frame alternative open reading frame translation of BCR Clone A is shown (SEQ ID NOs: 45-47). Note frame shift (extra T base, in box) leading to stop codons in the 6HIS frame (bottom coding frame). Bases contributed by vector upstream (nucleotides highlighted in light grey) indicated near the extra T base (box). The downstream linker and GFP S11 tag (dark and medium grey highlight) is in the same frame as the BCR coding sequencing (middle coding frame). Observation of bead-bound GFP fluorescence (see FIG. 4B) indicates that the ribosome apparently reinitiates downstream of the extra base in the frame of the GFP S11 (light grey highlight of middle coding frame).

FIG. 21 illustrates the artifact for BCR Clone F (SEQ ID NO: 48, plus and minus strands are shown). The three-frame alternative open reading frame of BCR Clone A is shown (SEQ ID NOs: 49-51). Note frame shift leading to stop codon in the 6HIS frame (bottom coding frame). The downstream linker and GFP S11 tag (dark and medium grey highlight) is in the same frame as the BCR coding sequencing (middle coding frame). Bases upstream contributed by vector near frame shift (nucleotides highlighted in light grey). Extra (G) base from insert that results in frame shift (box) and resulting stop codon. Observation of cellular GFP fluorescence (see FIG. 4B) indicates that the ribosome apparently reinitiates downstream of the lesion in the frame of the GFP S11 (light grey highlight of middle coding frame).

### SEQUENCES

The nucleic and amino acid sequences provided herein are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜47 kb), which was created on Mar. 29, 2012, which is incorporated by reference herein. In the accompanying sequence listing:

SEQ ID NO: 1 is an exemplary nucleotide sequence encoding GFP superfolder 1-10.

SEQ ID NO: 2 is the amino acid sequence of GFP superfolder 1-10.

SEQ ID NO: 3 is an exemplary nucleotide sequence encoding GFP 1-10 OPT.

SEQ ID NO: 4 is the amino acid sequence of GFP 1-10 OPT (additional mutations compared to superfolder: N39I, T105K, E111V, 1128T, K166T, 1167V, 5205T).

SEQ ID NO: 5 is an exemplary nucleotide sequence encoding GFP 1-10 A4.

SEQ ID NO: 6 is the amino acid sequence of GFP 1-10 A4 (additional mutations compared to Superfolder GFP: R80Q, S99Y, T105N, E111V, 1128T, K166T, E172V, S205T).

SEQ ID NO: 7 is an exemplary nucleotide sequence encoding GFP S11 214-238.

SEQ ID NO: 8 is the amino acid sequence of GFP S11 214-238.

SEQ ID NO: 9 is an exemplary nucleotide sequence encoding GFP S11 214-230.

SEQ ID NO: 10 is the amino acid sequence of GFP S11 214-230.

SEQ ID NO: 11 is an exemplary nucleotide sequence encoding GFP S11 M1.

SEQ ID NO: 12 is the amino acid sequence of GFP S11 M1 (including the L221H substitution).

SEQ ID NO: 13 is an exemplary nucleotide sequence encoding GFP S11 M2.

SEQ ID NO: 14 is the amino acid sequence of GFP S11 M2 (Including the L221H, F2235 and T225N substitutions).

SEQ ID NO: 15 is an exemplary nucleotide sequence encoding GFP S11 M3.

SEQ ID NO: 16 is the amino acid sequence of GFP S11 M3 (including the L221H, F223Y and T225N substitutions).

SEQ ID NO: 17 is an exemplary nucleotide sequence encoding GFP 1-9 OPT.

SEQ ID NO: 18 is the amino sequence of GFP 1-9 OPT.

SEQ ID NO: 19 is the amino acid sequence of GFP 10-11 OPT.

SEQ ID NOs: 20-36 each show the nucleotide sequence of a DNA primer.

SEQ ID NO: 37 is the amino acid sequences of p85 alpha.

SEQ ID NOs: 38-43 are the experimentally tested BCR fragments A-F.

SEQ ID NO: 44 is the nucleic acid sequence of BCR Clone A.

SEQ ID NOs: 45-47 are the amino acid sequences of the alternative open reading frames of BCR Clone A.

SEQ ID NO: 44 is the nucleic acid sequence of BCR Clone F.

SEQ ID NOs: 45-47 are the amino acid sequences of the alternative open reading frames of BRC Clone F.

### DETAILED DESCRIPTION

### I. Terms and Abbreviations

cDNA Complementary DNA

CFP Cyan fluorescent protein

dsDNA Double-stranded DNA

DNA Deoxyribonucleic acid

DTT Dithiothreitol

ELISA Enzyme linked immunosorbant assay

GFP Green Fluorescent Protein

IPTG Isopropyl β-D-1-thiogalactopyranoside

LB agar Luria-Bertani agar

MCS Multiple cloning site

ORF Open reading frame

PBS Phosphate-buffered saline

PCR Polymerase chain reaction

PETE Polyethylene track-etched

RMSD Root mean square deviation

GFP S1-9 Beta strands 1-9 of GFP

GFP S1-10 Beta strands 1-10 of GFP

GFP S10 Beta strand 10 of GFP

GFP S11 Beta strand 11 of GFP

SDS-PAGE Sodium dodecyl sulfate-polyacrylamide gel electrophoresis

SFP Split Fluorescent Protein

Tet Tetracycline

TNG Tris-sodium-glycerol (buffer)

WB Western blot

YFP Yellow fluorescent protein

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid” includes single or plural nucleic acids and is considered equivalent to the phrase “comprising at least one nucleic acid.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to October 2010); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999; Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001; Chalfie and Kain (Eds), Green Fluorescent Protein: Properties, Applications and Protocols, First Edition, Wiley-Liss, 1998; or Hicks (ed.), Green Fluorescent Protein: Applications & Protocols, First Edition, Humana Press, 2001.

All publications, patent applications, patents, and other references mentioned herein are incorporated by reference in their entirety. All sequences referred to by GenBank Accession numbers herein are incorporated by reference as they appeared in the database on Dec. 10, 2010. In case of conflict, the present specification, including explanations of terms, will control. In addition, the materials, methods, and examples are illustrative only and not intended to be limiting.

Affinity Reagent: A molecule that is capable of binding to a ligand (e.g., affinity tags, detection tags, test proteins). Affinity reagents are well known to the skilled artisan. Examples include metal ions (e.g., copper, nickel, cobalt ions, among others), proteins (e.g., antibodies, streptavidin, GFP S1-10), small molecules (e.g., biotin) and peptides (e.g., glutathione). Typically, the affinity reagent is linked to a solid support, e.g., an agarose or other composition bead or surface, such as for instance a bead embedded in a hydrogel.

Agar: A heterogeneous mixture of two classes of polysaccharide: agaropectin and agarose. Agar, agaropectin and agarose are used for the manufacture of hydrogels.

Agent: Any substance or any combination of substances that is useful for achieving an end or result; for example, a substance or combination of substances useful for modulating gene expression or protein activity.

Amino acid: Naturally occurring or synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

Antisense and Sense: Double-stranded DNA (dsDNA) has two strands, a 5′->3′ strand, referred to as the plus strand, and a 3′->5′ strand (the reverse compliment), referred to as the minus strand. Because RNA polymerase adds nucleic acids in a 5′->3′ direction, the minus strand of the DNA serves as the template for the RNA during transcription. Thus, the RNA formed will have a sequence complementary to the minus strand and identical to the plus strand (except that U is substituted for T).

Binding: A specific interaction between two molecules. For example, binding can occur between a two fragments of a split fluorescent molecule (e.g., GFP S1-10 and GFP S11), or between a receptor and a particular ligand. Binding can be specific and selective, so that one molecule is bound preferentially when compared to another molecule. In one example, specific binding is identified by a disassociation constant (K_{d}) of an agent for a particular protein or class of proteins, compared to the K_{d} for one or more other cellular proteins. In another example, specific binding of an antagonist for a receptor is identified by an inhibitory concentration (IC₅₀).

cDNA (complementary DNA): A piece of DNA lacking internal, non-coding segments (introns) and transcriptional regulatory sequences. cDNA may also contain untranslated regions (UTRs) that are involved in translational control in the corresponding RNA molecule. cDNA is usually synthesized in the laboratory by reverse transcription from messenger RNA extracted from cells.

Contacting: Placing in direct physical association with.

DNA (deoxyribonucleic acid): DNA is a long chain polymer which includes the genetic material of most living organisms (some viruses have genes including ribonucleic acid (RNA)). The repeating units in DNA polymers are four different nucleotides, each of which includes one of the four bases, adenine (A), guanine (G), cytosine (C), and thymine (T) bound to a deoxyribose sugar to which a phosphate group is attached.

Unless otherwise specified, any reference to a DNA molecule is intended to include the reverse complement of that DNA molecule. Except where single-strandedness is required by context, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Expression: The process by which the coded information of a gene is converted into an operational, non-operational, or structural part of a cell, such as the synthesis of a protein.

Fluorescent protein: A protein that has the ability to emit light of a particular wavelength (emission wavelength) when exposed to light of another wavelength (excitation wavelength). Non-limiting examples of fluorescent proteins are the green fluorescent protein (GFP; see, for instance, GenBank Accession Number M62654) from the Pacific Northwest jellyfish, Aequorea victoria and natural and engineered variants thereof (see, for instance, U.S. Pat. Nos. 5,804,387; 6,090,919; 6,096,865; 6,054,321; 5,625,048; 5,874,304; 5,777,079; 5,968,750; 6,020,192; and 6,146,826; and published international patent application WO 99/64592). Other examples include split-GFP and split-GFP variants, folding variants of GFP (e.g., more soluble versions, superfolder versions), spectral variants of GFP which have a different fluorescence spectrum (e.g., YFP, CFP), and GFP-like fluorescent proteins (e.g., DsRed; and DsRed variants, including DsRed1, DsRed2 (see, e.g., Matz et al., Nat. Biotechnol., 17:969-973, 1999).

Fused: Linkage by covalent bonding.

Green fluorescent protein (GFP): As used herein, “GFP” refers to any fluorescent protein that fluoresces green, including fragments, derivatives and variants thereof. GFP includes GFP-like proteins, which may be a fragment, derivative or variant of GFP. For example, derivatives of GFP include enhanced GFP and Emerald. The GFP structure includes eleven anti-parallel outer beta strands and one inner alpha strand. In some embodiments, fragments of GFP are included which do not fluoresce on their own, but will fluoresce when in the presence of the remaining fragment or fragments of GFP, e.g. split-GFP.

Host Cell or Recombinant Host Cell: A cell that has been genetically altered, or is capable of being genetically altered by introduction of an exogenous polynucleotide, such as a recombinant plasmid or vector. Typically, a host cell is a cell in which a vector can be propagated and its DNA expressed. The cell may be prokaryotic or eukaryotic. For example, the host cell may be a bacteria cell, including an E. coli cell. “Host cell” also includes a colony of cells, for example, a colony of E. coli cells. Thus, “contacting a host cell” and “incubating a host cell” include contacting a colony of host cells or incubating a colony of host cells. The term also includes any progeny of the subject host cell. It is understood that all progeny may not be identical to the parental cell since there may be mutations that occur during replication. However, such progeny are included when the term “host cell” is used.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as base pairing. More specifically, A will hydrogen bond to T or U, and G will bond to C. Complementary refers to the base pairing that occurs between two distinct nucleic acid sequences or two distinct regions of the same nucleic acid sequence.

Hydrogel: A colloid gel including an internal phase and a dispersion medium, in which water is the dispersion medium. Non-limiting examples of a hydrogel include mixtures including agar and water or agarose and water. In some embodiments, a hydrogel includes an immobilized affinity reagent; such a hydrogel is also known as a “capture plate.” In some embodiments, a hydrogel contains growth media, for example LB-agar; such a hydrogel is known as a “colony plate.” In some instances, a hydrogel contains both growth media and an immobilized affinity reagent.

Immobilize: To trap or render substantially motionless. For example, an affinity reagent linked to an agarose bead may be immobilized in a hydrogel by adding the affinity reagent to molten components of the hydrogel prior to solidification of the hydrogel; the affinity reagent becomes immobilized in the hydrogel upon solidification of the gel.

Label: A composition detectable by (for instance) spectroscopic, photochemical, biochemical, immunochemical, or chemical means. Typical labels include fluorescent proteins or protein tags, fluorophores, radioactive isotopes (including for instance ³²P), ligands, biotin, digoxigenin, chemiluminescent agents, electron-dense reagents (such as metal sols and colloids), and enzymes (e.g., for use in an ELISA), haptens, and proteins or peptides (such as epitope tags) for which antisera or monoclonal antibodies are available. Methods for labeling and guidance in the choice of labels useful for various purposes are discussed, e.g., in Sambrook et al., in Molecular Cloning: A Laboratory Manual, Cold Spring Harbor Laboratory Press (1989) and Ausubel et al., in Current Protocols in Molecular Biology, John Wiley & Sons, New York (1998). A label often provides or generates a measurable signal, such as radioactivity, fluorescent light or enzyme activity, which can be used to detect and/or quantitate the amount of labeled molecule.

Membrane permeable to soluble protein: A hydrophilic layer of material which serves as a selective barrier to insoluble protein. Soluble protein may pass through the membrane; however, the membrane is substantially impermeable to insoluble protein. The skilled artisan is familiar with membranes that are permeable to soluble protein. For example, Durapore® membrane filter (Millipore Co., Billerica, Mass.; type HVLP; 0.45 μm) is a membrane permeable to soluble protein. In some embodiments, a membrane permeable to soluble protein is also optically transparent. For example, polyethylene, track etched (PETE) membrane.

Multiple cloning site (MCS): A MCS is a region of DNA containing a series of restriction enzyme recognition sequences. Typically, the restriction sites are only present once in the MCS. Vectors and plasmids used for cloning and expression typically contain a MCS to facilitate insertion of a heterologous nucleic acid sequence, such as the coding sequence of a gene of interest. In some embodiments, a MCS including at least two, at least three, at least four, at least five or at least six restriction enzyme recognition sites. The restriction sites may be immediately adjacent, they may overlap, there may be one or more nucleic acids between the sites, or any combination thereof.

Nucleic acid: A polymeric form of nucleotides, which may include both sense and anti-sense strands of RNA, cDNA, genomic DNA, and synthetic forms and mixed polymers thereof. A nucleotide refers to a ribonucleotide, deoxynucleotide or a modified form of either type of nucleotide. The phrase nucleic acid molecule as used herein is synonymous with nucleic acid and polynucleotide. A nucleic acid molecule is usually at least six bases in length, unless otherwise specified. The term includes single- and double-stranded forms. The term includes both linear and circular (plasmid) forms. A polynucleotide may include either or both naturally occurring and modified nucleotides linked together by naturally occurring nucleotide linkages and/or non-naturally occurring chemical bonds and/or linkers.

Nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.). The term nucleic acid molecule also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

Unless specified otherwise, the left hand end of a polynucleotide sequence written in the sense orientation is the 5′-end and the right hand end of the sequence is the 3′-end. In addition, the left hand direction of a polynucleotide sequence written in the sense orientation is referred to as the 5′-direction, while the right hand direction of the polynucleotide sequence is referred to as the 3′-direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Purified: A purified biological component (such as a nucleic acid molecule, protein, or cell) has been substantially separated from other biological components in the cell of the organism, or the organism itself, in which the component naturally occurs, such as other nucleic acid, proteins and cell. The term also embraces nucleic acid molecules and proteins prepared by recombinant expression in a host cell as well as chemically synthesized nucleic acid molecules and proteins. For example, a purified cell is one that is substantially separated from other types of cells or from an organism.

Promoter: A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

Protein: A polymer of amino acid residues, including amino acid polymers in which one or more amino acid residue is an artificial chemical mimetic of a corresponding naturally occurring amino acid, as well as to naturally occurring amino acid polymers and non-naturally occurring amino acid polymer. Multiple polymers of amino acids binding to each other are a protein complex. Protein and polypeptide may be used interchangeably throughout this application and mean at least two covalently attached amino acids, which includes proteins, polypeptides, oligopeptides and peptides.

Sequence identity/similarity: The primary sequence similarity between two nucleic acid molecules, or two amino acid molecules, is expressed in terms of the similarity between the sequences, otherwise referred to as sequence identity. Sequence identity is frequently measured in terms of percentage identity (or similarity or homology); the higher the percentage, the more similar are the two sequences.

Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations.

By way of example, the alignment tools ALIGN (Myers and Miller, CABIOS 4:11-17, 1989) or LFASTA (Pearson and Lipman, 1988) may be used to perform sequence comparisons (Internet Program © 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

Proteins orthologs are typically characterized by possession of greater than 75% sequence identity counted over the full-length alignment with the amino acid sequence of a specific reference protein, using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

When significantly less than the entire sequence is being compared for sequence identity, homologous sequences will typically possess at least 80% sequence identity over short windows of 10-20 amino acids, and may possess sequence identities of at least 85%, at least 90%, at least 95%, or at least 99%. Sequence identity over such short windows can be determined using LFASTA; methods are described at the NCSA Website; also, direct manual comparison of such sequences is a viable if somewhat tedious option.

One of skill in the art will appreciate that the sequence identity ranges provided herein are provided for guidance only; it is entirely possible that strongly significant homologs could be obtained that fall outside of the ranges provided.

The similarity/identity between two nucleic acid sequences can be determined essentially as described above for amino acid sequences. An alternative indication that two nucleic acid molecules are closely related is that the two molecules hybridize to each other (or both hybridize to the same third sequence) under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. The Tₘ of a hybrid molecule can be estimated from the following equation:

Tₘ=81.5 C−16.6(log₁₀[Na⁺])+0.41(% G+C)−0.63(% formamide)−(600/l)

Where l=the length of the hybrid in base pairs.

This equation is valid for concentrations of Na⁺ in the range of 0.01 M to 0.4 M, and it is less accurate for calculations of Tₘ in solutions of higher [Na⁺]. The equation is also primarily valid for DNAs whose G+C content is in the range of 30% to 75%, and it applies to hybrids greater than 100 nucleotides in length.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though wash times also influence stringency. Calculations regarding hybridization conditions required for attaining particular degrees of stringency are discussed by Sambrook et al. (ed.) (Molecular Cloning: A Laboratory Manual, 2nd ed., vol. 1-3, Cold Spring Harbor Laboratory Press, Cold Spring Harbor, N.Y., 1989, chapters 9 and 11), and Tijssen (Laboratory Techniques in Biochemistry and Molecular Biology Part I, Ch. 2, Elsevier, New York, 1993), herein incorporated by reference. The following are exemplary hybridization conditions:

Very High Stringency (Detects Sequences that Share 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share 80% Identity or Greater)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share Greater than 50% Identity)

Hybridization: 6×SSC at RT to 55° C. for 16-20 hours

Wash at least twice: 2×-3×SSC at RT to 55° C. for 20-30 minutes each.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Specifically hybridizable and specifically complementary are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Secretion signal sequence: A protein sequence that can be used to direct a newly synthesized protein of interest through a cellular membrane, including the inner membrane or both inner and outer membranes of prokaryotes as well as organelle and the cell membrane of eukaryotic cells.

Soluble protein: A protein capable of dissolving in a water-based liquid at room temperature and remaining dissolved. The solubility of a protein may change depending on the concentration of the protein in the water-based liquid, the buffering condition of the liquid and the concentration of other solutes in the liquid, for example salt and protein concentration. A soluble protein is one that dissolves to a measurable extend in TNG buffer at room temperature. The soluble proteins described herein may cross a hydrophilic filter having a pore size of 0.45 μm, for example, a Durapore® membrane filter (Millipore Co., Billerica, Mass.; type HVLP; 0.45 μm), when the protein is dissolved in a solution.

Split-Fluorescent Protein (SFP): A protein complex composed of two or more protein fragments that individually are not fluorescent, but, when formed into a complex, result in a functional (that is, fluorescing) fluorescent molecule. Split-GFP is a SFP. Individual protein fragments of a SFP are known as complementing fragments or complementary fragments. Complementing fragments which will spontaneously assemble into a functional fluorescent protein are known as self-complementing, self-assembling, or spontaneously-associating complementing fragments. A complemented fluorescent protein is a protein complex including all the complementing fragments of a SFP necessary for the SFP to be active (i.e., fluorescent). Complemented fluorescent protein fluorescence is the fluorescent signal of a complemented SFP under conditions sufficient to excite the fluorescent protein. Some examples of SFP fragments include SFP tags and SFP detectors.

Split-GFP: A SFP composed of multiple self-assembling protein fragments that individually are not fluorescent, but, when complemented, form a functional fluorescent GFP. See, e.g., U.S. Pat. App. Pub. No. 2005/0221343 and hit. Pat. App. Pub. No. WO/2005/074436, hereby incorporated by reference in their entirety; and Cabantous et al., Nat. Biotechnol., 23:102-107, 2005; Cabantous and Waldo, Nat. Methods, 3:845-854, 2006. The GFP structure includes eleven anti-parallel outer beta strands and one inner alpha strand. Split-GFP includes modified fragments of GFP which do not fluoresce on their own, but will fluoresce when in the presence of the remaining fragment or fragments.

For example, split-GFP may include split-GFP fragments GFP S1-10 and GFP S11. GFP S1-10 corresponds to GFP beta strands 1-10 and GFP S11 corresponds to beta strand 11. Neither molecule fluoresces alone, but will form the complete fluorophore when brought into association. In some embodiments, variations of GFP S1-10, or variations of GFP S11 may be utilized. For example, GFP S1-10 OPT (SEQ ID NO: 4) and GFP S1-10 A4 (SEQ ID NO: 6) may be used as a split-GFP S1-10 fragment. Further, for example, GFP S11 214-238 (SEQ ID NO: 8), GFP S11 214-230 (SEQ ID NO: 10), GFP S11 M1 (SEQ ID NO: 12), GFP S11 M2 (SEQ ID NO: 14), GFP S11 M3 (SEQ ID NO: 16) may be used as a split-GFP S11 fragment. Other variations are also available.

In other examples, split-GFP may include split-GFP fragments GFP S1-9 and GFP S10-11. GFP S1-9 corresponds to GFP beta strands 1-9 and GFP S10-11 corresponds to beta strands 10-11. Neither molecule fluoresces alone, but will form the complete fluorophore when brought into association. In some embodiments, variations of GFP S 1-9, or variations of GFP 510-11 may be utilized. For example, GFP 1-9 OPT (SEQ ID NO: 34) may be used as a split-GFP S1-9 fragment and GFP 10-11 OPT (SEQ ID NO: 36) may be used as a split-GFP S11 fragment.

In other examples, a tripartite system is used that includes GFP S11, GFP S10 and GFP S1-9.

Tag: A polypeptide that, when fused to a heterologous protein or peptide, facilitates the detection or isolation of the heterologous protein. Tags contemplated for use with the compositions and methods described herein include, but are not limited to affinity tags, detection tags and SFP tags. Although tags are often grouped into the aforementioned categories, one of skill in the art will recognize that some tags can be members of more than one group. For example, affinity tags can often be used as a detection tag, and detection tags can often be used as affinity tags. Nucleic acid encoding tags and nucleic acid constructs including nucleic acid sequences encoding tags are known to the skilled artisan and are available commercially.

An affinity tag is a polypeptide that specifically binds to (or with) an affinity reagent. For example, some affinity tags are recognized by an antibody, such as T7, FLAG, hemagglutinin (HA) VSV-G, V5 or c-myc tags. In these cases the antibody is the affinity reagent. Antibodies to these and other affinity tags are commercially available for a variety of sources. Other examples of affinity tags include affinity tags recognized by a recognized by a substrate or compound, such as a histidine tag (e.g., 6HIS; 5HIS), MBP, CBP or GST tags. In this case, the substrate or compound is the affinity reagent. Substrates to these and other affinity tags are commercially available for a variety of sources. For example, histidine tags have affinity for nickel, thus nickel is an affinity reagent for a histidine tag. In some embodiments, the nucleic acid molecules disclosed herein encode a SFP tag, such as GFP S11, GFP, S10, GFP, S1-10, or GFP S1-9. In these cases, an affinity reagent could be the corresponding SFP detector, such as GFP S 1-10 or GFP S1-9.

Tagging is the process of recombinantly (or chemically) attaching a tag to a protein of interest, such as to facilitate detection or isolation of the protein.

Vector: A nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements known in the art. An integrating vector is capable of integrating itself into a host nucleic acid. An expression vector is a vector that contains the necessary regulatory sequences to allow transcription and translation of inserted gene or genes.

### II. Overview of Several Embodiments

Described herein are methods for identifying a protein as soluble in vitro, or both in vitro and in vivo, as well as identifying a soluble protein complex in vitro, or both in vitro and in vivo. Kits and systems for performing the methods are also provided.

A method of identifying a soluble protein is provided. In some embodiments, the method comprises expressing within at least one host cell a first heterologous amino acid molecule comprising a first test protein; bringing the at least one host cell into aqueous contact with the surface of a hydrogel comprising an immobilized affinity reagent with affinity for the first heterologous amino acid molecule for a period of time sufficient for transfer of the first amino acid molecule into the hydrogel; and detecting a complex of the first heterologous amino acid molecule and the immobilized affinity reagent in the hydrogel, wherein the presence of a complex of the first amino acid molecule and the immobilized affinity reagent in the hydrogel identifies the first test protein as a soluble protein.

In some embodiments of the method of identifying a soluble protein, the first heterologous amino acid molecule comprises a detection tag that does not bind the immobilized affinity reagent and detecting the complex of the first heterologous amino acid molecule and the immobilized affinity reagent in the hydrogel comprises detecting the presence of the detection tag immobilized in the hydrogel. In some embodiments, the detection tag is a Split Fluorescent Protein (SFP) tag, the hydrogel comprises a SFP detector, and detecting the presence of the detection tag immobilized in the hydrogel comprises detecting immobilized complemented Split Fluorescent Protein fluorescence in the hydrogel. In some such embodiments, the first test protein is fused to an affinity tag that binds to the immobilized affinity reagent and the affinity tag is on the N-terminus of the first test protein and the detection tag is on the C-terminus of the first test protein; or the affinity tag is on the C-terminus of the first test protein and the detection tag is on the N-terminus of the first test protein.

In some embodiments of the method of identifying a soluble protein, the method further comprises expressing within the host cell a second heterologous amino acid molecule comprising a SFP detector, wherein the first heterologous amino acid molecule comprises a SFP tag, and detecting the complex of the first heterologous amino acid molecule and the immobilized affinity reagent in the hydrogel comprises detecting immobilized complemented Split Fluorescent Protein fluorescence in the hydrogel.

Some embodiments of the method of identifying a soluble protein further comprise identifying a soluble protein complex. In some such embodiments, the methods further comprise expressing within the host cell a second heterologous amino acid molecule comprising a second test protein, wherein the second heterologous amino acid molecule does not bind to the immobilized affinity reagent; wherein detecting the complex of the first heterologous amino acid molecule and the immobilized affinity reagent in the hydrogel comprises detecting a complex of the second heterologous amino acid molecule and the immobilized affinity reagent in the hydrogel; and wherein detecting a complex of the second heterologous amino acid molecule and the immobilized affinity reagent in the hydrogel further identifies the first and second test proteins as a soluble protein complex.

In some embodiments of the method of identifying a soluble protein further comprising identifying a soluble protein complex, the second heterologous amino acid molecule comprises a detection tag, and detecting the complex of the second heterologous amino acid molecule and the immobilized affinity reagent in the hydrogel comprises detecting the presence of the detection tag immobilized in the hydrogel. In some embodiments, the detection tag is a SFP tag, the hydrogel comprises a SFP detector, and detecting the presence of the detection tag immobilized in the hydrogel comprises detecting immobilized complemented Split Fluorescent Protein fluorescence in the hydrogel.

Some embodiments of the method of identifying a soluble protein further comprising identifying a soluble protein complex also comprise expressing within the host cell a third heterologous amino acid molecule comprising a SFP detector, wherein the second heterologous amino acid molecule comprises a second test protein fused to a SFP tag, and detecting the complex of the second heterologous amino acid molecule and the immobilized affinity reagent in the hydrogel comprises detecting immobilized complemented Split Fluorescent Protein fluorescence in the hydrogel.

In some embodiments disclosed herein each heterologous amino acid comprises a secretion signal sequence.

Some embodiments disclosed herein comprise lysing at least one host cell. In some embodiments, lysing at least one host cell comprises contacting at least one host cell with a lysis enzyme; contacting at least one host cell with a detergent; subjecting at least one host cell to a freeze thaw cycle; or a combination of two or more thereof.

In some embodiments of the methods of identifying a soluble protein disclosed herein, the methods comprise selecting the host cell that expresses the soluble protein. In some embodiments, selecting the host cell is performed by a robot.

In some embodiments of the methods of identifying a soluble protein disclosed herein the host cell is a bacteria cell. In some embodiments, host cell is an E. coli cell.

In some embodiments, the SFP tag is a Split-Green Fluorescent Protein (GFP) S11 tag and the SFP detector is split-GFP S1-10.

In some embodiments of the methods described herein, the host cell is separated from the hydrogel by a membrane permeable to soluble protein. In some embodiments, the membrane is optically translucent. In some embodiments, the membrane comprises a position locator element. In some embodiments, the position locator element comprises cells expressing a fluorescent molecule.

In some embodiments comprising position locator element, the methods comprise detecting the position locator element; determining a membrane orientation based on the location of the position locator element; and selecting the host cell comprising the nucleic acid encoding the soluble test protein based on the membrane orientation. In some such embodiments, the determining and selecting steps are performed by a robot.

In some embodiments of the methods described herein, the hydrogel comprises agarose. In some embodiments of the methods described herein, the hydrogel is contained in a dish or plate.

In some embodiments of the methods described herein, the first heterologous amino acid molecule further comprises an affinity tag that binds to the immobilized affinity reagent. In some embodiments, the affinity tag comprises a polyhistidine tag and the immobilized affinity reagent comprises chelated nickel or cobalt.

In some embodiments of identifying a soluble protein that comprise expressing a first and second heterologous amino acid molecule, the first heterologous amino acid molecule is encoded by a first nucleic acid molecule that is operably linked to a first promoter and the second heterologous amino acid molecule is encoded by a second nucleic acid molecule that is operably linked to a second promoter. In some embodiments, the first and second promoters are inducible promoters. In some such embodiments, the first heterologous amino acid molecule comprises a SFP tag, the second heterologous amino acid molecule comprises a SFP detector, and the method further comprises detecting in vivo expression of the first and second amino acid molecules, comprising contacting the host cell with a reagent that induces expression from the first promoter for a period of time sufficient to allow expression of the first heterologous amino acid molecule; contacting the host cell with a reagent that induces expression from the second promoter for a period of time sufficient to allow expression of the second heterologous amino acid molecule; and detecting complemented Fluorescent Protein fluorescence within the host cell, thereby detecting in vivo expression of the first and second heterologous amino acid molecules. In some such embodiments, contacting the host cell with a reagent that induces expression from the first promoter and contacting the host cell with a reagent that induces expression from the second promoter is performed simultaneously or sequentially.

Some embodiments of the method of identifying a soluble protein disclosed herein comprise incubating at least two host cells at separately addressable locations on the surface of the hydrogel, wherein each host cell comprises a different first heterologous amino acid molecule comprising a different first test protein. In some such embodiments, the different test proteins are members of a library of test proteins. In some embodiments, the different test proteins are members of a library of variants of the same protein.

In some embodiments of the method of identifying a soluble protein further comprising identifying a soluble protein complex, the method comprises incubating at least two host cells at separately addressable locations on the surface of the hydrogel, wherein each host cell comprises a first heterologous amino acid molecule encoding a first test protein and a second heterologous amino acid molecule encoding a second test protein, and wherein the at least two host cells comprise: a different first heterologous amino acid molecule comprising a different first test protein; a different second heterologous amino acid molecule comprising a different second test protein; or a combination thereof. In some such embodiments, the different first test proteins, the different second test proteins or a combination thereof are members of a library of test proteins. In some such embodiments, the different first test proteins, the different second test proteins or a combination thereof are members of a library of variants of the same protein.

Also provided are kits for performing the methods described herein. In some embodiments, a kit comprises a nucleic acid construct encoding a SFP tag and a multiple cloning site adjacent thereto, such that an encoding sequence inserted into the multiple cloning site results in a nucleic acid molecule that encodes a test protein fused with the SFP tag; a hydrogel comprising an immobilized affinity reagent; and instructions for carrying out the method.

Also provided are systems for detecting a soluble protein. In some embodiments, a system comprises a first nucleic acid construct encoding a SFP tag and a multiple cloning site adjacent thereto, wherein insertion of a nucleic acid molecule encoding a test protein into the multiple cloning site is expressed as a heterologous amino acid nucleic acid molecule that encodes a heterologous amino acid comprising the test protein and the SFP tag; a second nucleic acid construct encoding a SFP detector or purified SFP detector, or both; a host cell comprising the first nucleic acid construct, the second nucleic acid construct, or both; and, a hydrogel comprising an immobilized affinity reagent with affinity for the first heterologous amino acid molecule. In some embodiments of the provided systems, the first nucleic acid construct encoding the SFP tag and the multiple cloning site adjacent thereto further encodes an affinity tag that binds to the immobilized affinity reagent in the hydrogel, such that an encoding sequence inserted into the multiple cloning site results in a nucleic acid molecule that encodes a heterologous amino acid comprising a test protein fused with the SFP tag and the affinity tag.

### III. In Vitro Identification of Soluble Proteins and Soluble Protein Complexes

Embodiments disclosed herein include methods of identifying a protein as soluble in vitro, as well as methods of identifying a complex of at least two proteins as soluble in vitro.

One provided method of identifying a protein as soluble involves expressing a heterologous amino acid molecule within at least one host cell. The heterologous amino acid molecule includes a test protein. In this method, the at least one host cell is brought into aqueous contact with the surface of a hydrogel for a period of time sufficient for diffusion (transfer) of the first heterologous amino acid molecule into the hydrogel. The hydrogel includes an immobilized affinity reagent with affinity for the heterologous amino acid molecule; thus, if the heterologous amino acid molecule diffuses into the hydrogel it will bind the immobilized affinity reagent. If the heterologous amino acid molecule does not diffuse into the hydrogel, then it will not bind to the immobilized affinity reagent. Detection of a complex of the heterologous amino acid molecule and the immobilized affinity reagent in the hydrogel identifies the test protein as a soluble protein. Detection of a complex of the immobilized affinity reagent and the heterologous amino acid can be accomplished according to standard techniques and as described herein. In some embodiments of this method, the heterologous amino acid molecule also includes a detection tag, an affinity tag, or both. The detection tag does not bind to the immobilized affinity reagent, but the affinity tag does bind to the immobilized affinity reagent. In some examples the detection tag is a SFP S11 tag and the affinity tag is a His tag. In some embodiments of this method where the detection tag is a SFP S11 tag, the host cell also expresses a heterologous amino acid including a SFP detector.

Another provided method includes identifying a soluble protein and a soluble protein complex. This method includes expression of both a first and a second heterologous amino acid molecule in at least one host cell. The first heterologous amino acid molecule includes a first test protein and the second heterologous amino acid molecule includes a second test protein. In this method, the at least one host cell is brought into aqueous contact with the surface of a hydrogel for a period of time sufficient for diffusion of the first heterologous amino acid molecule and the second heterologous amino acid into the hydrogel. The hydrogel includes an immobilized affinity reagent with affinity for the first heterologous amino acid molecule, but not the second heterologous amino acid molecule. If the first heterologous amino acid molecule is soluble, then it will diffuse into the hydrogel and bind to the immobilized affinity reagent. If the first and second heterologous amino acid molecules are soluble and bind to each other, then a complex of the first and second heterologous amino acid molecules will bind to the immobilized affinity reagent in the hydrogel. Because the second heterologous amino acid molecule does not bind to the immobilized affinity reagent, detecting a complex of the second heterologous amino acid molecule with the immobilized affinity reagent identifies (1) the first and second heterologous amino acid molecules a soluble proteins and (2) the first and second amino acid molecules as a soluble protein complex. Detection of a complex of the immobilized affinity reagent and the heterologous amino acid can be accomplished according to standard techniques and as described herein. In some embodiments of this method, the first heterologous amino acid molecule includes an affinity tag and the second heterologous amino acid molecule includes a detection tag. The detection tag does not bind to the immobilized affinity reagent, but the affinity tag does bind to the immobilized affinity reagent. In some examples the detection tag is a SFP S11 tag and the affinity tag is a His tag. In some embodiments of this method where the detection tag is a SFP S11 tag, the host cell also expresses a heterologous amino acid including a SFP detector.

Another provided method includes the methods described above, in combination with a method of identifying total in vivo expression of the heterologous amino acid in the host cell (e.g., quantifying soluble and insoluble expression of the heterologous amino acid). In this method, a heterologous amino acid including a test protein and a SFP S11 tag is expressed in the host cell; additionally, a second heterologous amino acid including a SFP detector is expressed in the host cell. The two heterologous amino acids are simultaneously expressed, leading to complementation of the SFP in the host cell. The fluorescence within the host cell is detected according to standard methods (see e.g., U.S. Patent App. Pub. No 2005/0221343). The fluorescence is proportional to the total expression (soluble and insoluble) of the heterologous amino acid including the test protein and the SFP S11 tag. The total protein expression can be compared with the in vitro identification of a soluble protein using the methods described above.

Another provided method of identifying a protein as soluble involves incubating at least one host cell (which cell includes a first and a second nucleic acid) on the surface of a hydrogel (which hydrogel includes an immobilized affinity reagent specific for a protein encoded by the first or second nucleic acid). In this method, the first nucleic acid encodes a first test protein fused to a SFP tag and the second nucleic acid encodes a SFP detector complementary to the SFP tag. Expression of the first and second nucleic acids yields the SFP tag (fused to the first test protein) and detector, which, if soluble, bind together to form a functional (complemented) fluorescent protein. If the first test protein fused to a SFP tag and the SFP detector protein are capable of transiting from the cells into the hydrogel (e.g., if they are secreted from the cells or at least some of the cells are lysed to release them, and the proteins are sufficiently soluble to pass into the hydrogel), then the affinity reagent will bind to one of these proteins (i.e., the affinity reagent will capture the protein for which it is specific) and the affinity reagent-bound protein will form a complex with the complementary SFP fragment. Detection of the immobilized complemented SFP (for instance, using methods described herein) identifies the first test protein as a soluble protein.

Another provided method of identifying a protein as soluble involves incubating at least one host cell (which cell includes a first nucleic acid) on the surface of a hydrogel (which hydrogel includes (1) an immobilized affinity reagent specific for a protein encoded by the first nucleic acid and (2) SFP detector). In examples of this method, the first nucleic acid encodes a first test protein fused to a SFP tag and the SFP detector in the hydrogel is complementary to the SFP tag. Expression of the first nucleic acids yields the SFP tag (fused to the first test protein). If the first test protein fused to the SFP tag is capable of transiting from the cells into the hydrogel (e.g., if it is secreted from the cells or at least some of the cells are lysed to release it, and the protein is sufficiently soluble to pass into the hydrogel), then the affinity reagent will bind to the first test protein fused to the SFP tag (i.e., the affinity reagent will capture the protein for which it is specific) and the affinity reagent-bound protein will form a complex with the complementary SFP detector present in the hydrogel. Detection of the immobilized complemented SFP (for instance, using methods described herein) identifies the first test protein as a soluble protein.

Another provided method of identifying a protein as soluble involves incubating at least one host cell (which cell includes a first nucleic acid) on the surface of a hydrogel (which hydrogel includes an immobilized affinity reagent specific for a protein encoded by the first nucleic acid). An SFP detector is added to the hydrogel before, during, after incubation of the host cell with the hydrogel, or a combination of two of more thereof. In examples of this method, the first nucleic acid encodes a first test protein fused to a SFP tag and the SFP detector added to the hydrogel is complementary to the SFP tag. Expression of the first nucleic acids yields the SFP tag (fused to the first test protein). If the first test protein fused to the SFP tag is capable of transiting from the cells into the hydrogel (e.g., if it is secreted from the cells or at least some of the cells are lysed to release it, and the protein is sufficiently soluble to pass into the hydrogel), then the affinity reagent will bind to the first test protein fused to the SFP tag (i.e., the affinity reagent will capture the protein for which it is specific) and the affinity reagent-bound protein will form a complex with the complementary SFP detector added to the hydrogel. Detection of the immobilized complemented SFP (for instance, using methods described herein) identifies the first test protein as a soluble protein.

Also provided herein are methods of identifying a soluble protein complex. In one embodiment, the method of identifying a soluble protein complex involves incubating at least one host cell (which cell includes a first, a second and a third nucleic acid) on the surface of a hydrogel (which hydrogel includes an immobilized affinity reagent specific for the protein encoded by the second nucleic acid). In this method, the first nucleic acid encodes a first test protein fused to a SFP tag, the second nucleic acid encodes a second test protein that binds the immobilized affinity reagent, and the third nucleic acid encodes an SFP detector complementary to the SFP tag. Expression of the first and third nucleic acids yields the SFP tag and detector, which, if soluble, bind together to form a functional (complemented) fluorescent protein. Expression of the second nucleic acid yields the second test protein, which if capable of transiting from the cells into the hydrogel (e.g., if it is secreted from the cells, or if at least some of the cells are lysed to release it, and if the protein is sufficiently soluble to pass into the hydrogel), binds the immobilized affinity reagent in the hydrogel. If all three proteins are capable of transiting into the hydrogel, and the first test protein fused to a SFP tag binds to the second test protein (which binds the immobilized affinity reagent), then the three proteins will form tripartite protein complex that is immobilized in the hydrogel and includes a functional SFP. Detection of the immobilized complemented SFP (for instance, using methods described herein) identifies the first and second test proteins as forming or part of a soluble protein complex.

Another provided method of identifying a soluble protein complex involves incubating at least one host cell (which cell includes a first and a second nucleic acid) on the surface of a hydrogel (which hydrogel includes (1) an immobilized affinity reagent specific for a protein encoded by the second nucleic acid and (2) SFP detector). In examples of this method, the first nucleic acid encodes a first test protein fused to a SFP tag and the second nucleic acid encodes a second test protein that binds the immobilized affinity reagent and the SFP detector in the hydrogel is complementary to the SFP tag. Expression of the first nucleic acids yields the SFP tag (fused to the first test protein), which if capable of transiting from the cells into the hydrogel (e.g., if it is secreted from the cells or at least some of the cells are lysed to release it, and the protein is sufficiently soluble to pass into the hydrogel), binds the SFP detector in the hydrogel. Expression of the second nucleic acid yields the second test protein, which if capable of transiting from the cells into the hydrogel (e.g., if it is secreted from the cells or at least some of the cells are lysed to release it, and this protein is sufficiently soluble to pass into the hydrogel), binds the immobilized affinity reagent in the hydrogel. Thus, if the first and second test proteins area capable of transiting from the cells into hydrogel, then a tripartite protein complex that is immobilized in the hydrogel and includes a functional SFP will form. Detection of the immobilized complemented SFP (for instance, using methods described herein) identifies the first and second test proteins as forming or part of a soluble protein complex.

Another provided method of identifying a soluble protein complex involves incubating at least one host cell (which cell includes a first and a second nucleic acid) on the surface of a hydrogel (which hydrogel includes an immobilized affinity reagent specific for a protein encoded by the second nucleic acid). An SFP detector is added to the hydrogel before, during, after incubation of the host cell with the hydrogel, or a combination of two of more thereof. In examples of this method, the first nucleic acid encodes a first test protein fused to a SFP tag and the second nucleic acid encodes a second test protein that binds the immobilized affinity reagent and the SFP detector added to the hydrogel is complementary to the SFP tag. Expression of the first nucleic acid yields the SFP tag (fused to the first test protein), which if capable of transiting from the cells into the hydrogel (e.g., if it is secreted from the cells or at least some of the cells are lysed to release it, and the protein is sufficiently soluble to pass into the hydrogel), binds to the SFP detector added to the hydrogel. Expression of the second nucleic acid yields the second test protein, which if capable of transiting from the cells into the hydrogel (e.g., if it is secreted from the cells or at least some of the cells are lysed to release it, and this protein is sufficiently soluble to pass into the hydrogel), binds the immobilized affinity reagent in the hydrogel. Thus, if the first and second test proteins area capable of transiting from the cells into hydrogel, and the SFP detector is added to the hydrogel, then a tripartite protein complex that is immobilized in the hydrogel and includes a functional SFP will form. Detection of the immobilized complemented SFP (for instance, using methods described herein) identifies the first and second test proteins as forming or part of a soluble protein complex.

In some embodiments of the above methods, adding the SFP detector to the hydrogel includes including a nucleic acid encoding the SFP detector in the host cell that is incubated on the hydrogel; expression of the nucleic acid encoding the SFP detector yields the SFP detector, which, if capable of transiting from the cells into the hydrogel (e.g., if it is secreted from the cells or at least some of the cells are lysed to release it, and the SFP detector is sufficiently soluble to pass into the hydrogel), includes adding a SFP detector to the hydrogel.

The disclosed methods may be applied in virtually any host cell type, including without limitation bacterial cells and mammalian cells. For example E. coli host cells may be used (such as BL21 (DE3) cells). Secretion competent yeast and bacterial cells may be used. The skilled artisan is familiar with such cells. Nucleic acid encoding test proteins, affinity tags, SFP tags, and fusion proteins are typically included in an expression vector introduced into the host cells. Host cells, vectors, nucleic acids and methods of use are known to the skilled artisan and further described herein.

The host cell may be contained within a colony of host cells, for example, an E. coli host cell may be contained within a bacterial colony, which bacterial colony is incubated on the surface of a hydrogel. The bacterial colony is preferably about 0 5 mm in diameter, though any size may be used. For example, the bacterial colony may be about 0.1, 0.2, 0.3, 0.4, 0.5, 0.6, 0.7, 0.8, 0.9, 1.0, 1.5, 2.0 or about 2 4 mm in diameter, or even larger or smaller. In some embodiments the host cells are separated from the hydrogel by a membrane permeable to soluble protein. The membrane is useful for transfer of the host cell, for example transfer of the host cells between growth media, hydrogel containing affinity reagents, media containing inducers, media containing lysis reagents, etc. Such transfer of a membrane separating the host cell from the hydrogel is further described herein. The membranes used herein are permeable to soluble protein, but substantially impermeable to insoluble protein. Such membranes are known to the skilled artisan and are further described herein. The skilled artisan is familiar with membranes that are permeable to soluble protein. For example, Durapore® membrane filter (Millipore Co., Billerica, Mass.; type HVLP; 0.45 μm) is a membrane permeable to soluble protein. Typically, the membranes for use with the disclosed methods have a pore dimension of about 0.2 to 0.45 μM. In some embodiments, a membrane permeable to soluble protein is also optically transparent. For example, polyethylene, track etched (PETE) membrane. Use of an optically transparent membrane allows detection of fluorescence (e.g., the fluorescent signal of an immobilized SFP) in the hydrogel without having to remove the membrane from the surface of the hydrogel.

In some examples, a nucleic acid sequence encoding a test protein is operably linked to a nucleic acid sequence encoding a SFP tag. The nucleic acid sequence may or may not be further operably linked to a promoter, for example an inducible promoter. In some examples, one or more nucleic acid sequences encoding a test protein are operably linked to one or more promoters, for example one or more inducible promoters, as described herein. For example, the nucleic acid coding sequence of a test protein is operably linked to a nucleic acid sequence encoding a SFP tag, and placed under the control of a first independently inducible promoter. The coding sequence of the corresponding SFP detector is placed under the control of a second independently inducible promoter. The two resulting nucleic acid constructs may be incorporated into the same or different vectors (i.e., one or two plasmids), provided that the promoters remain separately inducible, and host cells are transformed or transfected with the vectors. In some embodiments, cells carrying the constructs(s) are initially cultured under baseline conditions permitting the repression of both of the independently inducible promoters and subsequently cultured under conditions permitting expression from one or both promoters, either simultaneously or sequentially.

The methods described herein are easily extended to method involving libraries of test proteins, for example a library of variants of a particular protein. In this case, the methods are used to screen a library of nucleic acids encoding a corresponding library of test proteins for soluble test protein. The skilled artisan is familiar with nucleic acid libraries, and such libraries, as well as methods of making them are further described herein. The disclosed methods involving use of libraries of test proteins include at least two host cells, each expressing a different member of the library of test proteins, at separately addressable locations on the surface of the hydrogel.

In embodiments described herein, incubating at least one host cell on the surface of a hydrogel includes bringing at least one host cell into aqueous contact with the surface of a hydrogel.

In embodiments described herein, a host cell including a nucleic acid sequence encoding a heterologous amino acid molecule (e.g., a host cell including a nucleic acid sequence encoding a test protein) is a host cell that will express a heterologous amino acid (e.g., the test protein) under conditions sufficient for expression of the heterologous amino acid molecule within the host cell.

Lysis of Host Cells and Secretion from Host Cells

Several embodiments described herein include lysing at least one host cell to release protein from the host cell. This releases protein from the host cell and makes it available for transfer into the hydrogel. Various methods of lysing host cells are available to the skilled artisan; the methods described herein involve non-denaturing lysis methods. In the case where the host cell is part of a bacterial or mammalian cell colony, lysing at least one host cell does not require lysing all the host cells in the bacterial colony; thus, leaving some viable cells for further study.

In some embodiments, lysing at least one host cell involves contacting the cell with a lysis enzyme. Such enzymes are known to the skilled artisan and are available commercially. For example lysozyme is a lysis enzyme for use on bacterial cells and is available from many supplies, including Sigma-Aldrich (Cat. No. L7651, St. Louis, Mo.). In some embodiments, lysing at least one host cell involves contacting the cell with a non-denaturing detergent. Such detergents are known to the skilled artisan and are available commercially, for example SoluLyse® protein extraction reagent (Genlantis, San Diego, Calif.) and BugBuster® Protein extraction reagent (EMD chemicals, Gibbstown, N.J.).

Various means of applying a lysis enzyme or a non-denaturing detergent to a host cell are available to the skilled artisan. For example, application may be performed by misting the host cells using a spray bottle as described herein. Alternatively, in the case where the host cells are incubated on a membrane, application may be performed by resting the membrane on which the host cells are incubating on an absorbent material (such as blotter paper), in which the lysis enzyme or non-denaturing detergent has been absorbed, for a period of time sufficient to allow partial lysis of the host cells.

The lysis enzyme or non-denaturing detergent may be added to solutions containing various buffers, salts and other chemicals for use in the lysis methods described herein. For example, Tris-based buffers may be used, including TNG buffer. The skilled artisan will understand methods of making such buffers, further many such buffers are disclosed herein.

In some embodiments where the host cells are incubated on a membrane, lysing at least one host cell involves exposing the cells to a freeze thaw cycle. For example, the membrane on which the host cells are incubating may be placed in a freezer for a period of time sufficient to freeze the host cells on the membrane, after which the membrane is removed from the freezer and the host cells are allowed to thaw. Host cells may be exposed to multiple freeze thaw cycles. Lysing at least one host cell may involve a combination of two or more of contacting the host cell with a lysis enzyme, contacting the host cell with a non-denaturing detergent or exposing the host cell to a freeze thaw cycle as described herein.

In some embodiments, the heterologous amino acid molecules (e.g., test protein/SFP tag fusion protein and/or an SFP detector) are secreted from the host cell. Secretion of protein from the host cell allows transit in to the hydrogel (if the protein is sufficiently soluble). Secretion of heterologous amino acid molecules (e.g., test protein) from the host cell may be facilitated by a secretion signal sequence included within the heterologous amino acid molecule (e.g., fused to a test proteins). The secretion signal sequence may be endogenous to the host cells, or may be exogenous. Secretion signal sequences are known to the skilled artisan. See, for example, U.S. Pat. Nos. 4,963,495; 5,840,523; 6,875,590 and Sambrook et al. Molecular Cloning 17.31 (1989), which provides examples of expression vectors designed for the secretion of heterologous proteins in E. coli.

Following application of a lysis reagent, or inducement of secretion of protein from the host cells, the host cells are incubated on the surface of the hydrogel (or on the membrane on the surface of the hydrogel) for a period of time sufficient to allow transfer of soluble protein from the host cell to the hydrogel. For example, the host cells may be incubated for about 5, 10, 15, 30, 45 or 60 minutes, or even more or less time. In some embodiments, the host cells are incubated for about 1, 2, 3, 4, 5, 6, 7, 8, 9, 10, 11, 12, 16, 20 or 24 hours. The skilled artisan will understand the time required for incubation.

### Hydrogels Including Affinity Reagents and SFP Detectors

The disclosed methods involve hydrogels including affinity reagents. Hydrogels and affinity reagents are known to the skilled artisan and are further described herein.

The hydrogels disclosed herein include immobilized affinity reagents, which are affinity reagents (e.g., a substrate capable of binding to an affinity tag) that are rendered substantially motionless by incorporation within a solidified hydrogel. Substrates include metal ions (e.g., copper, nickel, cobalt ions, among others), proteins (e.g., antibodies) and peptides (e.g., glutathione). For example, metal bound resins used in immobilized metal ion affinity chromatography (IMAC) are affinity reagents. Typically, the substrate is linked to a solid support, e.g., an agarose or other composition bead or surface. Solidification of the hydrogel around the solid support traps solid support in the hydrogel, rendering the affinity reagent linked to the solid support substantially motionless in relation to the hydrogel. IMAC, solid supports and agarose beads are well known to the skilled artisan (see, e.g., Block et al., Methods Enzymol., 463:439-473, 2009; Ausubel et al., Current Protocols in Molecular Biology, Greene Publishing Associates, 1992 (and Supplements to 2000)). Non-limiting examples of affinity reagents linked to an agarose bead include Talon® metal affinity resin, Ni-NTA agarose (available from many suppliers, e.g., Qiagen, Valencia, Calif.) and His60 Ni Superflow™ metal affinity resin (Clontech, Mountain View, Calif.). Other agarose bead-based affinity reagents for protein capture are well known to the skilled artisan (Gräslund et al., Nat. Methods 5(2):135-146, 2008). For example amylose could be used to capture maltose binding protein tags (Nallamsetty and Waugh, Protein Expr. Purif., 45:175-182, 2006; Nallamsetty and Waugh, Nat. Protoc., 2:383-391, 2007), or glutathione conjugated beads to capture glutathione-S-transferase tags Goda et al., Protein Sci., 13:652-658, 2004).

The affinity reagent for use in the methods described herein is one that is specific for a heterologous amino acid molecule encoded by a nucleic acid included with a host cell of the disclosed methods. For example, if a heterologous amino acid molecule encoded by a nucleic acid included with a host cell of the disclosed methods includes a 6HIS tag, then the affinity reagent within the hydrogel will be an affinity reagent that binds to a 6HIS tag, such as Talon® metal affinity resin.

Hydrogels include various gelling agents including agar, agarose, agaropectin, a combination of two or more thereof, or other gelling agents. Other gels include silica aerogels, polyacrylamide, calcium-crosslinked alginate, for example. The only requirement is that the pore size of the gel allows analyte molecules to diffuse (that is, the pore size of the gel is not smaller than the analyte hydrodynamic radius). A wide variety of agar is available to the skilled artisan from multiple sources, including Sigma Cat. No. A5306 (Sigma-Aldrich Co., St. Louis, Mo.). A wide variety of agarose is available to the skilled artisan from multiple sources, including Sigma Cat. Nos. A4018, A6236, A6361, A6111, A5986, A5986, A9045, A9414, A6560, A0701, A4679, A2929, A3054, A9539, A4718, A9311, A6013, A0169, A0576, A6877, A9918, A6138, A9793, A3643, A5030, A2576, A3768, A3893, A7174, A4905, A3038, A3038, A7299, A2790, A7431, A5304, 05073, A5093 and U0507 (Sigma-Aldrich Co., St. Louis, Mo.). An agarose-based hydrogel will typically include 0.1 to 1.5% agarose by weight, though higher or lower concentrations of agarose are also contemplated for use in the methods described herein. Methods of constructing agar and agarose based hydrogels are well known to the skilled artisan (see, e.g., Ausubel et al., Current Protocols in Molecular Biology, Greene Publishing Associates, 1992 (and Supplements to October 2010). For example, the components of the hydrogel (e.g., agarose, water and an affinity reagent linked to an agarose bead) may be heated until the components form a molten mixture, and then allowed to cool.

Various buffers, salts, chemicals, etc., may be used in the construction of the hydrogel. For example, the hydrogel may be prepared with a Tris-based buffer such as 150 mM Tris-HCl pH 7.5 or TNG buffer. In some embodiments, 150 mM Tris-HCl, pH 7.5 buffer is used to prepare hydrogels for methods involving detection of a soluble protein, and TNG buffer is used to prepare hydrogels for detection of soluble protein complexes. The skilled artisan will understand how to incorporate buffer into a hydrogel, for example the gelling agent may be mixed with a particular buffer prior to heating, or a solidified hydrogel may be immersed in a particular buffer.

Additionally, hydrogels for use with the disclosed methods may include nutrient reagents. For example, the hydrogel may be Luria-Bertani (LB) agar. In embodiments where the host cell is incubated directly on the surface of a hydrogel that contains the immobilized affinity reagent, the hydrogel typically contains growth media, for example the hydrogel may be a LB-agar based hydrogel that contains the affinity reagent. In these examples, soluble protein released from the host cell may transit directly into the hydrogel for binding to the affinity reagent.

Hydrogels described herein act as a selective barrier to insoluble protein. For example, sieving typically begins in 1% agarose hydrogel for hydrodynamic radii greater than 5,000,000 [Please provide units], which is approx. 0.1 nm (see, e.g., Gosnell and Zimm, Macromolecules, 26 (6), 1304-1308, 1993; Pluen et al., Biophys J., 77: 542-552, 1999).

In embodiments where the host cell is separated from the hydrogel by a membrane, the hydrogel may or may not contain growth media. For example, in some embodiments, the host cell is initially incubated on a membrane that is resting on a growth media plate, for example an LB-agar plate and the membrane is subsequently transferred to a hydrogel including an immobilized affinity reagent, which hydrogel may or may not contain growth media.

Preparation of a hydrogel including an affinity reagent may be done by mixing the affinity reagent with an agarose/water mixture when the mixture is molten (that is, prior to solidification of the hydrogel); the affinity reagent becomes immobilized in the hydrogel upon solidification of the hydrogel. Methods of preparing a hydrogel including an immobilized affinity reagent are further described herein.

In some embodiments, the SFP detector is added to the hydrogel directly, for example, in embodiments where the host cell does not include a nucleic acid encoding a SFP detector. For example, the SFP detector may be added to the hydrogel by overlaying the hydrogel with the SFP detector and allowing it to diffuse into the hydrogel. The SFP detector may be added to the hydrogel before, during and/or after incubation of the host cell with the hydrogel. Purified SFP detector for addition to the hydrogel can be prepared according to standard methods (see, e.g., Ausubel et al., Current Protocols in Molecular Biology, Greene Publishing Associates, 1992 (and Supplements to October 2010), and is also described in U.S. Pat. App. Pub. No. 2005/0221343 and PCT Pub. No. WO/2005/074436. The skilled artisan will understand how to add the SFP detector to the hydrogel; such methods are further described herein.

In some examples, the hydrogel is contained within a container or plate, as described herein.

### Tags

A nucleic acid sequence encoding a heterologous amino acid molecule (e.g., a heterologous amino acid molecule including a test protein) may be operably linked to a nucleic acid sequence encoding an affinity tag that has affinity for the immobilized affinity reagent within the hydrogel. For example, the nucleic acid encoding the test protein may be operably linked to nucleic acid encoding a histidine affinity tag (e.g., 6HIS), in which case the hydrogel will contain an immobilized affinity reagent that binds to histidine, such as Talon® metal affinity resin. In some examples where a nucleic acid encoding a test protein is operably linked to both a SFP tag and an affinity tag, the affinity tag is on the N-terminus of the test protein and the SFP tag is on the C-terminus of the test protein. In other examples, the affinity tag is on the C-terminus of the test protein and the SFP tag is on the N-terminus of the test protein.

In other examples, a nucleic acid encoding the SFP detector corresponding to the SFP tag is operably linked to an affinity tag. For example, the nucleic acid encoding the SFP detector corresponding to the SFP tag may be operably linked to nucleic acid encoding a histidine affinity tag (e.g., 6HIS), in which case the hydrogel will contain an immobilized affinity reagent that binds to histidine, such as Talon® metal affinity resin. Vectors encoding affinity tags for use in the disclosed methods are described herein.

In some examples, a heterologous amino acid sequence including a test protein may also include detection tag. In some examples, a nucleic acid sequence encoding a test protein may be operably linked to a nucleic acid sequence encoding a detection tag.

Detection tags include any polypeptide that, when fused to a heterologous protein or peptide, facilitates the detection or isolation of the heterologous protein. Non-limiting examples of detection tags include full-length fluorescent protein such as those described herein, fragments of SFPs such as those described herein, an affinity tag, and chemical modification. The skilled artisan is familiar with the use of detection tags and will recognize the application of particular detection tags in the methods described herein.

### Detection of Soluble Protein Immobilized in the Hydrogel

Following incubation of at least one host cell on the surface of the hydrogel (or on the membrane on the surface of the hydrogel) for a period of time sufficient to allow transfer of soluble protein from the at least one host cell to the hydrogel, detection of immobilized protein in the hydrogel takes place. In embodiments where a membrane is utilized, the membrane may be removed from the hydrogel prior to imaging immobilized fluorescence in the hydrogel. In some examples, at least about 30 minutes, about 1, 2, 3, 4, 5, 6, 7, 8, 9, 10, 12, 16, 20, or 24 hours, or even more or less time is allowed between incubation of the host cells on the hydrogel (or expression of protein froth host cells) and detection of the immobilized protein in the hydrogel. This time period allows diffusion of any mobilized proteins in the hydrogel, which reduces background measurements of protein in the hydrogel. Detection of protein in the hydrogel can be accomplished according to numerous methods that will be apparent to the skilled artisan. For example, detection of protein in the hydrogel can be accomplished using protein dyes or stains (e.g., SYPRO® Orange, Sigma-Aldrich Cat. No. 55692, St. Louis, Mo.), affinity reagents, detection tags, etc.

In some embodiments, following incubation of the host cells on the surface of the hydrogel (or on the membrane on the surface of the hydrogel) for a period of time sufficient to allow transfer of soluble protein from the host cell to the hydrogel, detection of immobilized fluorescent protein in the hydrogel takes place. In embodiments where a membrane is utilized, the membrane may be removed from the hydrogel prior to imaging immobilized fluorescence in the hydrogel. In some examples, at least about 30 minutes, about 1, 2, 3, 4, 5, 6, 7, 8, 9, 10, 12, 16, 20, or 24 hours, or even more or less time is allowed between incubation of the host cells on the hydrogel (or expression of protein froth host cells) and detection of the immobilized fluorescent protein in the hydrogel. This time period allows diffusion of any mobilized fluorescence in the hydrogel, which reduces background measurements of protein in the hydrogel. Detection of punctate spots of fluorescence (or fluorescent speckles) in the hydrogel indicates a immobilize fluorescent signal (that is, the signal given off by a soluble test protein SFP tag fusion immobilized to the affinity reagent in the hydrogel and complemented to the SFP detector), whereas detection of diffuse fluorescence indicates a lack immobilized protein (corresponding to lack of a soluble test protein).

Detection of immobilized fluorescence in a hydrogel (e.g., the fluorescence of a fluorescent protein bound to a immobilize affinity reagent in the hydrogel) as well as detection of fluorescence in host cells is accomplished according to standard methods of detecting fluorescent proteins. The hydrogel (or host cells) are exposed to the excitation wavelength of the fluorescent protein of interest, and light emitted at the corresponding emission wavelength is detected. Such methods are well known the skilled artisan, and systems for detecting fluorescent proteins are commercially available. For example, the Illumatool system has been previously described for detection of split fluorescent proteins in host cells, including gin bacterial colonies (see, e.g., Cabantous et al., Nat. Biotechnol., 23:102-107, 2005).

### Expression Vectors

Nucleic acid encoding one or more test proteins can be included in one or more expression vectors to direct expression of test protein nucleic acid sequence. Thus, other expression control sequences including appropriate promoters, enhancers, transcription terminators, a start codon at the front of a protein-encoding sequence, splicing signal for introns, maintenance of the correct reading frame of that gene to permit proper translation of mRNA, and stop codons can be included in an expression vector. Generally expression control sequences include a promoter, a minimal sequence sufficient to direct transcription.

Nucleic acid sequences encoding SFP tags, affinity tags and SFP detectors, etc., may be included in an expression vector to direct expression of the corresponding nucleic acid sequence. Optionally, the nucleic acid sequences encoding an SFP tag, affinity tag and/or SFP detector may be operably linked to the nucleic acid encoding a test protein, such that expression from the expression vector results in a fusion protein of the test protein fused to the SFP tag, affinity tag and/or SFP detector.

As will be appreciated by the skilled artisan, expression vectors used to express test proteins, SFP tags, affinity tags, SFP detectors and fusions thereof must be compatible with the host cell in which the proteins are to be expressed. Similarly, various promoter systems are available and should be selected for compatibility with cell type, strain, etc. Codon optimization techniques may be employed to adapt sequences for use in other cells, as is well known.

The expression vector typically contains an origin of replication, a promoter, as well as specific genes which allow phenotypic selection of the transformed cells (e.g., an antibiotic resistance cassette). Vectors suitable for use include, but are not limited, to the pMSXND expression vector for expression in mammalian cells (Lee and Nathans, J. Biol. Chem. 263:3521, 1988). Generally, the expression vector will include a promoter. The promoter can be inducible or constitutive. In one embodiment, the promoter is a heterologous promoter.

Unlike constitutive promoters, an inducible promoter is not always active. Some inducible promoters are activated by physical stimuli, such as the heat shock promoter. Others are activated by chemical stimuli, such as IPTG or Tetracycline (Tet), or galactose. Inducible promoters or gene-switches are used to both spatially and temporally regulate gene expression. Thus, for a typical inducible promoter in the absence of the inducer, there would be little or no gene expression while, in the presence of the inducer, expression should be high (i.e., off/on). The skilled artisan is familiar with inducible promoters and will appreciate which inducible promoters may be used in the embodiments described herein.

In some embodiments, multiple inducible promoters are included on an expression vector, each promoter induced by a different inducer. In other embodiments, multiple expression vectors are included in the host cell, each expression vector including an inducible promoter, each inducible promoter induced by a different inducer. In this way, expression of multiple proteins in a host cell can be independently under the control of separate inducible promoters. Thus, in some embodiments, host cells are engineered to express one or more complementary fragments of a SFP, one or more of which are fused to one or more test proteins. The fragments may be expressed simultaneously or sequentially.

Systems of two independently controllable promoters have been described and are well known in the art, and are described herein. See, for example, Lutz and Bujard, Nucleic Acids Res., 25:1203-1210, 1997.

In one example, a vector in which the promoter is under the repression of the Laclq protein and the arabinose inducer/repressor may be used for expression of the SFP detector (e.g., pPROLAR vector available from Clontech, Palo Alto, Calif.). Repression is relieved by supplying IPTG and arabinose to the growth media, resulting in the expression of the SFP detector. In this system, the araC repressor is supplied by the genetic background of the host E. coli cell. For the controlled expression of the test protein-SFP tag fusion, a vector in which the test protein-SFP tag fusion is under the repression of the tetracycline repressor protein may be used (e.g., pPROTET vector; Clontech). In this system, repression is relieved by supplying anhydrotetracycline to the growth media, resulting in the expression of the test protein-SFP tag fusion construct. The tetR and Laclq repressor proteins may be supplied on a third vector, or may be incorporated into the fragment-carrying vectors.

In using the above system for sequential expression of the test protein-SFP tag fusion, followed by the SFP detector, the addition of anhydrotetracycline to cells transformed with the above constructs displaces the Tet repressor, and expression of the test protein-SFP tag fusion is induced. If the cells are on a membrane, they are then transferred to new plates with fresh media, and the anhydrotetracycline is allowed to diffuse into the media for approximately 1 hour, after which the Tet repressor again binds to the promoter, shutting off expression. The separately inducible T7 promoter is then activated by the addition of IPTG, inducing the expression of the SFP detector. Expression of the SFP detector proceeds for a time sufficient to permit self-complementation with a soluble first fragment-test protein fusion. The membrane can then be moved the hydrogel including the immobilized affinity reagent for capture of protein, or the membrane can be imaged using the optional in vivo soluble protein and soluble protein complex identification methods described herein. This system can easily be adapted for the simultaneous induced expression of test protein-SFP tag fusion, followed by the SFP detector, as will be appreciated by the skilled artisan.

In one example, nucleic acid encoding a test protein is located downstream of the desired promoter. Optionally, an enhancer element is also included, and can generally be located anywhere on the vector and still have an enhancing effect. However, the amount of increased activity will generally diminish with distance.

Expression vectors including a nucleic acid encoding a test protein can be used to transform host cells. Hosts can include isolated microbial, yeast, insect and mammalian cells, as well as cells located in the organism. For example, the host cell may be an E. coli cell, such as an E. coli BL21 (DE3) strain cell.

A “transfected cell” is a cell into which (or into an ancestor of which) has been introduced, by means of recombinant DNA techniques, a DNA molecule encoding a protein of interest. Transfection of a host cell with recombinant DNA may be carried out by conventional techniques as are well known in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such as a CHO cell, such methods of transfection of DNA as calcium phosphate coprecipitates, conventional mechanical procedures such as microinjection, electroporation, insertion of a plasmid encased in a liposome, or virus vectors may be used. Eukaryotic cells can also be cotransformed with DNA sequences encoding the test protein, and a second foreign DNA molecule encoding a selectable phenotype, such as neomycin resistance. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). Other specific, non-limiting examples of viral vectors include adenoviral vectors, lentiviral vectors, retroviral vectors, and pseudorabies vectors.

In some embodiments, test proteins may be cloned into the N-6His pTET S11 SpecR ColE1 ORI vector and transformed into BL21(DE3) bearing the pET GFP 1-10 KanR p15 ORI vector as previously described (Cabantous et al., Nat. Biotechnol., 23:102-107, 2005). In some examples, GFP 1-10 in the pET GFP 1-10 KanR p15 ORI vector may be replaced with dsRed (Clontech, Mountain View, Calif.).

### Selecting a Host Cell Including a Nucleic Acid Encoding a Soluble Protein

Some embodiments further include selecting a host cell including nucleic acid encoding a protein or proteins identified as soluble using the methods described herein. For example, some embodiments include selecting the host cell including a nucleic acid encoding a protein identified as soluble, so that the nucleic acid may be isolated from the host cell. Other examples include selecting the host cell including a first and second nucleic acid encoding first and second proteins identified as forming a soluble protein complex of at least two proteins, so that the nucleic acids may be isolated from the host cell. As used herein, selecting a host cell includes selecting a particular host cell, as well as selecting a number of cells (e.g., a colony of host cells) including the host cell.

Selecting a host cell including nucleic acid encoding a protein or proteins identified as soluble involves identifying the host cell corresponding to the detected soluble protein, and selecting the identified host cell.

In some embodiments, selecting a host cell including nucleic acid encoding a protein or proteins identified as soluble involves identifying the host cell corresponding to the detected immobilized SFP fluorescence used to identify a protein as soluble or to identify a soluble protein complex, and selecting the identified host cell. Methods of identifying the host cell that corresponds to detected immobilized SFP fluorescence are known to the skilled artisan and are further described herein. For example, when the host cell is incubated on the surface of a hydrogel as described herein, the immobilized SFP fluorescence will be detected in the area of the hydrogel beneath the host cell. Thus, identification of the host cell involves identifying a host cell located above the immobilized SFP fluorescence detected in the hydrogel.

In some embodiments, the host cell is separated from the hydrogel by a membrane. In some embodiments where the membrane is an optically transparent membrane, selecting the host cell simply involves identifying the host cell corresponding to detected soluble protein and selecting the host cell.

Methods of selecting a host cell are well known to the skilled artisan and are described herein. In some embodiments, selecting the host cell includes manual selection of the host cell, for example, by picking a colony including the host cell using a sterile toothpick. In some embodiments, selecting the host cell includes robotic selection of the host cell, for example by a colony picking robot. Such robots and methods of using such robots are known to the skilled artisan; also such robots are available commercially, for example from Norgren Systems (No. CP 700; Ronceverte, W.V.) and BioRad (VersArray, No. 2856; Hercules, Calif.). Use of additional robotic technologies, including DNA and protein purification robots and liquid handling robots is included. Such robots are known to the skilled artisan. In some embodiments the selected host cell is cultured for further study.

In some embodiments, a guided host cell identification method is used. Such methods are known to the skilled artisan and are further described herein. In such embodiments, a position locator element that can be differentially detected from the host cell is used. The position locator element may be any means that allows orientation of the at least one host cell in relation to the position locator element. For example, in embodiments where the at least one host cell expresses a fluorescent molecule or a split fluorescent molecule, the position locator element may be fluorescent molecule that can be detected at a different wavelength of light than the fluorescent molecule within the host cells. In some embodiments, the position locator element

In some embodiments, the position locator includes an affinity tag that binds to the immobilized affinity reagent in the hydrogel. In some embodiments, the position locator element is fused with a secretion signal sequence. The position locator element may be expressed by a cell or cells that are incubated on the surface of the hydrogel along with the at least one host cell including the nucleic acid(s) encoding test proteins and SFP fragments. The immobilized complemented SFP and immobilized position locator element are differentially detected and imaged in the hydrogel. Additionally, cells expressing the position locator element may be detected and imaged. The two images are aligned, thereby allowing orientation of the membrane and identification of a host cell on the surface of the membrane that corresponds to detected immobilized SFP fluorescence detected in the hydrogel.

In some embodiments the SFP tag is GFP S11, the SFP detector is GFP S1-10, the immobilized affinity reagent is chelated cobalt linked to an agarose bead (e.g., Talon® metal affinity resin and the position locator element is soluble 6-His tagged dsRed protein as described herein).

In some embodiments where a membrane separates the at least one host cell from the hydrogel, the position locator element includes markings (e.g., a grid pattern) on the membrane that allows orientation of the host cells in relation to the position locator element.

### IV. Fluorescent and Split Fluorescent Proteins

The embodiments described herein involve fluorescent proteins. Non-limiting examples of fluorescent proteins are the green fluorescent protein (GFP; see, for instance, GenBank Accession Number M62654) from the Pacific Northwest jellyfish, Aequorea victoria and natural and engineered variants thereof, including structural variants of GFP, monomeric versions, folding variants of GFP (e.g., more soluble versions, superfolder versions), spectral variants of GFP which have a different fluorescence spectrum (e.g., YFP, CFP), and GFP-like fluorescent proteins (e.g., DsRed). (See, e.g., U.S. Patent Nos. 5,804,387; 6,090,919; 6,096,865; 6,054,321; 5,625,048; 5,874,304; 5,777,079; 5,968,750; 6,020,192; and 6,146,826; and published international patent application WO 99/64592).

GFP and its numerous related fluorescent proteins are now in widespread use as protein tagging agents (for review, see Verkhusha et al., 2003, GFP-like fluorescent proteins and chromoproteins of the class Anthozoa. In: Protein Structures: Kaleidoscope of Structural Properties and Functions, Ch. 18, pp. 405-439, Research Signpost, Kerala, India). GFP-like proteins are an expanding family of homologous, 25-30 kDa polypeptides sharing a conserved 11 beta-strand “barrel” structure. The GFP-like protein family currently includes some 100 members, cloned from various Anthozoa and Hydrozoa species, and includes red, yellow and green fluorescent proteins and a variety of non-fluorescent chromoproteins. A wide variety of fluorescent protein labeling assays and kits are commercially available, encompassing a broad spectrum of GFP spectral variants and GFP-like fluorescent proteins, including cyan fluorescent protein, blue fluorescent protein, yellow fluorescent protein, etc., DsRed and other red fluorescent proteins (Zimmer, Chem. Rev., 102:759-781, 2002; Zhang et al., Nature Rev., 3:906-918, 2002; Clontech, Palo Alto, Calif.; Amersham, Piscataway, N.J.). Typically, GFP variants share about 80%, or greater sequence identity with SEQ ID NO: 2 (or SEQ ID NO: 8). Color-shift GFP mutants have emission colors blue to yellow-green, increased brightness, and photostability (Tsien, Ann. Rev. Biochem., 67:509-544, 1998).

Additional GFP-based variants having modified excitation and emission spectra (see, e.g., U.S. Patent App. Pub. No. 2002/0123113), enhanced fluorescence intensity and thermal tolerance (see, e.g., U.S. Pat. App. Pub. No. 2002/0107362; U.S. Pat. App. Pub. No. 20020177189), and chromophore formation under reduced oxygen levels (see, e.g., U.S. Pat. No. 6,414,119) have also been described. GFPs from the Anthozoans Renilla reniformis and Renilla kollikeri have also been described (see e.g., U.S. Pat. App. Pub No. 2003/0013849 and U.S. Pat. No. 7,528,242).

One widely utilized red fluorescent protein was isolated from Discosoma species of coral, DsRed (Matz et al., Nat. Biotechnol., 17:969-973, 1999), and various DsRed variants (e.g., DsRed1, DsRed2) have been described. DsRed and the other Anthozoa fluorescent proteins share only about 26-30% amino acid sequence identity to the wild-type GFP from Aequorea victoria, yet all the crucial motifs are conserved, indicating the formation of the 11-stranded beta-barrel structure characteristic of GFP. The crystal structure of DsRed has also been solved, and shows conservation of the 11-stranded beta-barrel structure of GFP MMDB Id: 5742.

A number of mutants of the longer wavelength red fluorescent protein DsRed have also been described. In some embodiments, recently described DsRed mutants with emission spectra shifted further to the red may be employed (Wiehler et al., FEBS Letters, 487:384-389, 2001; Terskikh et al., Science, 290:1585-1588, 2000; Baird et al., Proc. Natl. Acad. Sci. U.S.A., 97:11984-11989, 2000). Recently, a monomeric variant of DsRed was described (Campell et al., Proc. Natl. Acad. Sci. USA 99: 7877-7882, 2002). This variant, termed “mRFP1,” matures quickly (in comparison to wild type DsRed, which matures over a period of 30 hours), has no residual green fluorescence, and has excitation and emission wavelengths of about 25 nm longer than other DsRed variants.

Additionally, fluorescent proteins from Anemonia majano, Zoanthus sp., Discosoma striata, Discosoma sp. and Clavularia sp. have also been reported (Matz et al., Nat. Biotechnol., 17:969-973, 1999). A fluorescent protein cloned from the stony coral species, Trachyphyllia geoffroyi, has been reported to emit green, yellow, and red light, and to convert from green light to red light emission upon exposure to UV light (Ando et al., Proc. Natl. Acad. Sci. USA 99: 12651-12656, 2002). Recently described fluorescent proteins from sea anemones include green and orange fluorescent proteins cloned from Anemonia sulcata (Wiedenmann et al., Proc. Natl. Acad. Sci. U.S.A., 97:14091-14096, 2000), a naturally enhanced green fluorescent protein cloned from the tentacles of Heteractis magnifica (Hongbin et al., Biochem. Biophys. Res. Commun., 301:879-885, 2003), and a generally non fluorescent purple chromoprotein displaying weak red fluorescence cloned from Anemonia sulcata, and a mutant thereof displaying far-red shift emission spectra (595 nm) (Lukyanov et al., J. Biol. Chem., 275: 25879-25882, 2000).

A recently described red fluorescent protein isolated from the sea anemone Entacmaea quadricolor, EqFP611, is a far-red, highly fluorescent protein with a unique co-planar and trans chromophore (Wiedenmann et al., Proc. Natl. Acad. Sci., U.S.A., 99:11646-11651, 2002). The crystal structure of EqFP611 has been solved, and shows conservation of the 11-stranded beta-barrel structure of GFP MMDB Id: 5742 (Petersen et al., J. Biol. Chem., M307896200, 2003).

Still further classes of GFP-like proteins having chromophoric and fluorescent properties have been described. One such group of coral-derived proteins, the pocilloporins, exhibits a broad range of spectral and fluorescent characteristics (PCT App. Pub. WO 00/46233; Dove et al., Coral Reefs 19:197-204, 2001). Recently, the purification and crystallization of the pocilloporin Rtms5 from the reef-building coral Montipora efflorescens has been described (Beddoe et al., Acta Cryst., D59:597-599, 2003). Rtms5 is deep blue in color, yet is weakly fluorescent. However, it has been reported that Rtms5, as well as other chromoproteins with sequence homology to Rtms5, can be interconverted to a far-red fluorescent protein via single amino acid substitutions (Beddoe et al., Acta Cryst., D59:597-599, 2003; Bulina et al., BMC Biochem., 3:7, 2002; Lukyanov et al., J. Biol. Chem., 275: 25879-25882, 2000).

Expression of GFP and GFP-like proteins is compromised in highly acidic environments (i.e., pH=4.0 or less). Likewise, complementation rates (that is, the efficiency with which the fragments of a split fluorescent protein associate and complement each other to yield a fluorescent signal) are generally inefficient under conditions of pH of 6.5 or lower (see U.S. Pat Pub. No. 2005/0221343).

### Split Fluorescent Proteins (SFPs), SFP Tags and SFP Detectors

The embodiments described herein utilize Split Fluorescent Proteins (SFPs). An SFP is a protein complex composed of two or more protein fragments (SFP fragments) that individually are not fluorescent, but, when formed into a complex, result in a functional (that is, fluorescing) fluorescent molecule. Complementary sets of such fragments are also known as a SFP system. Additionally, the embodiments described herein utilize SFP tags and SFP detectors, which are based on a complementary set of SFP fragments. An SFP tag is a SFP fragment that, when fused to a heterologous protein or peptide (i.e., a test protein), allows detection of the heterologous protein using the complementary SFP fragment. The SFP detector is the SFP fragment corresponding to the SFP tag. Thus, an SFP tag and the complementary SFP detector are two complementing fragments of a SFP. Construction of a test protein fused to a SFP tag or SFP detector is typically accomplished via cloning of the nucleic acid encoding the test protein into a nucleic acid construct encoding the SFP tag or SFP detector. SFPs, SFP systems, a number of specifically engineered tag and detector fragments of a SFP, as well as DNA constructs and vectors use thereof are disclosed herein and known to the skilled artisan. See, e.g., U.S. Pat. App. Pub. No. 2005/0221343; Int. Pat. App. Pub. No. WO/2005/074436; Cabantous et al., Nat. Biotechnol., 23:102-107, 2005; Cabantous and Waldo, Nat. Methods, 3:845-854, 2006.

In some embodiments, split-GFP is utilized as the SFP. See, e.g., U.S. Pat. App. Pub. No. 2005/0221343 and Int. Pat. App. Pub. No. WO/2005/074436, hereby incorporated by reference in their entirety; and Cabantous et al., Nat. Biotechnol., 23:102-107, 2005; Cabantous and Waldo, Nat. Methods, 3:845-854, 2006. Split-GFP includes modified fragments of GFP which do not fluoresce on their own, but will fluoresce when in the presence of the remaining fragment or fragments. In the context of split-GFP, a SFP tag is a split-GFP fragment that, when fused to a heterologous protein or peptide (i.e., a test protein), allows detection of the heterologous protein using the complementary split-GFP fragment (i.e., the SFP detector).

In some embodiments, split-GFP includes split-GFP fragments GFP S1-10 and GFP S11. GFP S1-10 corresponds to GFP beta strands 1-10 and GFP S11 corresponds to beta strand 11. Neither molecule fluoresces alone, but will form the complete fluorophore when brought into association. In other embodiments, variations of GFP S1-10, or variations of GFP S11 may be utilized. For example, GFP S1-10 OPT (SEQ ID NO: 4) and GFP S1-10 A4 (SEQ ID NO: 6) may be used as a split-GFP S1-10 fragment. Further, for example, GFP S11 214-238 (SEQ ID NO: 8), GFP S11 214-230 (SEQ ID NO: 10), GFP S11 M1 (SEQ ID NO: 12), GFP S11 M2 (SEQ ID NO: 14), GFP S11 M3 (SEQ ID NO: 16) may be used as a split-GFP S11 fragment. In other examples where split-GFP is utilized, the split GFP may include split-GFP fragments GFP S1-9 and GFP S10-11. GFP S1-9 corresponds to GFP beta strands 1-9 and GFP S10-11 corresponds to beta strands 10-11. Neither molecule fluoresces alone, but will form the complete fluorophore when brought into association. In some embodiments, variations of GFP S1-9, or variations of GFP S10-11 may be utilized. For example, GFP 1-9 OPT (SEQ ID NO: 18) may be used as a split-GFP S1-9 fragment and GFP 10-11 OPT (SEQ ID NO: 19) may be used as a split-GFP S11 fragment. In still other examples where split-GFP is utilized, a tripartite system is used that includes GFP S11, GFP S10 and GFP S1-9.

SFP tags and detectors may be based on any complementary set of SFP fragments. For example, GFP S11 or GFP S10-11 may be SFP tags, in which case GFP S1-9 or GFP S1-10 would be the complementary SFP detectors. Alternatively, in the case where GFP S1-10 is fused to a heterologous protein, GFP S1-10 will be the SFP tag and GFP S11 will be the corresponding SFP detector. In some examples, the SFP tag and SFP detector are based on a circular permutant of a SFP, for example as described herein and in U.S. Pat. App. Pub. No. 2005/0221343 and PCT Pub. No. WO/2005/074436. In the context of a SFP composed of two complementary fragments, wherein the SFP has an 11 beta-strand barrel structure similar to GFP, the SFP tag typically will include one or two strands of the 11 beta-strand barrel structure and the SFP detector typically will include the remaining strands of the 11 beta-strand barrel structure. Typically, when fused to a test protein, a SFP tag is substantially non-perturbing to the structure of the test protein. Small, engineered split fluorescent protein tags can be engineered to be less perturbing to fusion protein folding and solubility relative to the same proteins fused to the full-length fluorescent protein (see, e.g., Cabantous et al., Nat. Biotechnol., 23:102-107, 2005; Pedelacq et al., Nat. Biotechnol., 24:79-88, 2006).

### Making Split Fluorescent Proteins

In addition to known SFPs, SFPs for use in the disclosed methods include SFPs that may be developed using known methods, for example as described herein and in U.S. Pat. App. Pub. No. 2005/0221343 and PCT Pub. No. WO/2005/074436.

In embodiments described herein, any fluorescent protein that has a structure with a root mean square deviation (RMSD) of less than 5 angstroms, often less than 3 or 4 angstroms, and preferably less than 2 angstroms from the 11-stranded beta-barrel structure of MMDB Id:5742 may be used in the development of self-complementing fragments of a SFP. RMSD is the root mean square superposition residual in Angstroms. This number is calculated after optimal superposition of two structures, as the square root of the mean square distances between equivalent C-alpha-atoms. In some cases, fluorescent proteins exist in multimeric form. For example, DsRed is tetrameric (Cotlet et al., Proc. Natl. Acad. Sci. U.S.A., 98:14398-14403, 2001). As will be appreciated by those skilled in the art, structural deviation between such multimeric fluorescent proteins and GFP (a monomer) is evaluated on the basis of the monomeric unit of the structure of the fluorescent protein.

As appreciated by one of ordinary skill in the art, a suitable fluorescent protein structure can be identified using comparison methodology well known in the art. In identifying the protein, a crucial feature in the alignment and comparison to the MMDB ID:5742 structure is the conservation of the beta-barrel structure (i.e., typically including 11 beta strands, but in at least one case, fewer beta strands (see, Wiedenmann et al., Proc. Natl. Acad. Sci. U.S.A., 97:14091-14096, 2000), and the topology or connection order of the secondary structural elements (see, e.g., Ormo et al., Science 273: 5280, 1392-1395, 1996; Yang et al., Nat. Biotechnol. 10:1246-51, 1996). Typically, most of the deviations between a fluorescent protein and the GFP structure are in the length(s) of the connecting strands or linkers between the crucial beta strands (see, for example, the comparison of DsRed and GFP in Yarbrough et al., Proc. Natl. Acad. Sci. U.S.A., 98:462-467, 2001). In Yarbrough et al., alignment of GFP and DsRed is shown pictorially. From the stereo diagram, it is apparent that the 11 beta-strand barrel is rigorously conserved between the two structures. The c-alpha backbones are aligned to within 1 angstrom RMSD over 169 amino acids, although the sequence identity is only 23% comparing DsRed and GFP.

In comparing structure, the two structures to be compared are aligned using algorithms familiar to those in the art, using for example, the CCP4 program suite (Acta Cryst. D50, 760-763, 1994). In using such a program, the user inputs the PDB coordinate files of the two structures to be aligned, and the program generates output coordinates of the atoms of the aligned structures using a rigid body transformation (rotation and translation) to minimize the global differences in position of the atoms in the two structures. The output aligned coordinates for each structure can be visualized separately or as a superposition by readily-available molecular graphics programs such as RASMOL (Sayle and Milner-White, TIBS, 20:374, 1995), or Swiss PDB Viewer (Guex and Peitsch, Protein Data Bank Quarterly Newsletter, 77:7, 1996).

In considering the RMSD, the RMSD value scales with the extent of the structural alignments and this size is taken into consideration when using the RMSD as a descriptor of overall structural similarity. The issue of scaling of RMSD is typically dealt with by including blocks of amino acids that are aligned within a certain threshold. The longer the unbroken block of aligned sequence that satisfies a specified criterion, the better aligned the structures are. In the DsRed example, 164 of the c-alpha carbons can be aligned to within 1 angstrom of the GFP. Typically, users skilled in the art will select a program that can align the two trial structures based on rigid body transformations, for example, as described in Dali et al., J. Mol. Biol., 233:123-138, 1993. The output of the DALI algorithm is blocks of sequence that can be superimposed between two structures using rigid body transformations. Regions with Z-scores at or above a threshold of Z=2 are reported as similar. For each such block, the overall RMSD is reported.

The RMSD of a fluorescent protein for use in the methods described herein is within 5 angstroms of MMDB Id: 5742 structure, which is the GFP structure disclosed by Ormo & Remington, MMDB Id: 5742, in the Molecular Modeling Database (MMDB), PDB Id: 1EMA PDB Authors: M. Ormo & S. J. Remington PDB Deposition: 1 Aug. 1996 PDB Class: Fluorescent Protein PDB Title: Green Fluorescent Protein From Aequorea Victoria. The Protein Data Bank (PDB) reference is Id PDB Id: 1 EMA PDB Authors: M. Ormo & S. J. Remington PDB Deposition: 1 Aug. 1996 PDB Class: Fluorescent Protein PDB Title: Green Fluorescent Protein From Aequorea Victoria. (See, e.g., Ormo et al., Science, 273:1392-5, 1996; Yang et al., Nat. Biotechnol., 10:1246-51, 1996), for at least 80% of the sequence within the 11 beta strands. Preferably, RMSD is within 2 angstroms for at least 90% of the sequence within the 11 beta strands (the beta strands determined by visual inspection of the two aligned structures graphically drawn as superpositions, and comparison with the aligned blocks reported by DALI program output). As appreciated by one of skill in the art, the linkers between the beta strands can vary considerably, and need not be superimposable between structures.

In some examples, the design of a split-fluorescent protein may be briefly illustrated as follows. As will be appreciated by those skilled in the art, the design of a split-chromophoric protein detection system will involve the same steps and principles. First, the fluorescent protein of interest is structurally analyzed in order to determine appropriate splice (or “split”) points for generating individual fragments. As will be understood by those skilled in the art, this may be accomplished by reference to a known crystal structure of the fluorescent protein, either with or without superpositioning with the GFP crystal structure or another fluorescent protein crystal structure, by primary sequence alignment with GFP, by predictive structural modeling (with reference to a known fluorescent protein structure, e.g., GFP), etc.

Appropriate splice (or “split”) points are typically found within the amino acid sequences between beta-sheets of the SFP structure (e.g., if the SFP structure is similar to the eleven beta-barrel structure of GFP), specifically, the loop and turn motifs. In the design of a simple, two-fragment system, a fluorescent protein may be split into two fragments at any point in the molecule between contiguous beta-strands (e.g., within the turn or loop motifs occurring between beta-strands), in order to generate a first fragment corresponding to a first set of contiguous beta-strands, and a second fragment corresponding to a second set of contiguous beta-strands, the total complement of beta-strands being contained within the combination of the two fragments. Thus, for example, one may split the fluorescent protein into fragments corresponding to strands 1-9 and 10-11, or to strands 1-10 and 11. All 11 beta-strands of the fluorescent (or chromophoric) protein are represented in the combination of fragments. It should be noted that circular permutants of a fluorescent protein may also be created, by ligating the native N and C termini and introducing new start and stop codons, and split into fragments corresponding to contiguous beta-strands (for example, into fragments corresponding to pre-permutant strands 9-1 and 2-8). Exemplary two-fragment split-GFP systems are known to the skilled artisan and further described herein.

### V. Optional In Vivo Identification of Soluble Protein and Soluble Protein Complex

Some embodiments optionally include an in vivo split-SFP complementation screen. For example an in vivo split-GFP complementation assay for detection of soluble protein (see, e.g., Cabantous et al., Nat. Biotechnol., 23:102-107, 2005; Cabantous and Waldo, Nat. Methods, 3:845-854, 2006). Combination of the in vivo soluble protein screen with the disclosed in vitro soluble protein screen allows for both in vivo and in vitro detection of a soluble protein or a soluble protein complex in one simple assay. This allows comparison of expression within the host cell of the soluble protein or soluble protein complex with the in vitro solubility of the soluble protein or soluble protein complex.

In some embodiments, host cells are engineered to express both (or all) complementary fragments of a SFP, one or two of which are fused to one or more test proteins. The fragments may be expressed simultaneously or sequentially, depending upon whether the assay aims to detect (and quantify) total protein expression or only soluble and/or insoluble fractions.

In some embodiments, the disclosed methods include quantification of test proteins, and more specifically the quantification of soluble fraction, insoluble fraction, as well as total protein expression. Methods of detecting in vivo expression of protein using a SFP system are known to the skilled artisan. For example, total protein quantity (soluble and insoluble) may be assayed in vivo by co-expression of the test protein-SFP tag fusion and the complementary fragment. The degree of fluorescence is proportional to the quantity of total protein. See, e.g., U.S. Pat. App. Pub. No 20050221343.

In some examples of the optional in vivo soluble protein screen, expression of the test proteins, test protein-SFP tag fusions and SFP fragments or SFP fragment fusion proteins is controlled by an inducible promoter as described herein. For example, cells are induced to simultaneously express the test protein-SFP tag fusions and SFP detector fragments or SFP detector fusion proteins. As described further herein, this allows estimation of the expression of the test protein-SFP tag fusions and SFP detector fragments or SFP detector fusion proteins.

Alternatively, cells are induced to express the test protein-SFP tag fusion for a time sufficient to permit expression of the fusion protein, e.g., in E. coli, typically about 0.5 to 3.5 hours), followed by a “resting” period (approximately 0.5 to 1.5 hours in E. coli) to allow inducing agent to diffuse out of the cells (or, in mammalian cells, by active repression of the promoter, using, for example, anti-sense polynucleotides to shut off the promoter). Cells are then induced to express the SFP detector, typically for about 0.5 to 4 hours in E. coli. An alternative embodiment, for mammalian cells, uses protein transfection to introduce SFP detector proteins after the resting period or following active repression of the first inducible promoter as described herein.

The in vivo solubility assays are amenable to high-throughput screens, as a large number of cells expressing variants of a test protein, fused to the SFP tag, can be assayed for solubility indicated by fluorescence generated from complementation with the SFP detector expressed in or provided to the cells.

In some embodiments (e.g., using mammalian cells), expression of the test protein-SFP tag fusion is turned off after the fusion has been expressed, then expression of the SFP detector is activated.

### VI. Libraries

In some embodiments, the methods described herein include incubating at least two host cells at separately addressable locations on the surface of the hydrogel, wherein each host cell includes a nucleic acid encoding a different test protein (e.g., a test protein fused to a SFP tag and/or an affinity tag). For example, the different test proteins may be members of a library of test proteins, including a library of variants of the same protein.

Any method known in the art for generating a library of mutated proteins or protein variants may be used to generate candidate test proteins. The target protein or polypeptide is usually mutated by mutating the nucleic acid. Techniques for mutagenizing are well known in the art. These include, but are not limited to, such techniques as error-prone PCR, chemical mutagenesis, and cassette mutagenesis. Alternatively, mutator strains of host cells may be employed to add mutational frequency (Greener and Callahan, Strat. Mol. Biol., 7:32, 1995). For example, error-prone PCR (see, e.g., Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999) uses low-fidelity polymerization conditions to introduce a low level of point mutations randomly over a long sequence. Other mutagenesis methods include, for example, recombination (PCT publication WO 98/42727); oligonucleotide-directed mutagenesis (see, e.g., the review in Smith, Ann. Rev. Genet., 19:423-462, 1985); Botstein and Shortle, Science, 229:1193-1201, 1985; Carter, Biochem. J., 237:1-7 1986; Kunkel, “The efficiency of oligonucleotide directed mutagenesis” in Nucleic acids & Molecular Biology, Eckstein and Lilley, eds., Springer Verlag, Berlin, 1987, Zoller and Smith, Method. Enzymol., 100:468-500, 1983; Zoller and Smith, Method. Enzymol., 154:329-350, 1987); phosphothioate-modified DNA mutagenesis (Taylor et al., Nucl. Acids Res., 13:8749-8764, 1985; Taylor et al., Nucl. Acids Res., 13:8765-8787, 1985; Nakamaye and Eckstein, Nucl. Acids Res., 14:9679-9698, 1986; Sayers et al., Nucl. Acids Res., 16:791-802, 1988; Sayers et al., Nucl. Acids Res., 16:803-814, 1988, mutagenesis using uracil-containing templates (Kunkel, Proc. Natl. Acad. Sci. U.S.A., 82:488-492, 1985; Kunkel et al., Method. Enzymol., 154:367-382, 1987); mutagenesis using gapped duplex DNA (Kramer et al., Nucl. Acids Res., 12:9441-9456 1984; Kramer and Fritz, Method. Enzymol., 154:350-367, 1987; Kramer et al., Nucl. Acids Res., 16:7207, 1988); and Fritz et al., Nucl. Acids Res., 16:6987-6999, 1988). Additional methods include point mismatch repair (Kramer et al., Cell, 38:879-887, 1984), mutagenesis using repair-deficient host strains (Carter et al., Nucl. Acids Res., 13:4431-4443, 1985; Carter, Methods Enzymol., 154:382-403, 1987), deletion mutagenesis (Eghtedarzadeh and Henikoff, Nucl. Acids Res., 14:5115, 1986), restriction-selection and restriction-purification (Wells et al., Phil. Trans. R. Soc. Lond., 317:415-423, 1986), mutagenesis by total gene synthesis (Nambiar et al., Science, 223:1299-1301, 1984; Sakamar and Khorana, Nucl. Acids Res., 14:6361-6372, 1988; Wells et al., Gene, 34:315-323, 1985; and Grundstrom et al., Nucl. Acids Res., 13:3305-3316, 1985. Kits for mutagenesis are commercially available (e.g., Bio-Rad, Amersham International). More recent approaches include codon-based mutagenesis, in which entire codons are replaced, thereby increasing the diversity of mutants generated, as exemplified by the RID method described in Murakami et al., Nat. Biotechnol., 20:76-81, 2002.

### XII. Kits

Provided herein are kits useful in conducting the various assays described herein. Kits of the invention may facilitate the use of SFP systems for the identification of soluble proteins and soluble protein complexes as described herein. Various materials and reagents for practicing the methods described herein may be provided. For example, a kit may contain reagents including, without limitation, polypeptides or polynucleotides, cell transformation and transfection reagents, reagents and materials for purifying polynucleotides and polypeptides including lysis regents, protein denaturing and refolding reagents, agar, agarose, affinity reagents, agarose gels, agarose gel that contains an immobilized affinity reagent, as well as other solutions or buffers useful in carrying out the assays and other methods of the invention. Kits may also include control samples, materials useful in calibrating the assays of the invention, and containers, tubes, microtiter plates and the like in which assay reactions may be conducted. Kits may be packaged in containers, which may include compartments for receiving the contents of the kits, instructions for conducting the assays, etc.

For example, a kit may provide one or more SFP fragments as described herein, one or more polynucleotide constructs encoding the one or more SFP fragments, one or more polynucleotide constructs encoding one or more cell affinity tags as described herein, cell strains suitable for propagating the constructs, cells pre-transformed or stably transfected with constructs encoding one or more SFP fragments or affinity tags, agarose hydrogel containing an immobilized affinity reagent and reagents for purification of expressed fusion proteins or nucleotide encoding an expressed fusion protein. For example, a kit may provide a nucleic acid construct encoding a SFP tag and a multiple cloning site adjacent thereto, such that an encoding sequence inserted into the multiple cloning site results in a nucleic acid that encodes a test protein fused with the SFP tag, a hydrogel including an immobilized affinity reagent and instructions for carrying out the methods described herein.

In one embodiment of a kit, the kit includes a nucleic acid construct containing the coding sequence of a SFP tag (e.g., GFP S11) and a multiple cloning site for inserting a test protein in-frame at the N-terminus of the SFP tag coding sequences. Optionally, the insertion site may be followed by the coding sequence of a linker polypeptide in frame with the coding sequence of the downstream SFP tag sequence. A specific embodiment is the pTET-SpecR plasmid as described in U.S. Pat. App. Pub. No. 2005/0221343. This nucleic acid construct is used to produce test protein-SFP tag fusions in suitable host cells. In some embodiments, the kit further contains a pre-purified SFP detector (e.g., GFP 1-10 polypeptide) used to detect the test protein-SFP tag fusions expressed by the recipient construct.

In some embodiments involving in vivo detection of soluble protein, the kit further contains an SFP detector construct which is compatible with the SFP tag construct and encodes the SFP detector under the control of an independently regulated promoter. In an alternate in vivo assay embodiment, cells containing an assay vector (e.g., vector encoding GFP 1-10 under the control of an inducible promoter) are provided in the kit, along with a compatible SFP tag vector into which test proteins may be cloned, wherein expression in controlled by a separately inducible promoter. The cells containing the SFP detector vector may be transformed with the SFP tag vector, and cell fluorescence monitored.

### XIII. Systems

Also provided are systems for conducting the methods described herein. For example, an example system for detecting soluble protein and/or soluble protein complex includes (1) a nucleic acid construct encoding a SFP tag and a multiple cloning site (into which can be inserted, for instance, a sequence encoding a test protein), and (2) a hydrogel including an immobilized affinity reagent. Such systems may also include a nucleic acid construct encoding a SFP detector that is complementary to the SFP tag and/or purified SFP detector protein. Optionally, the SFP detector protein may be provided within the hydrogel or provided separately therefrom. In some embodiments, the systems includes a host cell including the nucleic acid construct encoding the SFP tag and a multiple cloning site, the nucleic acid construct encoding a SFP detector that is complementary to the SFP tag, or both.

In some embodiments, the system includes a nucleic acid construct encoding a SFP tag (e.g., split-GFP S11), an affinity tag and a multiple cloning site, as well as a hydrogel including an immobilized affinity reagent that binds the affinity tag. In yet other embodiments, the system includes a nucleic acid construct encoding a SFP tag and a multiple cloning site, a nucleic acid construct encoding an affinity tag and a multiple cloning site, and a hydrogel including an immobilized affinity reagent that binds the affinity tag. Systems may further include a nucleic acid construct encoding a SFP detector complementary to the SFP tag (e.g., split-GFP S1-10), or the hydrogel may include the SFP detector.

### EXAMPLES

The following examples are provided to illustrate certain particular features and/or embodiments and should not be construed as limiting.

### Example 1

### A High-Throughput Immobilized Bead Screen for Stable Soluble Proteins and Multi-Protein Complexes

This example describes a colony screen for E. coli expressing soluble proteins and assembled multi-protein complexes. Proteins with an N-terminal 6-His tag and C-terminal GFP S11 tag are fluorescently labeled in situ by complementation with a GFP 1-10 detector. After partial colony lysis, soluble proteins diffuse through a supporting filtration membrane and are captured on immobilized affinity beads. The results show the benefit of the approach for screening libraries for soluble proteins and stable complexes. Further, the method may be modified for the use of two orthogonal tags for in vitro capture and detection; thus, leveraging the ‘tandem affinity purification’ concept (Puig et al., Methods, 24:218-229, 2001), and allowing the split-GFP technology to be applied to a broader scope of biological problems, including the identification of stably assembled multiprotein complexes (Rigaut et al., Nat. Biotechnol., 17:1030-1032, 1999).

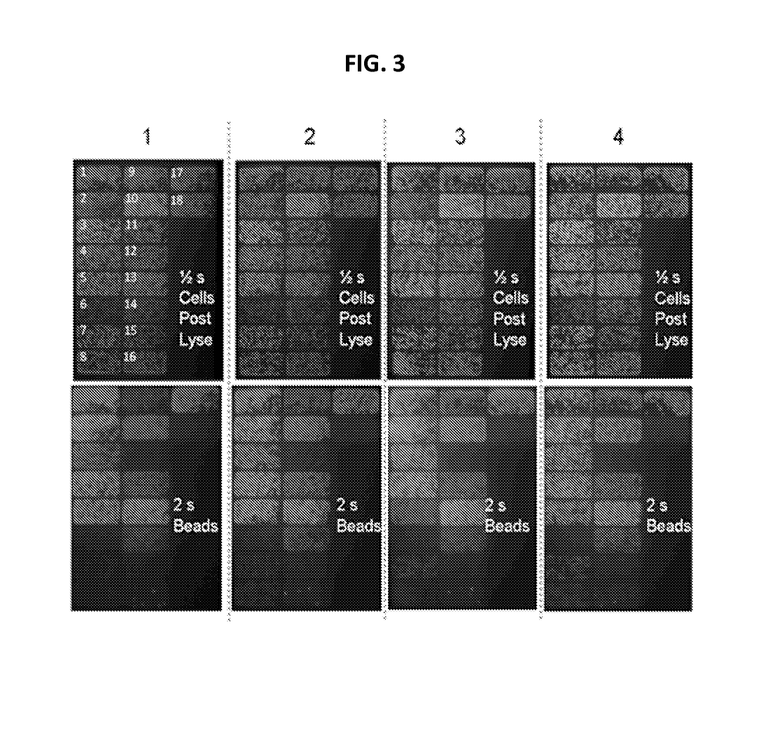

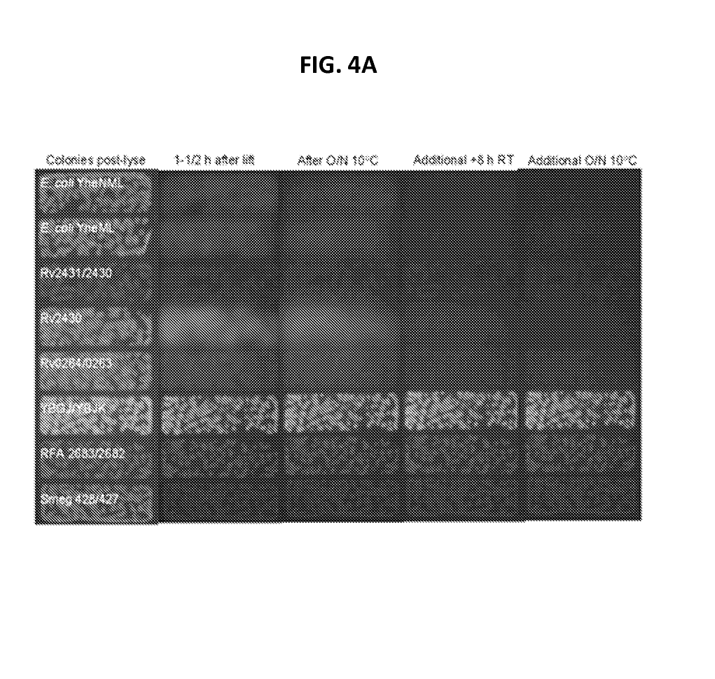

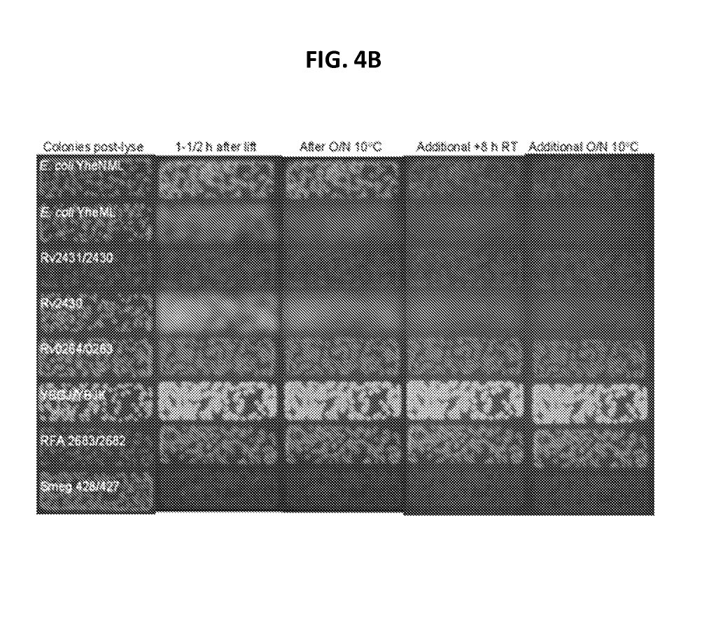

The two-tag, colony based screen described in this example has many advantages over existing colony based screens. Through the use of split-GFP complementation it reports both expression and solubility of tagged proteins without the use of antibodies. The approach maintains colony viability, avoids replica plates, and uses inexpensive, readily available materials. One caveat is the use of chemical lysis. Generic chemical lysis reagents may affect the solubility of proteins, or the stability of protein complexes. Further optimization of the lysis reagent may be necessary (FIG. 3 and FIG. 4). Nevertheless, we envision that this method will be able to be applied to a broad scope of biological problems concerning protein and complex solubility and stability.

### Identification of Soluble Protein

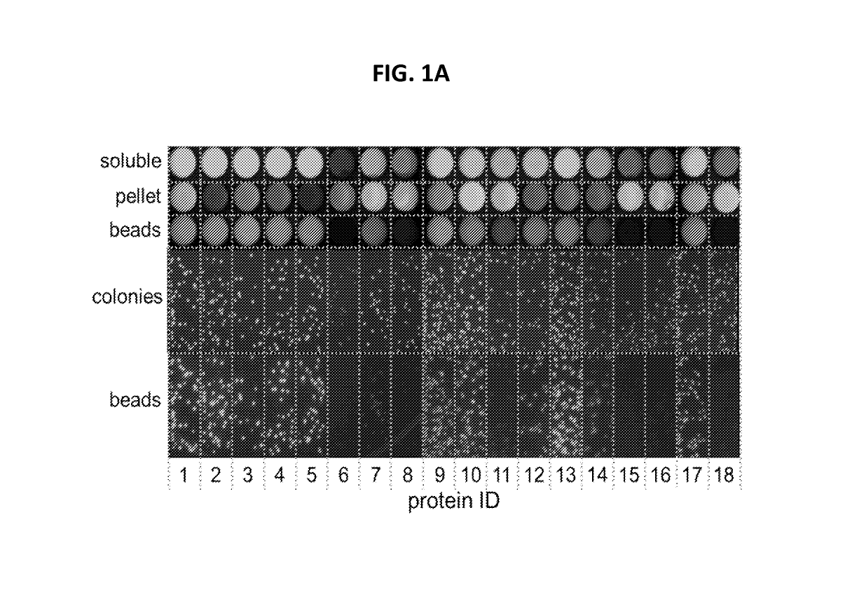

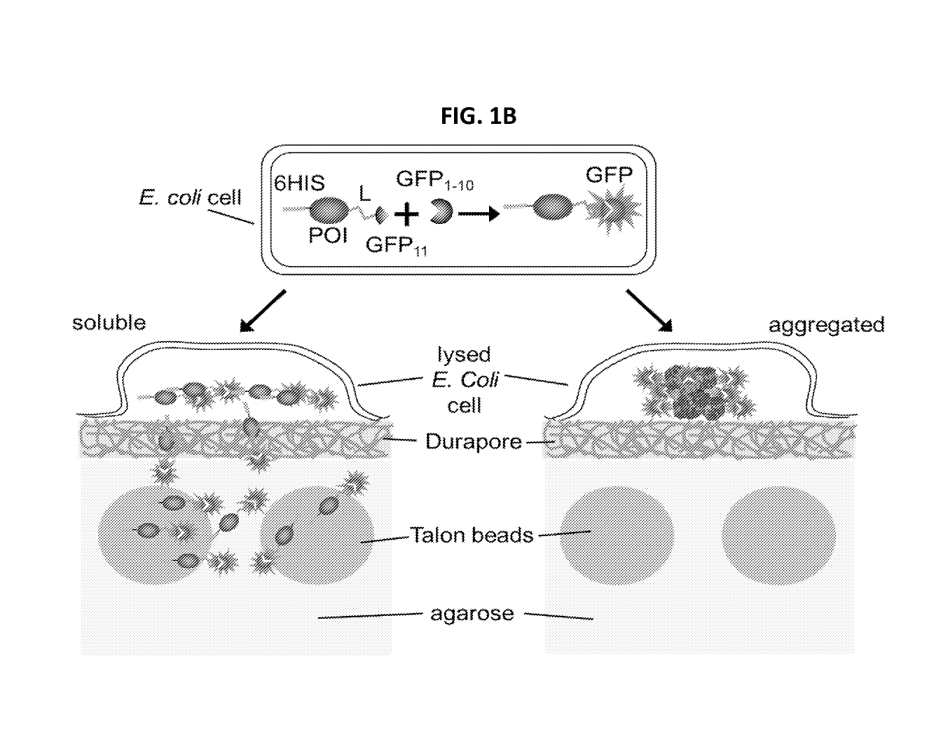

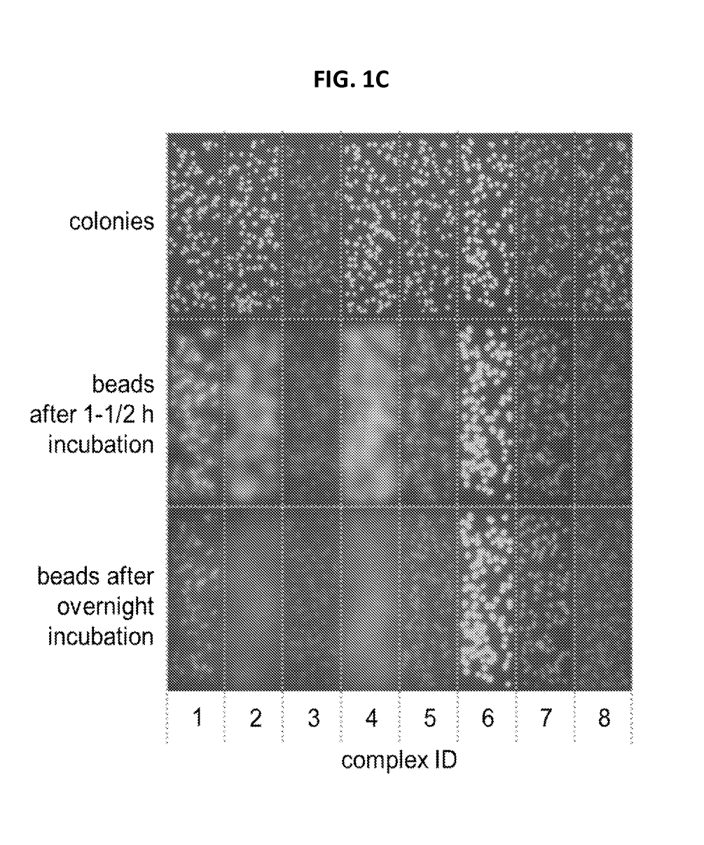

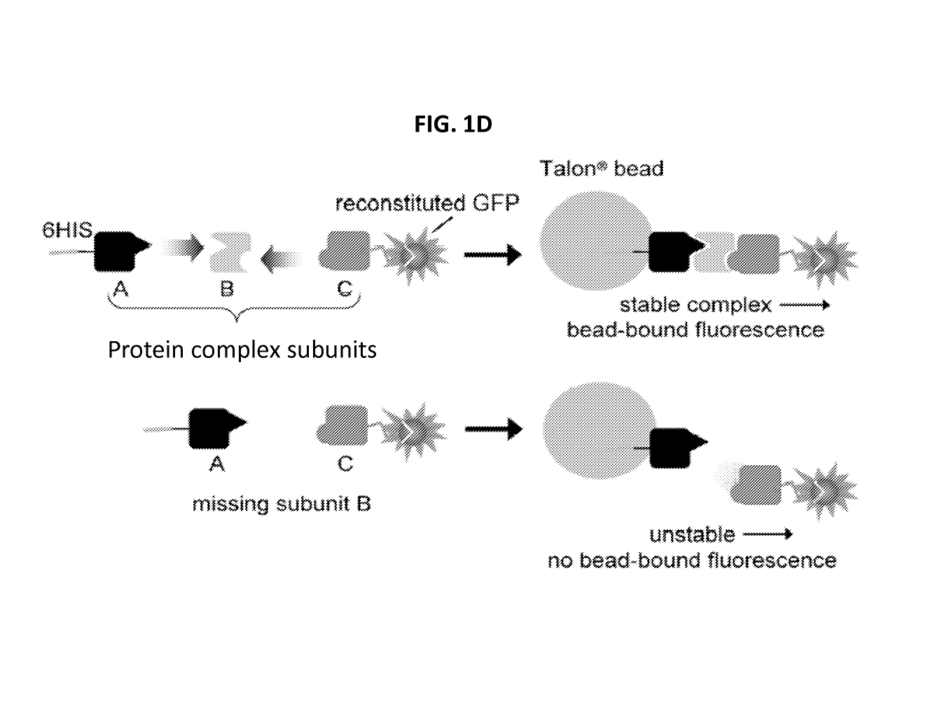

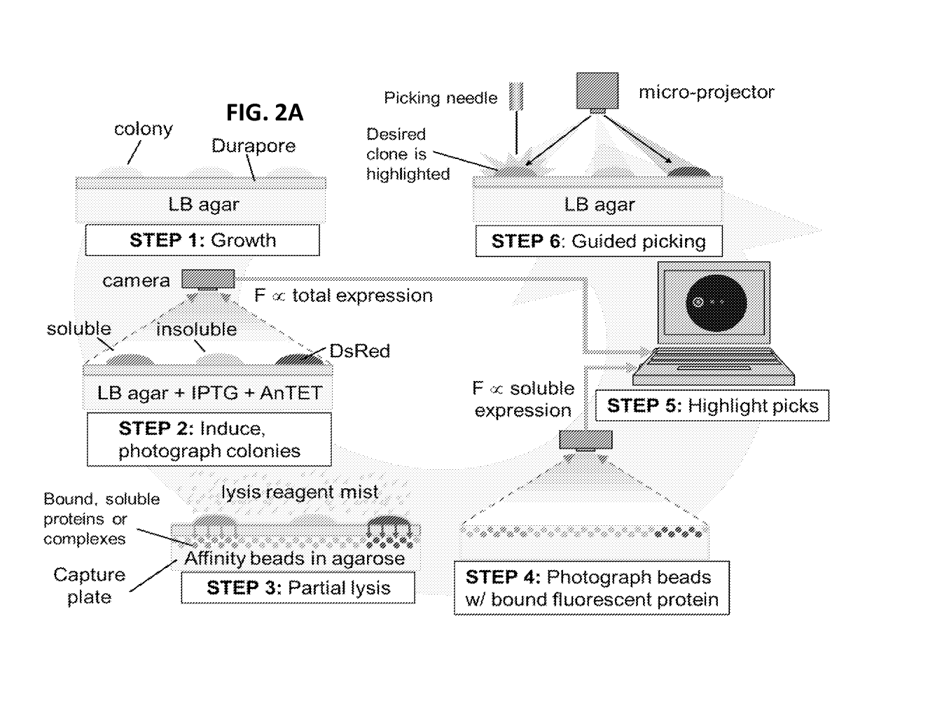

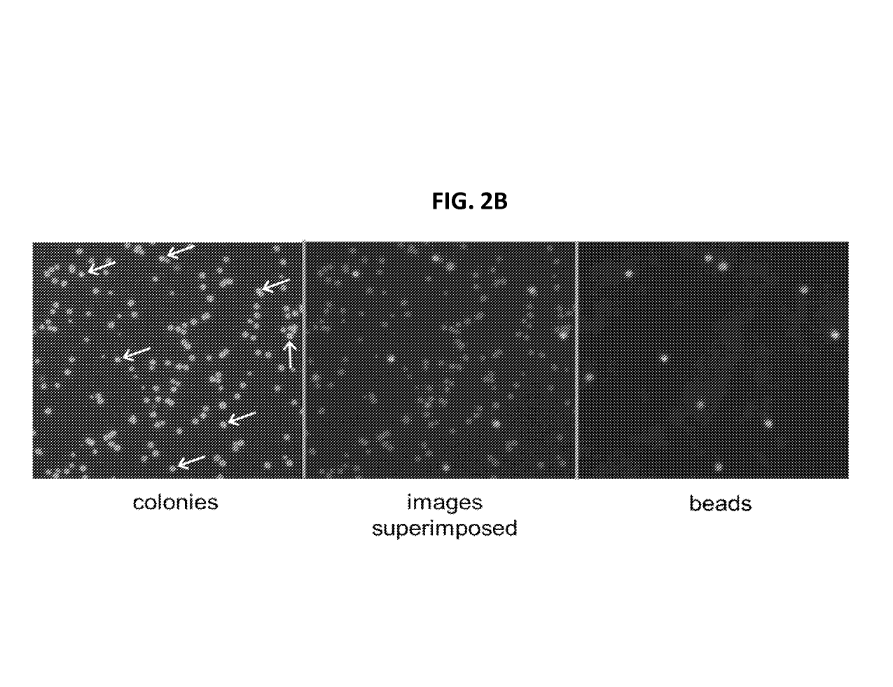

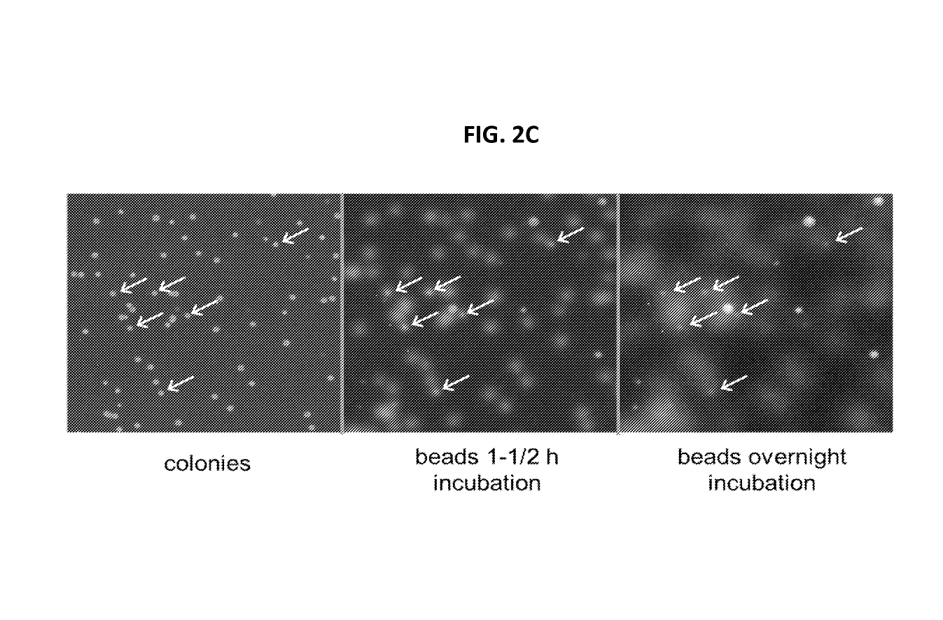

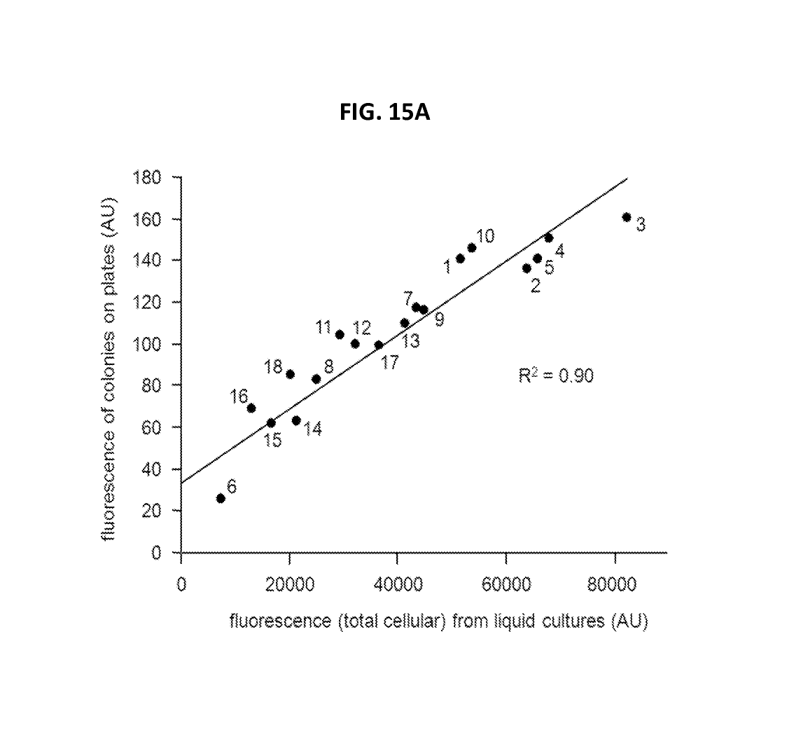

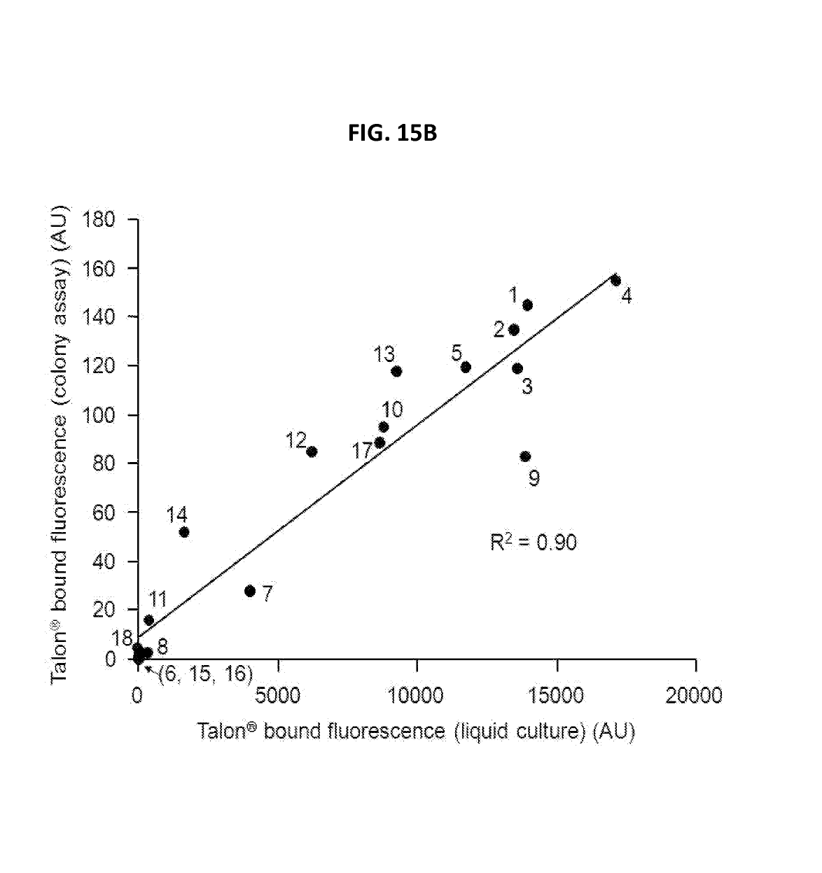

Eighteen test proteins from P. aerophilum (Fitz-Gibbon et al., Extremophiles, 1:36-51, 1997; see Table 1) were first expressed as both liquid cultures (as described herein) and colonies on Durapore® membrane filters (Millipore Co., Billerica, Mass.) resting on LB-agar. In both sets of experiments, co-induction of the GFP S11 tagged protein and GFP S1-10 detector led to rapid in vivo split-GFP complementation with cell fluorescence proportional to total protein expression, as anticipated from previous work (Cabantous et al., Nat. Biotechnol., 23:102-107, 2005; Pedelacq et al., Nat. Biotechnol., 24:79-88, 2006). The samples of the whole cell and Talon® bound fractions from the liquid cultures were photographed (FIG. 1A, upper two rows, Table 1). The immobilized fluorescence of the soluble fraction bound to Talon® metal affinity resin helped to identify all the stable proteins with accessible N-termini (FIG. 1A, top). To evaluate the immobilized bead assay depicted in FIG. 1B, the same co-induction experiment was carried out using single colonies of the 18 control proteins on Durapore® membranes resting on LB agar (FIG. 1A, row marked ‘colonies’). Plotting the fluorescence of the colonies vs. the fluorescence of the liquid culture cell pellet gave a linear correlation coefficient of R²=0.90 (FIG. 15A, Table 1). The co-induced membrane was transferred colony side up to a capture plate containing Talon® metal affinity resin immobilized in agarose (FIG. 1B; see below). The outer surfaces of colonies were partially lysed by misting the membrane with a chemical lysis cocktail (see below). After the released protein was allowed to diffuse through the Durapore® membrane to bind the Talon® metal affinity resin, the membrane with still-viable cells was returned to the LB plate. Fluorescence of the Talon® spots on the capture plate for each control protein (FIG. 1A, lower row marked ‘beads in agarose’) correlated well (R²=0.90) with the fluorescence of the Talon® bound fraction for the same control protein in liquid culture cell lysates (FIG. 1A, upper row marked ‘beads’), giving a linear correlation coefficient R²=0.90 (FIG. 2C). Referring to FIG. 1B, these observations are consistent with the idea that insoluble aggregates of protein are too large to transfer through the Durapore® membrane (Millipore Co., Billerica, Mass.), while stable proteins with accessible 6-His tags will bind to the capture plate. We conclude that colony fluorescence is an acceptable surrogate for total protein expression, and that the immobilized bead assay is well-correlated with a standard assay measuring protein bound to Talon® resin in batch mode.

TABLE 1
18 control proteins from Pyrobaculum aerophilum.

| | | ^{a}Protein expressed alone | | | ^{b}Lysis in TNG buffer | | | |
|---|---|---|---|---|---|---|---|---|
| | | | | ^{j}Fraction | | | ^{j}Fraction | |
| ^{e}# | ^{f}Protein | ^{g,h}Fsol | ^{g,i}Fpel | Soluble | ^{g,k,l}Fsol | ^{g,m,n}Fpel | Soluble | ^{g,o,p}Fbeads |
| 1 | DNA-directed | 18,955 | 4,860 | 0.80 | 41,365 | 16,130 | 0.72 | 17,630 |
| | RNA polymerase | | | | | | | |
| 2 | Sulfite reductase | 26,510 | 0 | 1.00 | 59,730 | 2,860 | 0.95 | 21,005 |
| | (dissimilatory | | | | | | | |
| | subunit) | | | | | | | |
| 3 | c-type | 40,855 | 19,365 | 0.68 | 62,445 | 9,515 | 0.87 | 25,015 |
| | cytochrome | | | | | | | |
| | biogenesis factor | | | | | | | |
| 4 | Translation | 30,615 | 110 | 1.00 | 45,235 | 6,505 | 0.87 | 16,410 |
| | initiation factor | | | | | | | |
| 5 | Ribosomal | 70,700 | 490 | 0.99 | 53,330 | 1,145 | 0.98 | 19,300 |
| | protein S9p | | | | | | | |
| 6 | Polysulfide | 865 | 10,340 | 0.08 | 0 | 5,455 | 0.00 | 0 |
| | reductase subunit | | | | | | | |
| 7 | Nucleoside | 3,415 | 23,345 | 0.13 | 17,035 | 30,640 | 0.36 | 5,685 |
| | diphosphate | | | | | | | |
| | kinase | | | | | | | |
| 8 | Tartrate | | | | | | | |
| | dehydratase β- | 775 | 24,975 | 0.03 | 4,855 | 19,130 | 0.20 | 1,625 |
| | subunit | | | | | | | |
| 9 | 3-hexulose 6- | 31,650 | 515 | 0.98 | 28,785 | 6,730 | 0.81 | 11,620 |
| | phosphate | | | | | | | |
| | synthase | | | | | | | |
| 10 | Hydrogenase | 18,885 | 21,790 | 0.46 | 19,135 | 31,070 | 0.38 | 7,845 |
| | formation hypE | | | | | | | |
| 11 | Methyltransferase | 2,170 | 5,080 | 0.30 | 13,995 | 11,175 | 0.56 | 6,430 |
| 12 | Chorismate | 15,405 | 1,750 | 0.90 | 25,995 | 1,775 | 0.94 | 9,860 |
| | mutase | | | | | | | |
| 13 | Tyrosine t-RNA | 33,395 | 185 | 0.99 | 28,780 | 2,800 | 0.91 | 11,200 |
| | synthetase | | | | | | | |
| 14 | nirD protein | 8,955 | 90 | 0.99 | 13,440 | 3,325 | 0.80 | 3,210 |
| 15 | Soluble | 0 | 3,325 | 0.00 | 755 | 12,745 | 0.06 | 105 |
| | hydrogenase | | | | | | | |
| 16 | Aspartate | 380 | 3,455 | 0.10 | 315 | 14,290 | 0.02 | 85 |
| | aldehyde | | | | | | | |
| | dehydrogenase | | | | | | | |
| 17 | Phosphate | 28,885 | 1,760 | 0.94 | 22,280 | 7,435 | 0.75 | 9,420 |
| | cyclase | | | | | | | |
| 18 | Purine nucleoside | 1,035 | 2,575 | 0.29 | 2,170 | 14,855 | 0.13 | 75 |
| | phosphorylase | | | | | | | |

| | | ^{c}Lysis in Solulyze ® | | | | ^{d}Immobilized bead | |
|---|---|---|---|---|---|---|---|
| | | | | ^{j}Fraction | | assay | |
| ^{e}# | ^{f}Protein | ^{g,k,q}Fsol | ^{g,m,r}Fpel | Soluble | ^{g,o,s}Fbeads | ^{g,t}Colony | ^{g,u}Fbeads |
| 1 | DNA-directed | 47,725 | 4,110 | 0.92 | 14,000 | 147 | 144 |
| | RNA polymerase | | | | | | |
| 2 | Sulfite reductase | 62,890 | 1,320 | 0.98 | 13,485 | 126 | 134 |
| | (dissimilatory | | | | | | |
| | subunit) | | | | | | |
| 3 | c-type | 70,990 | 11,815 | 0.86 | 13,610 | 148 | 118 |
| | cytochrome | | | | | | |
| | biogenesis factor | | | | | | |
| 4 | Translation | 61,900 | 6,450 | 0.91 | 17,170 | 152 | 154 |
| | initiation factor | | | | | | |
| 5 | Ribosomal | 64,215 | 2,000 | 0.97 | 11,750 | 130 | 119 |
| | protein S9p | | | | | | |
| 6 | Polysulfide | 1,115 | 6,145 | 0.15 | 95 | 35 | 2 |
| | reductase subunit | | | | | | |
| 7 | Nucleoside | 18,270 | 25,275 | 0.42 | 4,025 | 123 | 27 |
| | diphosphate | | | | | | |
| | kinase | | | | | | |
| 8 | Tartrate | 3,405 | 21,640 | 0.14 | 345 | 95 | 2 |
| | dehydratase β- | | | | | | |
| | subunit | | | | | | |
| 9 | 3-hexulose 6- | 38,000 | 7,035 | 0.84 | 13,895 | 116 | 82 |
| | phosphate | | | | | | |
| | synthase | | | | | | |
| 10 | Hydrogenase | 29,590 | 24,485 | 0.55 | 8,815 | 149 | 94 |
| | formation hypE | | | | | | |
| 11 | Methyltransferase | 4,435 | 24,925 | 0.15 | 420 | 111 | 15 |
| 12 | Chorismate | 29,165 | 3,090 | 0.90 | 6,255 | 104 | 84 |
| | mutase | | | | | | |
| 13 | Tyrosine t-RNA | 37,735 | 3,810 | 0.91 | 9,285 | 119 | 117 |
| | synthetase | | | | | | |
| 14 | nirD protein | 18,440 | 2,900 | 0.86 | 1,655 | 71 | 51 |
| 15 | Soluble | 1,000 | 15,575 | 0.06 | 25 | 74 | 0 |
| | hydrogenase | | | | | | |
| 16 | Aspartate | 0 | 12,975 | 0.00 | 40 | 77 | 0 |
| | aldehyde | | | | | | |
| | dehydrogenase | | | | | | |
| 17 | Phosphate | 27,560 | 8,930 | 0.76 | 8,675 | 98 | 88 |
| | cyclase | | | | | | |
| 18 | Purine-nucleoside | 3,590 | 16,655 | 0.18 | 0 | 93 | 4 |
| | phosphorylase | | | | | | |

^{a}Protein with GFP S11 tag expressed alone in E. coli liquid shake cultures from the pTET promoter plasmid (Cabantous, et al., Nature Biotechnol., 23:102-107, 2005).
^{b}Tagged protein coexpressed with GFP S1-10 and sonicated in TNG buffer.
^{c}Coexpressed with GFP S1-10 then sonicated in SoluLyse ® buffer as described herein.
^{d}Coexpressed with GFP S1-10 as colonies on membranes as described herein.
^{e}Number of indicated test protein.
^{f}Indicated test protein cloned from Pyrobaculum aerophilum by PCR as previously described (Waldo et al., Nature Biotechnol., 17:691-695, 1999; Cabantous, et al., Nature Biotechnol., 23:102-107, 2005).
^{g}Relative uncertainty of indicated measurement is ~5%.
^{h}Fluorescence of soluble protein measured by plate reader after complementation of soluble fraction by GFP S1-10 in vitro. Background of 1485 subtracted.
ⁱFluorescence after in vitro complementation of urea solubilized pellet fraction using GFP S1-10. Background of 60 subtracted.
^{j}Fraction soluble = Fsol/(Fsol + Fpel) of corresponding fractions.
^{k}Fluorescence of soluble protein measured by plate reader.
^{l}Background of 3790 subtracted.
^{m}Fluorescence of resuspended pellet fraction.
ⁿBackground of 465 subtracted.
^{o}Fluorescence of Talon ® resin beads after binding of indicated soluble fraction in batch mode.
^{p}Background of 785 subtracted.
^{q}Background of 3000 subtracted.
^{r}Background of 460 subtracted.
^{s}Background of 385 subtracted.
^{t}Mean fluorescence of three intact E. coli colonies for indicated construct, measured using a digital camera. Background of 30 subtracted.
^{u}Mean fluorescence of protein bound to Talon ® resin beads, released from corresponding partially lysed colony as described herein. Background of 20 subtracted.

### Identification of Soluble Protein Complex

The assay was also applied to the identification of stable, assembled multiprotein complexes. Trimeric and dimeric complexes were tested, along with their counterparts where one subunit was omitted (Table 2). In all, 8 controls were cloned by PCR as polycistrons from genomic DNA (Table 3). For each multi-protein complex, the first subunit was tagged with the N-terminal 6-His tag, and last subunit with the C-terminal GFP S11 tag. At this point forward, we followed the same basic procedure as for the 18 controls. Clones were first co-induced with the GFP 1-10 detector to label the GFP S11-tagged subunit with fluorescence (FIG. 1C, top). After lysis, we expected all soluble subunits of the complexes would travel through the Durapore® membrane (Millipore Co., Billerica, Mass.) and enter the agarose, regardless of their assembly. On the other hand, only the assembled complexes would decorate the immobilized Talon® metal affinity resin beads with GFP through their N-terminal 6-His tags, forming distinct fluorescent dots under the colonies.

Upon continued incubation soluble, unassembled complex subunits were expected to diffuse throughout the agarose. Photographs were taken after 1½ h at room temperature (FIG. 1C, middle), then after 18 h incubation in the cold room (FIG. 1C, bottom) to observe this diffusion. Referring to FIG. 1C, in agreement with these expectations, bead-bound fluorescence was observed in all fully assembled control complexes including the E. coli trimer YheNML (Numata et al., Structure, 14:357-366, 2006; column 1), the M. tuberculosis heterodimer Rv2431c/Rv2430c (Strong et al., Proc. Natl. Acad. Sci. U.S.A., 103:8060-8065, 2006; column 3), and the heterodimer allophanate hydrolase from M. smegmatis (column 8). Three close homologs of the hydrolase (columns 5-7) also bound beads. Conversely, only diffuse fluorescence was observed in examples where one member of the complex was omitted (columns 2 and 4). As depicted in FIG. 1D, the omission of the YheN subunit from the trimer YheNML (column 2) is hypothesized to destabilize the complex formation, no longer coupling the fluorescent subunit with the bound subunit. The omission of Rv2431c from the dimer Rv2431c/Rv2430c (column 4) may result in an inaccessible 6-His tag, hence the lack of bead-bound fluorescence.

TABLE 2
Multi-protein Complexes, Trial Constructs.

| | Trial | | | |
|---|---|---|---|---|
| # | Construct | Description | Source Organism | Oligomerization |
| 1 | ^{a}Yhe NML | Sulfur | Escherichia coli | ^{a}Trimer |
| | | transfer relay | | |
| 2 | ^{b}Yhe ML | Sulfur | Escherichia coli | ^{b}Unstable |
| | | transfer relay | | (expected) |
| 3 | ^{c}Rv2431c/ | PE/PPE | Mycobacterium | ^{c}Dimer |
| | Rv2430c | | tuberculosis | |
| 4 | ^{d}Rv2430 | PE/PPE | Mycobacterium | ^{d}Unstable |
| | | | tuberculosis | (expected) |
| 5 | ^{e}Rv0264/ | allophanate | Mycobacterium | Dimer |
| | 0263 | hydrolase | tuberculosis | |
| 6 | ^{e}YBGJ/ | allophanate | Escherichia coli | Dimer |
| | YBJK | hydrolase | | |
| 7 | ^{e}RFA 2683/ | allophanate | Rhodopseudomonas | Dimer |
| | 2682 | hydrolase | palustris | |
| 8 | ^{e,f}0428/0427 | allophanate | Mycobacterium | ^{e,f}Dimer |
| | | hydrolase | smegmatis | |

^{a}Tus DCB (Yhe NML) polycistronic trimer recently solved (Numata et al., Structure, 14: 357-366, 2006); positive control.
^{b}Considerable buried surface involved in the contacts between YheN and the adjacent YheM and YheL subunits (Numata et al., Structure, 14: 357-366, 2006), omission of YheN is expected to destabilize the complex. Negative control.
^{c}Bicistronic PE/PPE dimeric complex from M. tuberculosis whose structure was recently solved (Strong, et al., Proc. Natl. Acad. Sci. U.S.A., 103: 8060-8065, 2006).
^{d}There is extensive interaction surface between Rv2431 and Rv2430 (Strong et al., Proc. Natl. Acad. Sci. U.S.A., 103: 8060-8065, 2006). Rv2430 expressed alone fails to bind Talon ® metal affinity resin even though the protein can diffuse in the gel (monitored by GFP fluorescence). Likely the N-terminal 6His tag is inaccessible due to formation of soluble aggregates.
^{e}Predicted bicistronic allophanate hydrolase.
^{f}Protein bicistron recently expressed and purified as dimer; structure solved as stable dimer.

TABLE 3
Primers Used to Amplify Target Multi-protein Complexes.

| | | SEQ |
|---|---|---|
| | Description | ID |
| 5′-3′ Sequence | of primer | NO: |
| AGATATACATATGCGTTTTGCCATCGTGGTGACCGGGCC | Upstream | 20 |
| | YheN NdeI | |
| AATTCGGATCCCCAGGCCATCTGGCTGGAGTGCTTAA | downstream | 21 |
| | YheL | |
| AGATATACATATGAAACGAATTGCGTTTGTTTTTTCTAC | upstream | 22 |
| | YheM | |
| AATTCGGATCCAAATAACATCGTAGTTGGCGAGTTCG | downstream | 23 |
| | YheM | |
| AGATATACATATGTCTTTTGTGATCACAAATCCCGAGGC | upstream | 24 |
| | Rv2431c | |
| AATTCGGATCCAGTGTCTGTACGCGATGACGCCGTGC | downstream | 25 |
| | Rv2430c | |
| AGATATACATATGCATTTCGAAGCGTACCCACCGGAGGT | upstream | 26 |
| | Rv2430c | |
| AGATATACATATGGACGCGGCATTGGCCTGCACCGTGCT | upstream to | 27 |
| | Rv0264c | |
| | M. tb. | |
| AATTCGGATCCCCAGGCTTGTGTTACACCCTGGCCAG | Downstream | 28 |
| | to Rv0263c | |
| | M. tb. | |
| AGATATACATATGCAACGAGCGCGTTGTTATCTGATAGG | Upstream to | 29 |
| | YBGJ of | |
| | YBGJ/YBG | |
| | K E. coli | |
| AATTCGGATCCATTTTCATTGTGCAGCCGCCACGCTA | Downstream | 30 |
| | to YBGK of | |
| | YBGJ/YBG | |
| | K E. coli | |
| AGATATACATATGACCACCCCACCTCCCAGGATGCTGCC | upstream to | 31 |
| | RPA 2683 of | |
| | RPA2683/RP | |
| | A2682 R. palustris | |
| AATTCGGATCCCTCCGCCGCCAGCGCGTCGATTGCCG | downstream | 32 |
| | to RPA 2682 | |
| | of | |
| | RPA2683/RP | |
| | A2682 R. | |
| | palustris | |
| AGATATACATATGACCCTGGGCACCGTCCACAATTACGG | upstream to | 33 |
| | MSMEG042 | |
| | 8 of | |
| | 0428/0427 | |
| | M. smegmatis | |
| AATTCGGATCCGGTCTCGAACGGTCGCCGGGGGTAGG | downstream | 34 |
| | to | |
| | MSMEG042 | |
| | 7 of M. smegmatis | |

### Effect of Co-Induction of S11-Tagged Proteins and GFP 1-10 on Protein Solubility

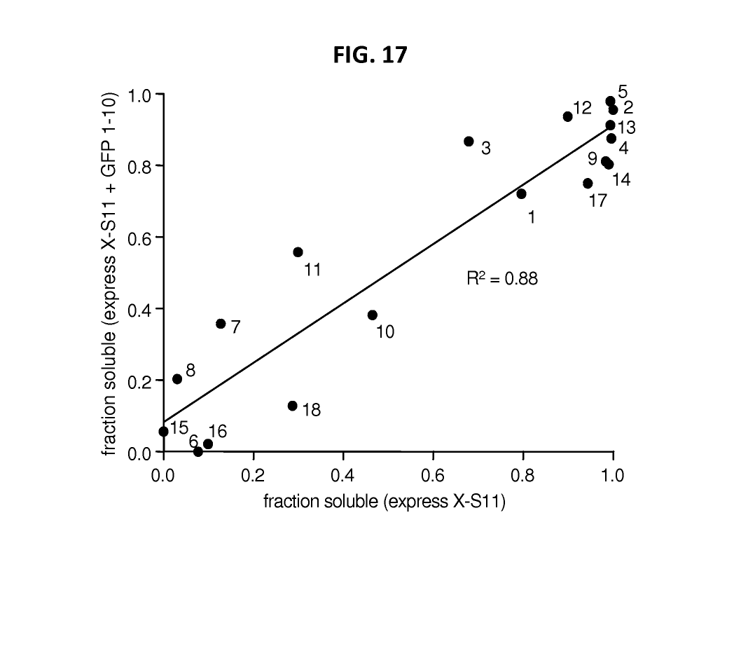

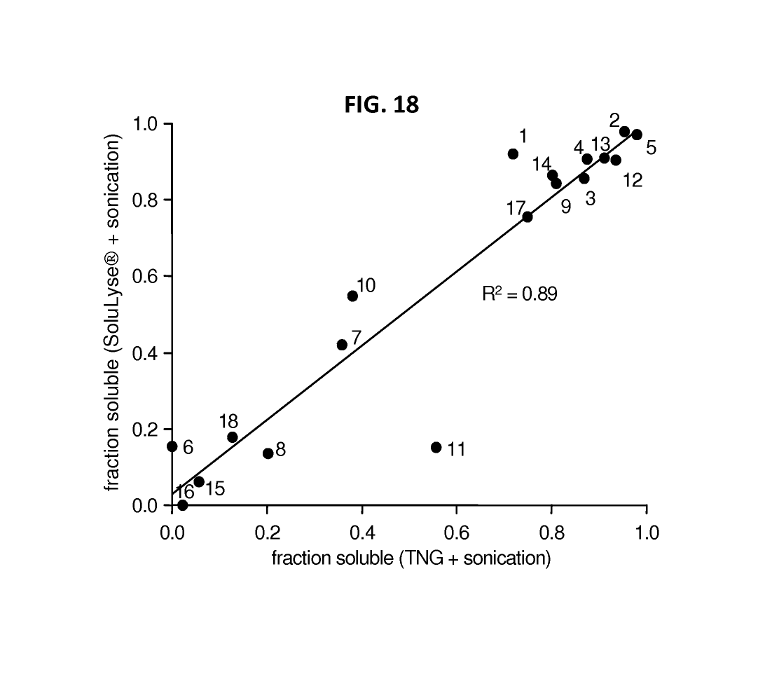

Whole-cell fluorescence is proportional to the expression level of GFP Sit-tagged proteins when they are co-expressed with the GFP 1-10 (Cabantous et al., Nat. Biotech., 23:102-107, 2005). Fusion with intact GFP can substantially reduce protein solubility compared to expressing the proteins alone (Pedelacq et al., Nat. Biotech., 24:779-88, 2006). Since co-expression of the GFP 1-10 and the S11-tagged proteins leads to a fused GFP moiety, we tested if this might perturb protein solubility. In earlier work we lysed cells expressing GFP S11-tagged proteins, then added the GFP 1-10 to the clarified lysates to quantify soluble protein (Cabantous et al., Nat. Biotech., 23:102-107, 2005). We tested whether GFP S11-tagged proteins behave differently after lysis if they had been pre-complemented in the cell with GFP 1-10. To study the effect of co-expression of GFP 1-10 on the solubility of the GFP S11 proteins in greater detail, we performed split GFP assays in vitro on the soluble and pellet fractions of the 18 control proteins listed in Table 1 expressed alone or with GFP S11 tags, as described (Cabantous et al., Nat. Biotech., 23:102-107, 2005; Cabantous and Waldo, Nat. Methods, 3:845-854, 2006), and tabulated the fraction of protein expressed in soluble form (Table 1). In a separate experiment we co-expressed the 18 control proteins with GFP S11 tags along with GFP 1-10 in liquid culture and calculated the fraction of fluorescent protein that was soluble (Table 1). We also measured the Talon® bound fluorescence for later tests as described herein. The fraction of each protein expressed in soluble form was well correlated for the two methods of expression (linear correlation coefficient R²=0.85, FIG. 17). These results indicate that unlike direct fusions to GFP, labeling the proteins with GFP using the split GFP co-expression protocol did not strongly perturb protein solubility. One possibility for this is because the S11 tag had been engineered to not perturb protein solubility and that during co-expression, the S11-tagged protein has a chance to substantially complete its folding on the ribosome prior to interacting with the GFP 1-10 fragment in trans. Furthermore, the GFP 1-10 had also been engineered to not aggregate or complete its folding prior to interacting with the S11 tag, further reducing the likelihood of the GFP 1-10 interfering with the target protein folding. In direct fusions to GFP, the upstream protein can interfere with the folding of the fused GFP domain (Waldo et al., Nat. Biotech., 17:691-695, 1999; Pedelacq et al., Nat. Biotech., 20:927-932, 2002)

### Growth of Liquid Cultures and Assay of Soluble and Insoluble Protein In Vitro

Control proteins were expressed alone and assayed for soluble and insoluble protein in vitro by complementation with the GFP 1-10 reagent (Cabantous et al., Nat. Biotech., 23:102-107, 2005; Cabantous and Waldo, Nat. Methods, 3:845-854, 2006). For co-expression experiments in liquid culture, overnight Luria Bertani (LB) cultures expressing the 18 control proteins were diluted 100-fold into 3.5 ml LB, shaken at 350 RPM for 2 h at 37° C., induced with 350 ng/ml anhydrotetracycline (ANTET) and 1 mM isopropylthiogalactoside (IPTG) for 4 h at 37° C., 350 rpm. Cultures were diluted to 0.4 OD_{600 nm} with LB. 3 ml of each culture was centrifuged for 5 min (16,000×g), the cell pellet was suspended in 200 μl of TNG, and sonicated 3 times for 20 sec (Branson Sonifier, 50% duty cycle, centrifuged 2 min between each sonication cycle). The sonicated samples were centrifuged for 10 min (16,000×g), and the ˜200 μl supernatant (soluble fraction) was recovered by pipetting. The pellet fraction was washed twice with 200 μl of TNG, and resuspended by brief sonication in 200 μl TNG. 40 μl of the soluble or pellet fraction was suspended with 160 μl of TNG and the fluorescence was measured (Cabantous et al., Nat. Biotech., 23:102-107, 2005; Cabantous and Waldo, Nat. Methods, 3:845-854, 2006). In a separate experiment, 40 μl of the remaining soluble fraction was incubated with 40 μl of Talon® (Clontech) (40 μl bed volume) for 10 minutes at 22° C., washed 3 times with 200 μl of TNG, suspended with 160 μl TNG, and the fluorescence was measured (Cabantous et al., Nat. Biotech., 23:102-107, 2005; Cabantous and Waldo, Nat. Methods, 3:845-854, 2006). For tests with Solulyze®, samples were treated as above except 600 μl of the optimized SoluLyse® cocktail (Materials and Methods, Optimization of cell lysis conditions for measuring protein solubility) was used in place of TNG for sonication. For Talon® binding assays, 120 μl samples were used to compensate for the 3-fold dilution caused by the larger lysis buffer volume.

### Colony Induction, Imaging, and Partial Lysis of Clones on Capture Plate

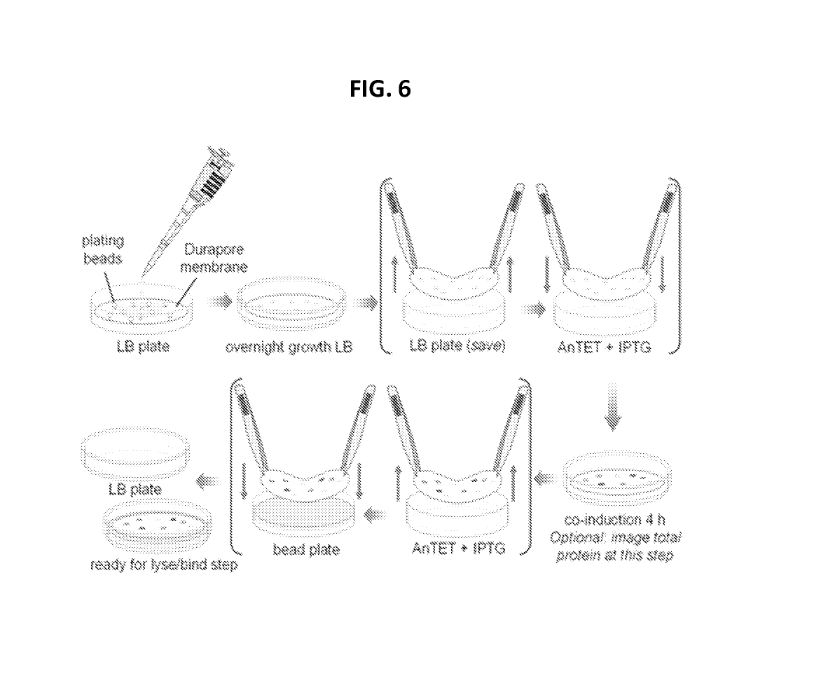

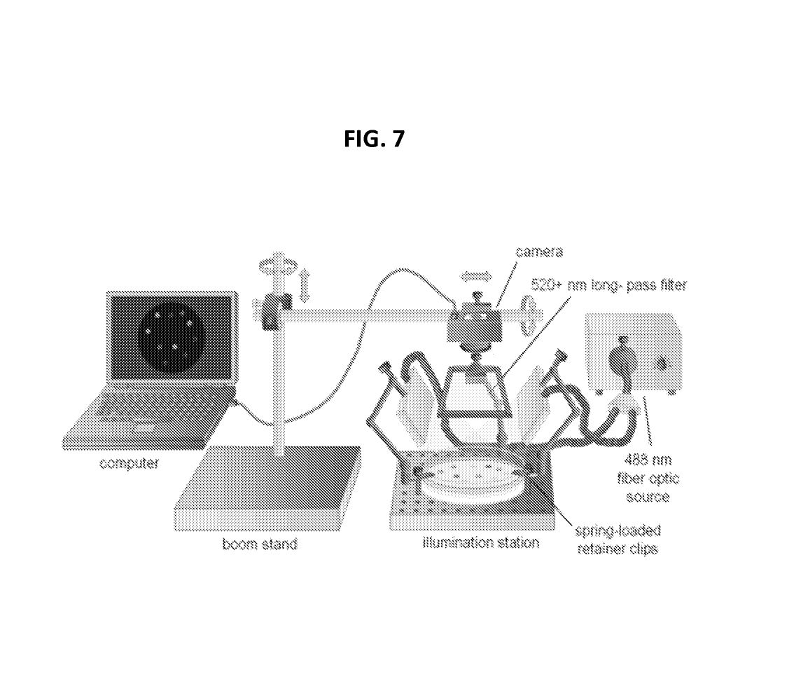

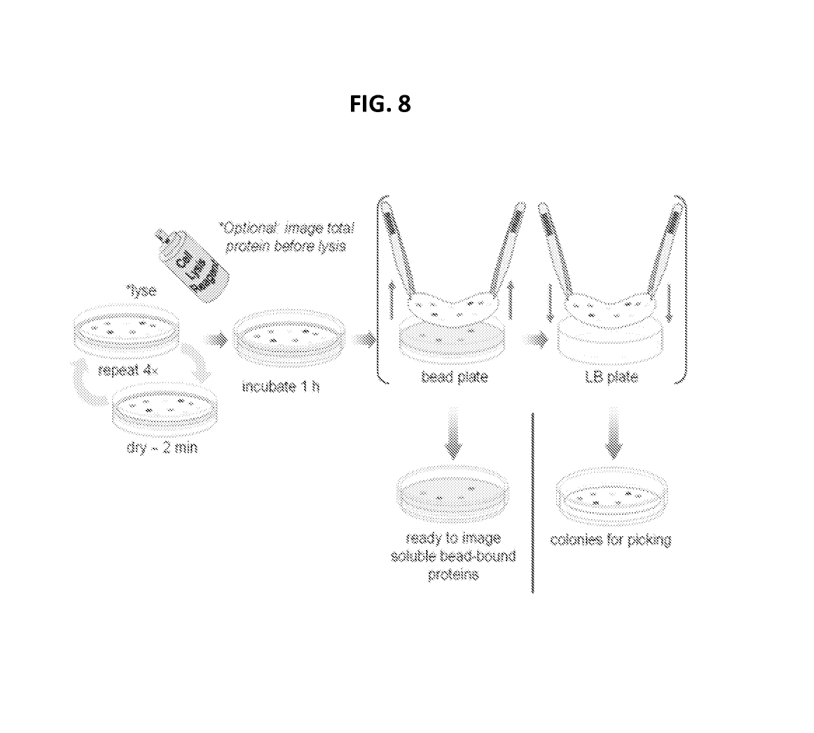

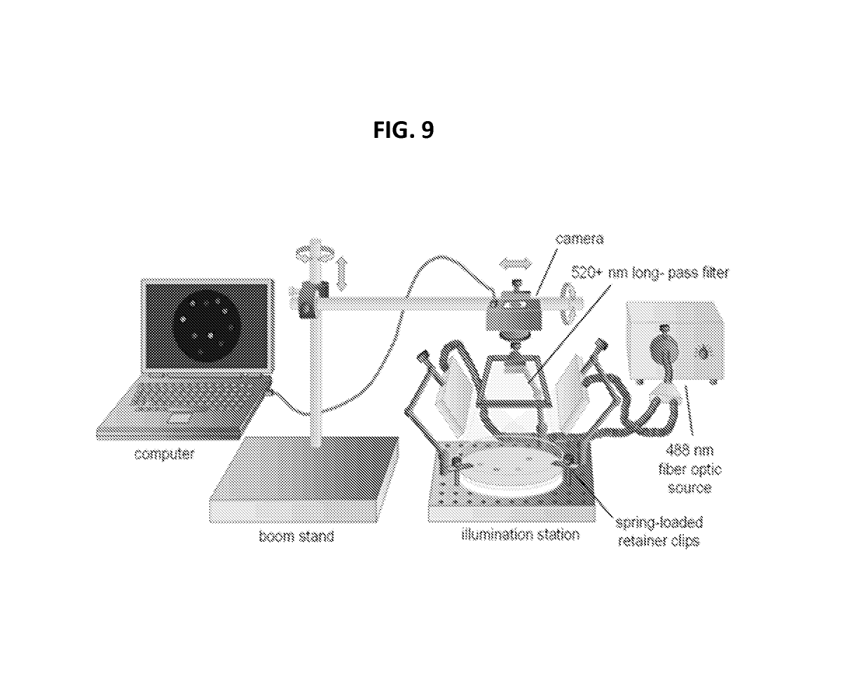

1 OD freezer stocks were serially diluted 350-fold twice in LB media, and plated on Durapore® membrane (Millipore Co., Billerica, Mass.) resting on selective LB media (35 μg/ml kanamycin and 75 μg/ml spectinomycin; FIG. 6). After overnight incubation at 32° C., to induce expression, Durapore® membrane (Millipore Co., Billerica, Mass.) carrying the colonies (ca 1 mm diameter) were transferred face up to LB selective plates containing 350 ng/ml anhydrotetracycline (AnTET) and 1 mM isopropylthiogalactoside (IPTG) and incubated at 37° C. for 4 h (complexes were allowed to incubate an additional 12 h at 10° C.). Durapore® membrane with the induced colonies was moved onto the capture plate and photographed as previously described (Cabantous et al., Nat. Biotechnol., 23:102-107, 2005) using an Illumatool Lighting System (Light Tools Research) to record fluorescence proportional to total protein expression (FIG. 7). A lysis cocktail of 75% v/v Tris SoluLyse® protein extraction reagent (Genlantis, San Diego, Calif.), 1 M Tris-HCl pH 7.5, 15% v/v dH₂O, 2 mM MgCl₂, and 10 AU/ml Benzonase® nuclease (EMD chemicals, Gibbstown, N.J.) was used for the 18 control proteins. 100% Tris SoluLyse® protein extraction reagent (Genlantis, San Diego, Calif.) was used for the eight complexes. A fine-mist spray bottle (Wal-Mart) was filled with ca. 25 ml of the lysis cocktail, sufficient reagent was misted to just wet the surface of the colonies and membrane (ca. 2 ml; FIG. 8), the plate was dried for ˜2 minutes, and the misting cycle was repeated three additional times. The plate was allowed to incubate at 37° C. for 2 hours, and then the Durapore® membrane containing the partially lysed cells was returned to the original LB agar plate (FIG. 8). The capture plate was imaged using the Illumatool system as shown in FIG. 9 and as previously described (Cabantous et al., Nat. Biotechnol., 23:102-107, 2005).

### Guided Colony Picking

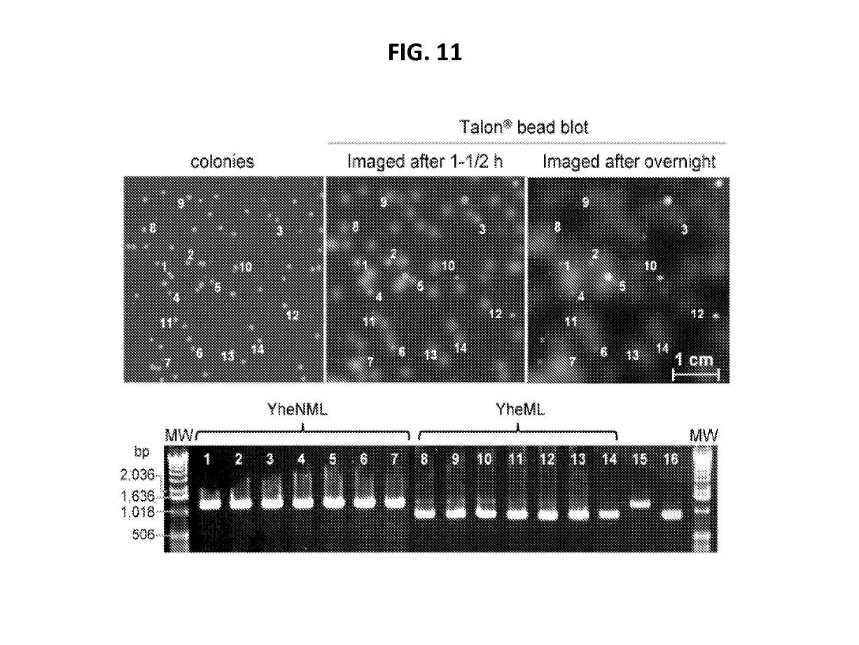

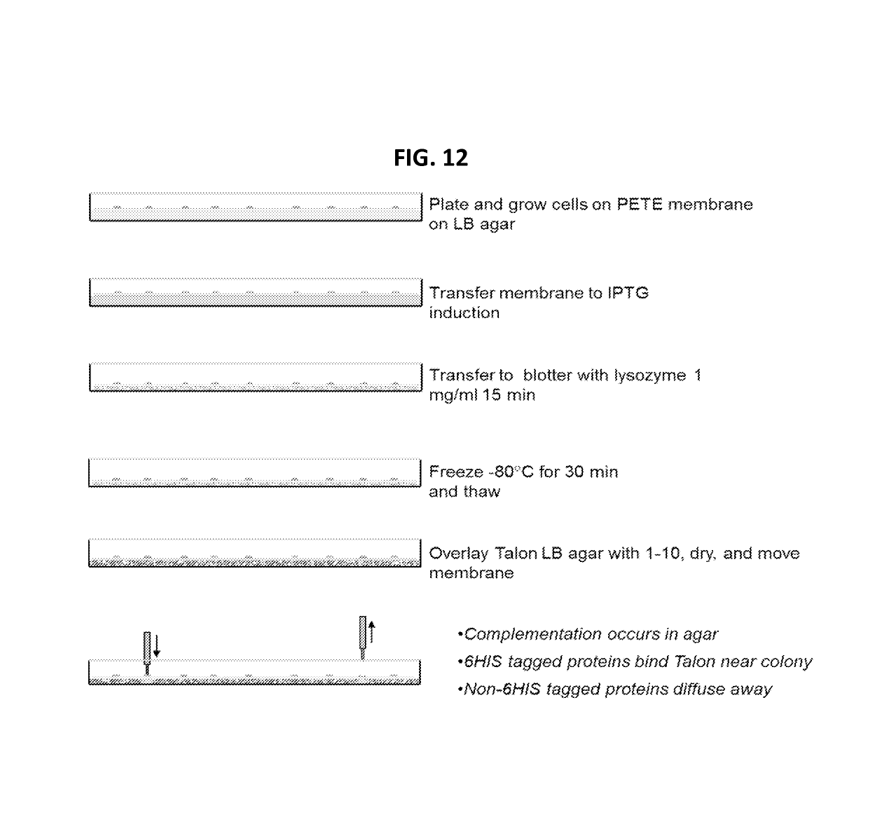

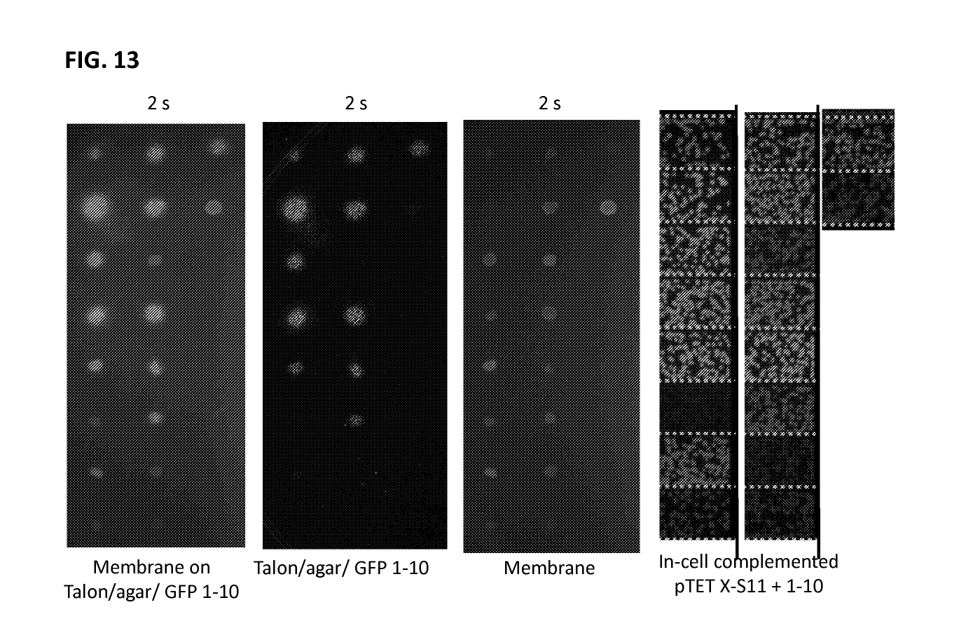

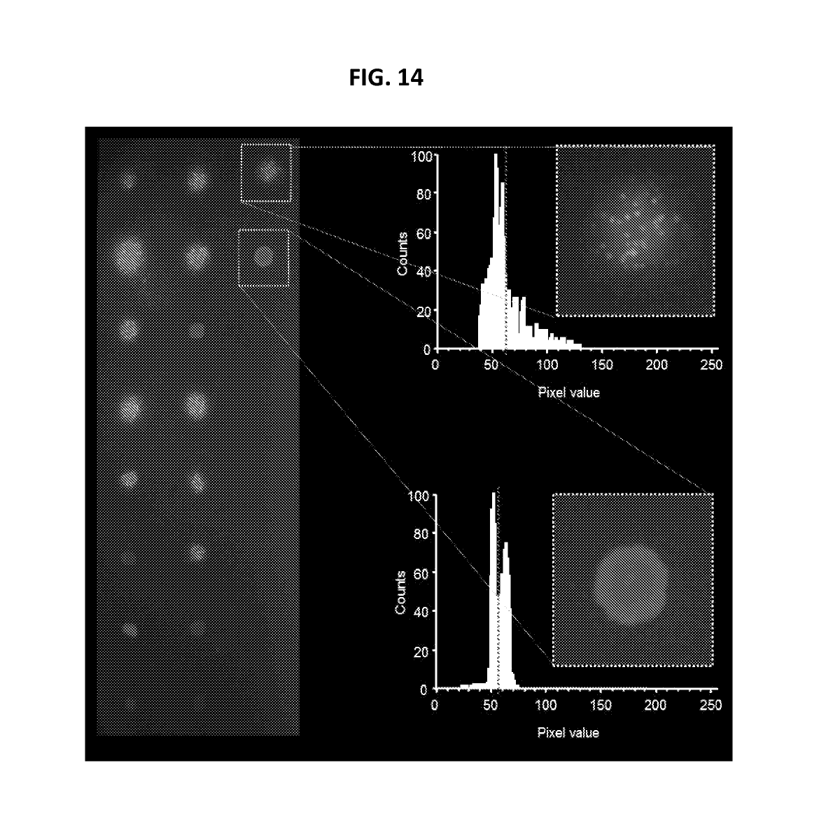

To further validate the method for screening and to demonstrate how the image of the capture plate can be used to guide colony picking as shown in FIG. 2A, mock libraries of the soluble proteins and assembled complexes were constructed. To create a test of the solubility screen, a library was made by mixing E. coli stocks expressing test proteins #17 and #18 in a 1:25 ratio. These test proteins were selected because they are similar in size and in expression level, but differ greatly in solubility (Table 1). A similar expression level of the two test proteins is demonstrated in the fluorescence of the library colonies pre-lysis (FIG. 2B, left). To facilitate the alignment of the capture plate image with the plate containing the still-viable cells, the library was spiked with E. coli expressing a soluble 6-His tagged dsRed protein. Colonies expressing the soluble test protein #17 yielded fluorescent, bead-bound spots on the capture plate (FIG. 2B, right). The capture plate image was projected onto the plate containing the partially lysed colonies, and aligned using the dsRed fluorescent colonies (FIG. 2A, step 6). Green fluorescent spots on the image identified the colonies that were expressing #17 (FIG. 2B, middle). A mock library demonstrating the identification of assembled complexes in a screen was made in a similar manner. E. coli expressing YheNML and YheML were combined and plated in a 1:25 ratio. Following the protocol for the control complexes, colonies were co-induced (FIG. 2C, left), lysed, the capture plates allowed to incubate and photographs were taken at 1½ hours (FIG. 2C, middle) and after overnight incubation (FIG. 2C, right) to allow diffusion of unassembled complexes. The overnight capture plate image was projected and aligned onto the plate containing the colonies. Bead-bound fluorescence identified colonies expressing each assembled complex. Colonies corresponding to 16 compact spots and 10 diffuse spots were picked and grown for plasmid preparation. PCR screens showed that the colonies corresponded to the assembled YheNML or to the unassembled YheML, respectively (FIG. 11).

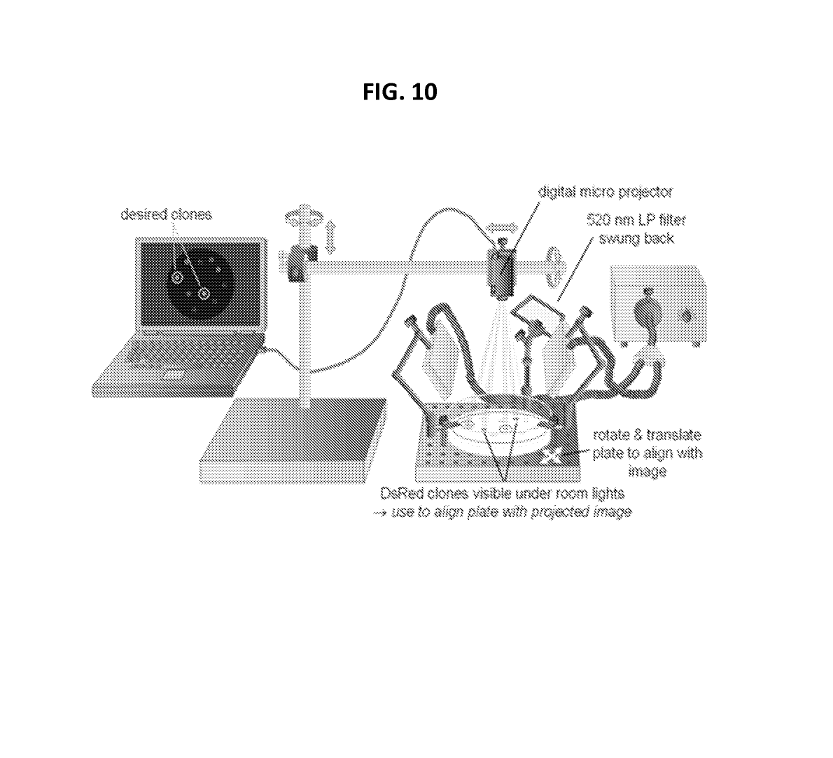

Alignment and picking of colonies proceeded as follows: photographs of colony membranes and Talon® bead capture plates were taken with a digital camera (Olympus Camedia C-5060; (Cabantous et al., Nat. Biotech., 23:102-107, 2005; Cabantous and Waldo, Nat. Methods, 3:845-854, 2006). Images of the colonies and capture plate were manually aligned based on the DsRED spots using the public domain software “HDR Alignment Plug-in” for ImageJ® (Wayne Rasband, NIH). Using the aligned capture plate image, compact fluorescent spots were identified and the corresponding colonies were marked with a white dot in the colony image using Paint Shop Pro® (JASC Software, Corel Corp., Ottawa, Ontario). Marked colonies were given reference numbers by using the object finding function in UTHSCSA Image Tool® 3.0 (Wilcox, Drove, McDavid, and Greer). The highlighted and numbered colony image was then displayed using Paint Shop Pro, and projected onto the original cell colonies using an MPro110® microprojector (3M, Minneapolis, Minn.; FIG. 10). Superimposition of the image and target was optimized by adjusting the projection distance (˜30 cm) and by adjusting the magnification in Paint Shop Pro® (˜34% full-scale setting) using the DsRED colonies as a guide. The desired colonies were easily identified by white spots from the projector. Colonies were picked using sterile toothpicks, and grown in 3 ml of LB and antibiotics overnight at 32° C. Plasmids were prepared using a Qiaprep™ plasmid purification kit (Qiagen, Valencia, Calif.), and 20% v/v glycerol freezer stocks were stored at −80° C. To estimate total expressed protein in colonies and the protein captured on Talon® beads, images of colonies and corresponding Talon® blots were analyzed for average integrated fluorescence using Imagetool® (Scion). For each of the 18 control proteins, 3 colonies of similar size were analyzed and their values were averaged and then compared reader measurements of Talon® batch-binding from liquid culture trials (FIG. 15A, FIG. 15B).

We used the release of fluorescent protein during disruption of colonies by SoluLyse® to estimate that approximately 25% of the colony had been disrupted by the lysis reagent (results not shown). Importantly, the we found that remaining cell mass was viable—a 96 well plate with nutrient media was filled with picks from colonies after lysis and all wells showed excellent growth similar to colonies that had never been lysed. To test whether cross contamination might occur due to the colony lysis, we plated a 50/50 mix of colonies expressing either red fluorescent or green fluorescent proteins at high density (˜3000 colonies on a Bauer plate). Red and green colonies were picked after lysis and streaked out. No red clones were found in green streaks and vice-versa, indicating that there was no cross-contamination (results not shown). We conclude that clones can be recovered without the need for replica plates, a significant advantage for high-throughput protein screens.

### Cloning and Expression of Test Proteins, Complexes, and DsRed Control

Test proteins were cloned into the N-6His pTET S11 SpecR ColE1 ORI vector and transformed into BL21(DE3) bearing the pET GFP 1-10 KanR p15 ORI vector as previously described (Cabantous et al., Nat. Biotechnol., 23:102-107, 2005). Polycistrons encoding test complexes were first amplified by PCR from genomic DNA using the specified primers (Table 3), and then cloned as previously described (Cabantous et al., Nat. Biotechnol., 23:102-107, 2005). Consequently, translation of the first protein in the polycistron began at the vector ribosome binding site; other proteins were translated from the native polycistronic ribosome binding sites (see FIG. 1C). For red fluorescent marker clones, GFP 1-10 was replaced with dsRed (Clontech, Mountain View, Calif.) and co-transformed with the empty pTET-SpecR vector (clones consequently had both KanR and SpecR). Clones were plated, grown, and co-induced as previously described (Cabantous et al., Nat. Biotechnol., 23:102-107, 2005). Hydrophilic Durapore® membrane filter (Millipore Co., Billerica, Mass.; type HVLP; 0.45 μm) was used in place of nitrocellulose to reduce adventitious protein binding during lysis steps in subsequent experiments. 3.5 ml liquid cultures were grown and expressed as previously described (Cabantous et al., Nat. Biotechnol., 23:102-107, 2005), pelleted by centrifugation, and sonicated in 150 μl TNG buffer (0.1 M Tris HCl pH 7.5, 0.15 M NaCl, 10% v/v glycerol) prior to fractionation in to soluble and pellet fractions by centrifugation (16,000 g for 15 min).

### Capture Plates

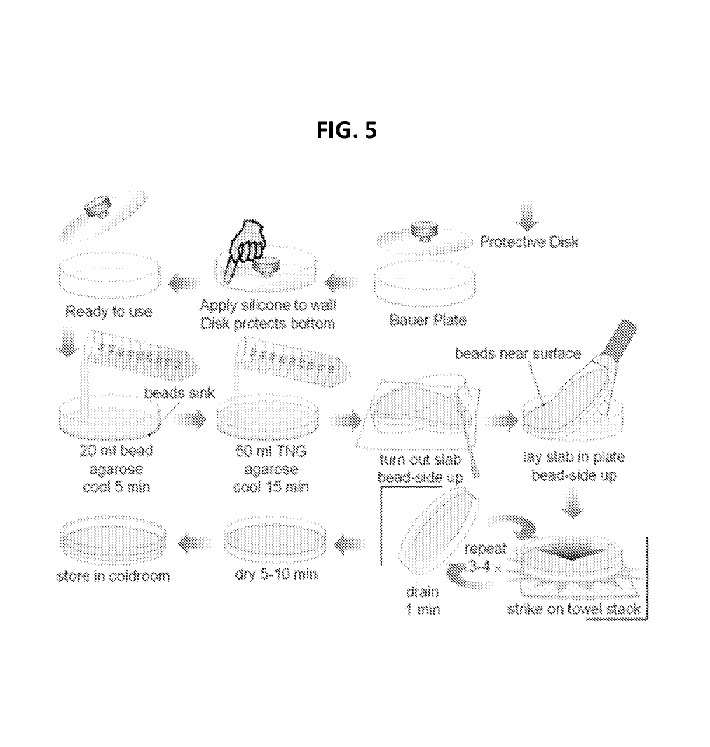

Capture plates were made following the procedure depicted in FIG. 5. Using a gloved finger, silicone vacuum grease (Dow Corning, Midland, Mich.) was applied to the inner walls of two disposable 150 mm Bauer plates dishes (Fisher). As shown in FIG. 5, the interior base of each plate was protected from grease using a custom made protective disc. 30 ml of 50% v/v Talon® metal affinity resin ethanol slurry (Clontech) was placed in a 50 ml Falcon tube and centrifuged at 500 g for 5 min (Beckman J2-21), the supernatant was discarded, and the beads were washed three times with 20 ml of TNG buffer (150 mM Tris-HCl pH 7.5, 0.1 M NaCl, 10% glycerol v/v). The supernatant was discarded, and the 50 ml Falcon tube containing the washed resin bed was preheated in a water bath (80° C., 5 min). 3 g of agarose (Invitrogen, Carlsbad, Calif.) was heated and dissolved in either 200 ml of 150 mM Tris-HCl, pH 7.5 (for control proteins and control protein libraries), or in 200 ml of TNG buffer (for multi-protein complexes). The molten agarose was poured into the Talon® metal affinity resin to a final volume of 50 ml (˜15 ml dry beads, ˜35 ml agarose), gently mixed by inverting the tube, and 25 ml of the suspension poured into the prepared Bauer plates (above). The slabs were misted with ethanol to remove bubbles prior to gelling, solidified by cooling (˜5 min), overlaid with ca. 50 ml of molten agarose in the appropriate buffer (above), then the slabs were cooled to solidify (20 min). The solidified agarose slabs were removed from the plates and placed onto plastic wrap bead-side up; 5 ml of TRIS (0.1 M pH 7.5, 0.15 M NaCl, 10% w/v glycerol was added to the empty Bauer plates, and the agarose slabs were replaced back into the plates bead-side up. The agarose slabs were seated by striking each plate on a paper towel stack. The displaced supernatant was discarded, and the plates were dried for ca. 1 hour in a laminar flow hood prior to wrapping in plastic film for storage (not inverted) at 4° C. for up to 3 days.

### Lysis Conditions

A goal of the approach described herein is to have an assay that yields estimates of solubility that are as close as possible to those obtained by sonication of cells expressing a protein followed by measurement of the amount of the protein that is soluble and insoluble. A series of tests was carried out to identify methods for cell lysis that maximized the correlation between the results of our assays and results from sonication and measurement of fractional solubility of a series of 18 test proteins (Waldo et al., Nat. Biotech., 17:691-695, 1999; Cabantous et al., Nat. Biotech., 23:102-107, 2005; Cabantous and Waldo, Nat. Methods, 3:845-854, 2006). In initial tests, SoluLyse® was used to lyse colonies expressing the 18 control proteins, and TNG was the buffer used in the capture plate (FIG. 3, column 2). Control proteins #7 and #9 were less soluble compared to sonication in (Cabantous et al., Nat. Biotech., 23:102-107, 2005; Cabantous and Waldo, Nat. Methods, 3:845-854, 2006). Next we tested 4 additional conditions: two commercial lysis buffers, SoluLyse® (Genlantis) or Bugbuster® (Novagen EMD); and two capture plate buffers (TNG or Tris HCl) (FIG. 3). The solubility of control protein #5 decreased compared to sonication when lysed with Bugbuster® and captured using Talon® plates with Tris HCl. Control proteins #7 and #9 behaved most similarly to sonication when lysed with SoluLyse® in Tris HCl and captured using Talon® plates with Tris HCl (FIG. 3, columns 3, 4).

To study the effects of the chemical composition of the lysis buffer, a partial factorial screen (Table 4) of 5 chemical lysis components was tested on cell pellets from liquid cultures of test protein #9. This protein showed the greatest sensitivity in preliminary tests on the 18 control proteins (FIG. 3). Parameters included SoluLyse® protein extraction reagent (Genlantis, San Diego, Calif.) (75% or 35% v/v), NaCl (0.15 M), Tris HCl pH 7.5 (0.15 M), glycerol (10% v/v), and 10 AU/ml Benzonase® nuclease (EMD chemicals, Gibbstown, N.J.) with 2 mM of the cofactor MgCl₂, yielding a partial factorial screen with 32 conditions. A 200 μl aliquot of each lysis cocktail (Table 4) was placed in a well of a 96 well PCR plate. Three 3.5 ml cultures of #9 were grown and co-induced as described herein and pooled. 200 μl aliquots were placed in 32 wells of a second 96-well PCR plate, centrifuged, then the pellets were lysed by adding the cocktail plate. Then the plate was processed and the fluorescence of the soluble and pellet fractions measured using a plate reader as previously described Listwan et al., J Struct. Funct. Genomics, 10:47-55, 2009), and the factors that resulted in solubility most similar to sonication were identified by principle component analysis Armstrong et al., Protein Sci., 8:1475-1483, 1999). Benzonase® nuclease (EMD chemicals, Gibbstown, N.J.) and MgCl₂ strongly increased Talon® metal affinity resin-bound soluble protein, whereas NaCl and glycerol decreased it (see Table 4). For protein #9, the optimal buffer included SoluLyse® 75% v/v protein extraction reagent (Genlantis, San Diego, Calif.) Tris, 100 mM Tris HCl pH 7.5, 10 AU/ml Benzonase® nuclease (EMD chemicals, Gibbstown, N.J.) and 2 mM MgCl₂. Using this cocktail, the Talon® metal affinity resin-bound fluorescent protein from the in vitro colony lysis for the 18 controls showed excellent correlation with the amount of Talon® metal affinity resin-bound protein from liquid cultures sonicated in TNG buffer (FIG. 1A).

TABLE 4
Partial Factorial Screen for Lysis Adjuvants.

| | ^{a}% v/v | | | ^{a}% v/v | ^{a,b}Benzonase ® | |
|---|---|---|---|---|---|---|
| Solution | SoluLyse ® | ^{a}[NaCl] | ^{a}[TRIS] | Glycerol | (AU/ml) | |
| # | ^{d}+12 | ^{d}−49 | ^{d}+16 | ^{d}−23 | ^{d}+68 | ^{c}Fluorescence |
| 1 | 75 | 0 | 0 | 0 | 0 | 2143 |
| 2 | 75 | 0 | 0 | 10 | 0 | 1934 |
| 3 | 75 | 0 | 0.1 | 0 | 0 | 2432 |
| 4 | 75 | 0 | 0.1 | 10 | 0 | 2555 |
| 5 | 75 | 0.15 | 0 | 0 | 0 | 2746 |
| 6 | 75 | 0.15 | 0 | 10 | 0 | 2318 |
| 7 | 75 | 0.15 | 0.1 | 0 | 0 | 2569 |
| 8 | 75 | 0.15 | 0.1 | 10 | 0 | 1936 |
| 9 | 38 | 0 | 0 | 0 | 0 | 2097 |
| 10 | 38 | 0 | 0 | 10 | 0 | 1871 |
| 11 | 38 | 0 | 0.1 | 0 | 0 | 2827 |
| 12 | 38 | 0 | 0.1 | 10 | 0 | 2628 |
| 13 | 38 | 0.15 | 0 | 0 | 0 | 2469 |
| 14 | 38 | 0.15 | 0 | 10 | 0 | 2551 |
| 15 | 38 | 0.15 | 0.1 | 0 | 0 | 2587 |
| 16 | 38 | 0.15 | 0.1 | 10 | 0 | 1699 |
| 17 | 75 | 0 | 0 | 0 | 10 | 6770 |
| 18 | 75 | 0 | 0 | 10 | 10 | 7570 |
| 19 | 75 | 0 | 0.1 | 0 | 10 | 7111 |
| 20 | 75 | 0 | 0.1 | 10 | 10 | 5792 |
| 21 | 75 | 0.15 | 0 | 0 | 10 | 4185 |
| 22 | 75 | 0.15 | 0 | 10 | 10 | 3405 |
| 23 | 75 | 0.15 | 0.1 | 0 | 10 | 3643 |
| 24 | 75 | 0.15 | 0.1 | 10 | 10 | 2603 |
| 25 | 38 | 0 | 0 | 0 | 10 | 3421 |
| 26 | 38 | 0 | 0 | 10 | 10 | 2966 |
| 27 | 38 | 0 | 0.1 | 0 | 10 | 10000 |
| 28 | 38 | 0 | 0.1 | 10 | 10 | 6302 |
| 29 | 38 | 0.15 | 0 | 0 | 10 | 3421 |
| 30 | 38 | 0.15 | 0 | 10 | 10 | 2837 |
| 31 | 38 | 0.15 | 0.1 | 0 | 10 | 3285 |
| 32 | 38 | 0.15 | 0.1 | 10 | 10 | 2457 |

^{a}Chemical composition of each of the 32 trial lysis cocktails.
^{b}Solutions containing Benzonase ® nuclease (EMD chemicals, Gibbstown, NJ) were also supplemented with 2 mM MgCl₂.
^{c}Fluorescence (arbitrary units) of GFP-complemented bead-bound fraction protein #9 after cell lysis by indicated solution. Average of 2 replicates, relative uncertainty ca. 5%.
^{d}Principal factors. Positive values indicate component increases fluorescence; negative values indicate component decreases fluorescence (i.e., bead bound protein).

Two aspects of the method that underwent a great deal of refinement were the lysis cocktail and capture plate buffer. In initial trials, 100% SoluLyse® protein extraction reagent (Genlantis, San Diego, Calif.) was used to lyse the cells, and 100 mM Tris pH 7.5, 150 mM NaCl, and 10% glycerol v/v (TNG) was the buffer used in the capture plate (FIG. 3, column 2). While most of the test proteins gave solubility results consistent with previous characterization (Cabantous et al., Nat. Biotechnol., 23:102-107, 2005) and the sonicated liquid culture assay (as described herein), #7 and #9 both appeared to be more insoluble than previous data suggested. A series of trials was set up to compare two commercial lysis buffers, SoluLyse® protein extraction reagent (Genlantis, San Diego, Calif.) and BugBuster® Protein extraction reagent (EMD chemicals, Gibbstown, N.J.) and two capture plate buffer conditions (TNG and Tris-HCH (FIG. 3). Test protein #9's solubility was improved by the presence of Tris-HCl in the capture plates in both the case of BugBuster® Protein extraction reagent (EMD chemicals, Gibbstown, N.J.) and SoluLyse® protein extraction reagent (Genlantis, San Diego, Calif.) (FIG. 3, columns 3, 4). The BugBuster® Protein extraction reagent (EMD chemicals, Gibbstown, N.J.)/Tris-HCl seemed to decrease the solubility of test protein #5. Additionally, test protein #7 was slightly more soluble in the SoluLyse® protein extraction reagent (Genlantis, San Diego, Calif.)/Tris HCl condition, making it the best condition to pursue.

Next, a series of chemical adjuvants were tested in different combinations on test protein #9 (Table 4) to determine whether the presence of NaCl, glycerol, or the presence of both perturbed the solubility of #9 post-lysis. Because #7 and #9, the problem test proteins, are both likely nucleic acid binding proteins according to annotation, Benzonase® nuclease (EMD chemicals, Gibbstown, N.J.) and its cofactor Mg²⁺ were also included in the test. All possible combinations of these adjuvants were tested in an array by a liquid handling robot as described herein. The combination of NaCl and of glycerol seemed to decrease the solubility of #9, while the addition of Benzonase® nuclease (EMD chemicals, Gibbstown, N.J.) and MgCl₂ increased solubility. This was also true for test protein #7 (FIG. 1B). Consequently, a chemical lysis cocktail consisting of SoluLyse® protein extraction reagent (Genlantis, San Diego, Calif.), 100 mM Tris HCl, 10 AU/ml Benzonase® nuclease (EMD chemicals, Gibbstown, N.J.), and 0.2 mM MgCl₂ was used to carry out the solubility screens presented in the main body (FIG. 1B, 2b).

The protein complex assembly screen was first tested on capture plates made with Tris-HCl. The YheNML (Numata et al., Structure, 14:357-366, 2006), Rv2431c/2430c (Strong et al., Proc. Natl. Acad. Sci. U.S.A., 103:8060-8065, 2006) protein complexes and the heterodimer allophanate hydrolase from M. smegmatis Rv0264/Rv0263 were previously reported as stable in vitro. The presence of Tris alone in the agarose seemed inadequate to stabilize the fully assembled complexes once they were lysed from the cells (YheNML, Rv2431c/2430c, and Rv0264/0263) (FIG. 4A). A screen was performed including TNG in the capture plate. The presence of NaCl and glycerol stabilized the fully assembled complexes in vitro. YheNML, Rv2431c/2430c, and Rv0264/0263 yielded bound fluorescent spots (FIG. 4B). The inclusion of NaCl and glycerol in the capture plates appeared to stabilize these complexes relative to Tris HCl alone.

### PCR Verification of Picked Target Control Proteins and Complexes.

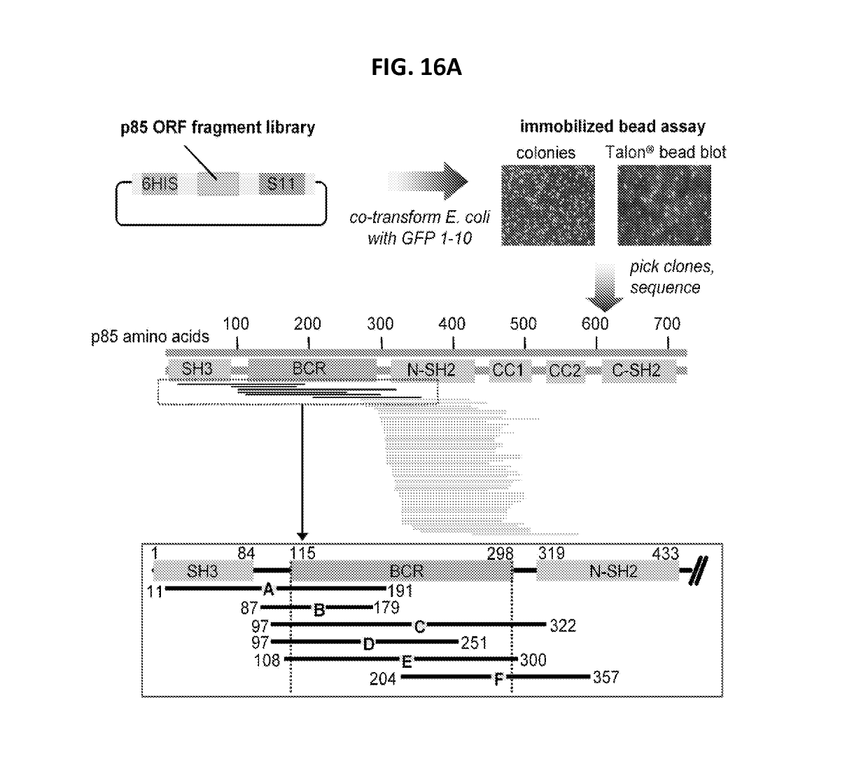

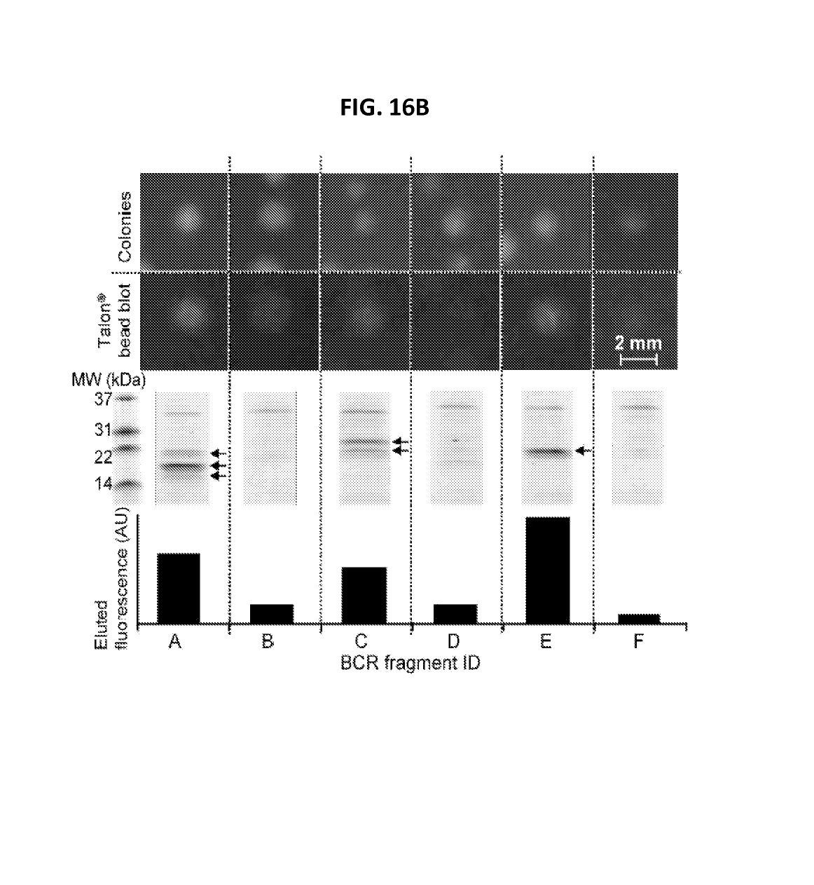

Forward primer #0421007 (TAGAGATACTGAGCACATCAGCAGGACGCACTGACC; SEQ ID NO: 35) and reverse primer #02129811 (GAGGCCTCTAGAGGTTATGCTAGTTATTGC; SEQ ID NO: 36) priming just outside the cloning site of the N6His pTET S11 vector (Cabantous et al., Nat. Biotech., 23:102-107, 2005; Cabantous and Waldo, Nat. Methods, 3:845-854, 2006) were used to amplify control proteins and complexes picked from libraries. Clones used to make the mock libraries were used as size standards. Amplified PCR products were resolved by agarose gel electrophoresis (Cabantous et al., Nat. Biotech., 23:102-107, 2005) with a GelDoc® System (BioRad Laboratories, Hercules, Calif.; see FIG. 11). p85 library optima were sequenced by fluorescent dye terminator sequencing using the vector specific primers described above. Sequences were blasted locally against the p85 gene using Bioedit® 7.0.5 (Hall, Nucleic Acids. Symp. Ser., 41:95-98, 1999). The fragment endpoints were analyzed in Microsoft Excel to make the gene maps presented in FIG. 16A.

### Recycling Metal Affinity Resin

The bead-agarose slab was peeled away from the agarose layer. Eight bead slabs were combined, placed in 1.5 l of dH₂O, then heated to 95° C. in a microwave until the agarose dissolved and most of the beads settled (ca. 20 min). The supernatant was discarded and the remaining suspension of concentrated beads (ca. 200 ml) was poured into a 0.2 μm vacuum filter flask. Beads were washed 3 times with 50 ml of imidazole (500 mM) and then 3 times with 50 ml TNG. The washed beads were suspended in TNG (50% v/v slurry) and stored at 4° C. for further use.

### Example 2

### Isolation of p85 Soluble Protein

This example illustrates the use of the colony screen described in Example 1 for the identification of soluble protein fragments of the H. sapiens phosphoinositide-3-kinase, p85.

### p85 Large Fragment Library, ORF Selection, and Cloning

The H. sapiens phosphoinositide-3-kinase, p85 open reading frame (ORF) was amplified by PCR from plasmid PIK3R1, NM_{—}181523, (OriGene™, Rockville, Md.), digested with DNAse-I, resolved by agarose gel electrophoresis and DNA fragments of the desired size isolated (QiaQuick™ PCR cleanup kit, Qiagen™, Valencia, Calif.). The DNA fragment library was blunt cloned into an internal permissive site of E. coli dihydrofolate reductase (DHFR), and plated on agar plates with 6 μg/ml trimethoprim (TMP) to remove fragments with stop codons according to standard techniques (see, e.g., U.S. Pat. No. 7,390,640; see also, (Lutz et al., Protein Eng., 15:1015-1030, 2002; (Zacchi, et al., Genome Res., 13:980-990, 2003). ORFs were subcloned into the N6His pTET S11 vector and transformed into chemically competent BL21 (DE3), pET GFP 1-10 cells as described (Cabantous et al., Nat. Biotech., 23:102-107, 2005; Cabantous and Waldo, Nat. Methods, 3:845-854, 2006).

### Quantification of Fluorescence of Colonies and Talon® Blots for the p85 Large Fragment Library

Bauer plates with ˜2×10³ colonies were grown overnight at 32° C., then lysed and the bead assay performed as described (Materials and Methods, Colony induction, imaging, and partial lysis of clones on capture plate). A mask was made in Paint ShopPro® and overlaid digitally onto the colony and Talon® blot images so that only the selected targets were visible. The average integrated fluorescence was tabulated for each colony and corresponding Talon® blots in the masked images using the object analysis function in ImageTool®, exported to Microsoft Excel®, and the ends of the corresponding sequenced ORF fragment used to make the fragment map (FIG. 16A).

### Analysis of Solubility of Selected BCR Fragments Containing Clones in Liquid Culture

N6His pTET S11 plasmids with target genes for BCR hits identified from the p85 fragment library screen were isolated by retransforming the 400-fold diluted plasmids and selecting only on spectinomycin. 3.5 ml cultures were grown to 0.5 OD_{600 nm} as described herein (see Growth of liquid cultures and assay of soluble and insoluble protein in vitro section above; see also Cabantous et al., Nat. Biotech., 23:102-107, 2005; Cabantous and Waldo, Nat. Methods, 3:845-854, 2006). Cultures were split into two equal aliquots and cell pellets were frozen overnight at −80° C. An aliquot was thawed, and 500 μl of SoluLyse® was added, mixed, and cells were allowed to incubate at 37° C. for 1 hour prior to measurement of soluble and insoluble protein using GFP 1-10 complementation according to standard techniques (Cabantous et al., Nat. Biotech., 23:102-107, 2005). 80 μl of the complemented, soluble fraction was then incubated with 80 μl resin bed of Talon® resin for 10 minutes at 22° C., and washed with three aliquots of 200 μl SoluLyse® cocktail, prior to reading on a plate reader (Cabantous et al., Nat. Biotech., 23:102-107, 2005; Cabantous and Waldo, Nat. Methods, 3:845-854, 2006). SDS-PAGE and densitometry analyses of soluble, insoluble, and Talon® bound fractions (without the added GFP 1-10) were performed according to standard techniques (Cabantous et al., Nat. Biotech., 23:102-107, 2005; Cabantous and Waldo, Nat. Methods, 3:845-854, 2006).

### Application of the Colony-Based ‘Immobilized Bead’ Assay to Screening an ORF Fragment Library for Soluble Proteins

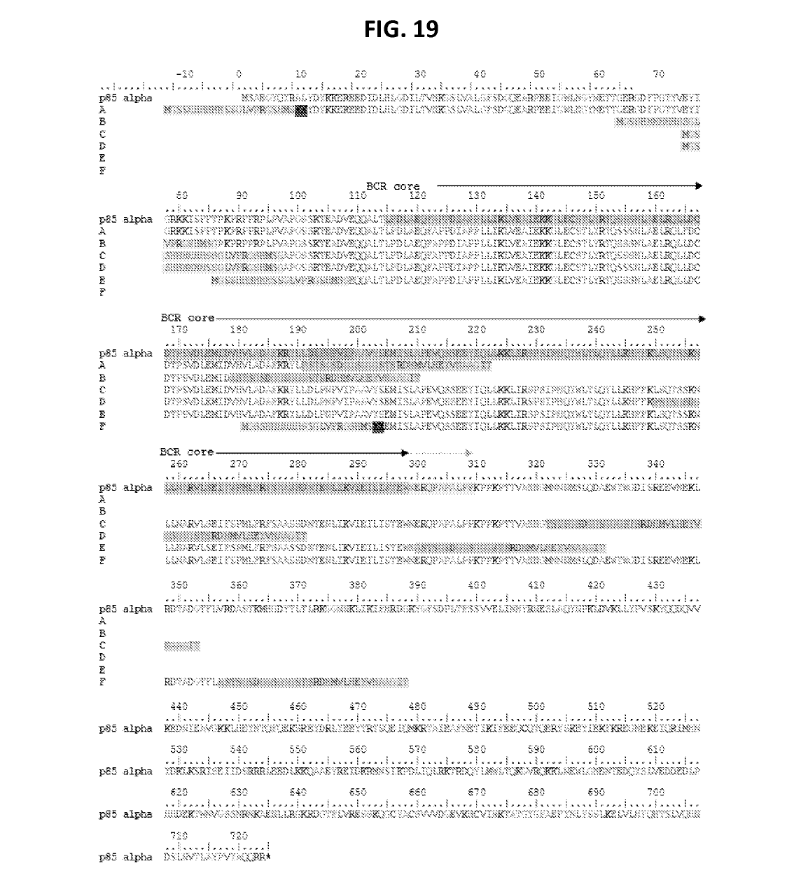

The utility of the soluble protein assay described herein was tested by applying it to the problem of identifying soluble fragments of a large insoluble protein. An open reading frame (ORF) fragment library of the large multi-domain H. sapiens p85 protein was prepared. The soluble protein assay described herein was used to identify soluble fragments in this same ORF library (FIG. 16A). A sample of the library was spiked with clones expressing DsRED to facilitate alignment of the colony plate and capture plate images for analysis and optima selection. Approximately 8,000 clones from the ORF selection library were screened. After photographing the colonies and the Talon® resin plate, we used PaintShop Pro® (JASC) to align the images in a stack for analysis based on the position of the randomly placed DsRED clones. We chose 96 colonies, preferentially picking those with Talon®-bound fluorescence, but also including some with little or no soluble fluorescence. Clones showing no colony fluorescence were avoided. Sequencing of these 96 colonies revealed most contained the central NSH2 domain and some flanking regions (FIG. 16A), and a few contained part or all of the BCR domain. Many of the NSH2 containing constructs appeared to be better expressed than the other domains (data not shown). We suspect this increased expression level may have led to enrichment for NSH2 during the ORF selection (Materials and Methods, p85 large fragment library, ORF selection, and cloning). Since, shorter fragments near the expected size of the NSH2 domain were deliberately not included, most picks containing NSH2 were longer than the hypothetical boundaries. Since our objective focused on the BCR domain, these NSH2 clones were not pursued further. Six clones (A-F; FIGS. 16a, 16b, 19) contained at least half of the BCR domain, and two of these contained the full length BCR domain (C and E; FIGS. 16a, 19). Clone E is the most compact construct that also contains the entire BCR domain.

To test how well the immobilized bead assay predicted success in expression of soluble protein in liquid culture, plasmids for the BCR fragments (clones A through F) were isolated and retransformed without the GFP 1-10 (See Analysis of solubility of selected BCR fragments containing clones in liquid culture above), expressed, and the Talon®-bound fractions visualized by SDS gel electrophoresis (Cabantous et al., Nat. Biotech., 23:102-107, 2005; FIG. 16B middle and FIG. 16B legend). To more precisely measure the soluble BCR protein in each sample, refolded GFP 1-10 was added to the soluble lysate (Cabantous et al., Nat. Biotech., 23:102-107, 2005), and then the fluorescent complemented proteins were bound to Talon®. The fluorescence was measured on a plate reader (FIG. 16B, bottom). The amount of protein as indicated by the Talon® bound fluorescence of the immobilized bead assay (FIG. 16B, top) was well correlated with the liquid culture Talon® bound protein as visualized by SDS-PAGE (FIG. 4B, middle) and in vitro complementation fluorescence (FIG. 16B, bottom). Clones B, D and F were each very faint in the colony-based immobilized bead assay, and are not visible on SDS-PAGE as expected (FIG. 16B). Clones A, C, and E showed up brightly on the immobilized bead assay, were readily visible on SDS-PAGE, and complemented GFP 1-10 well (FIG. 16B, bottom).

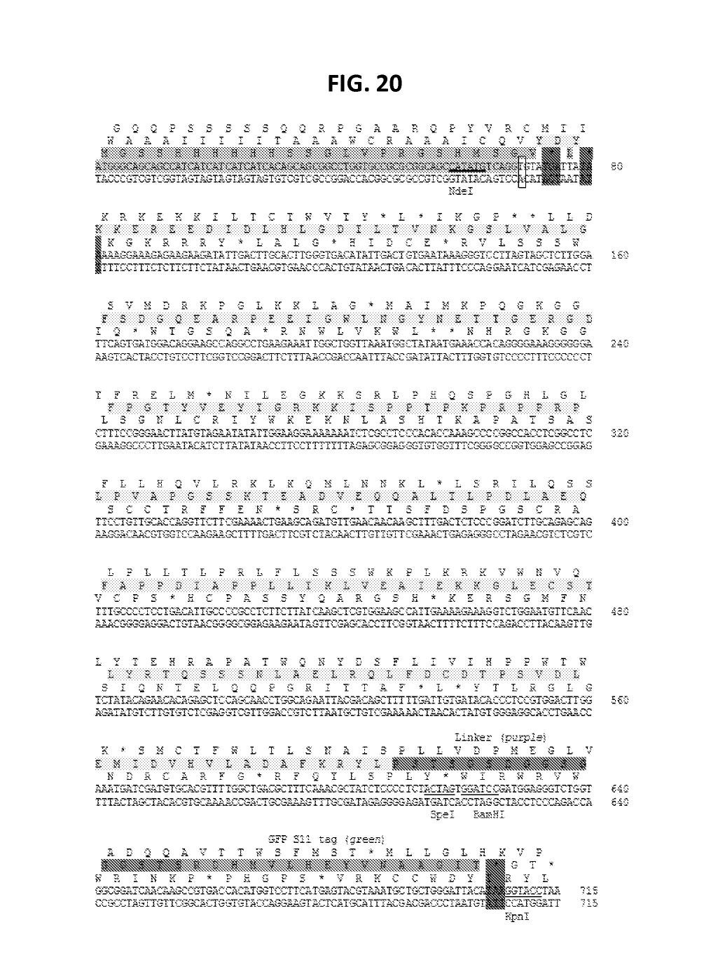

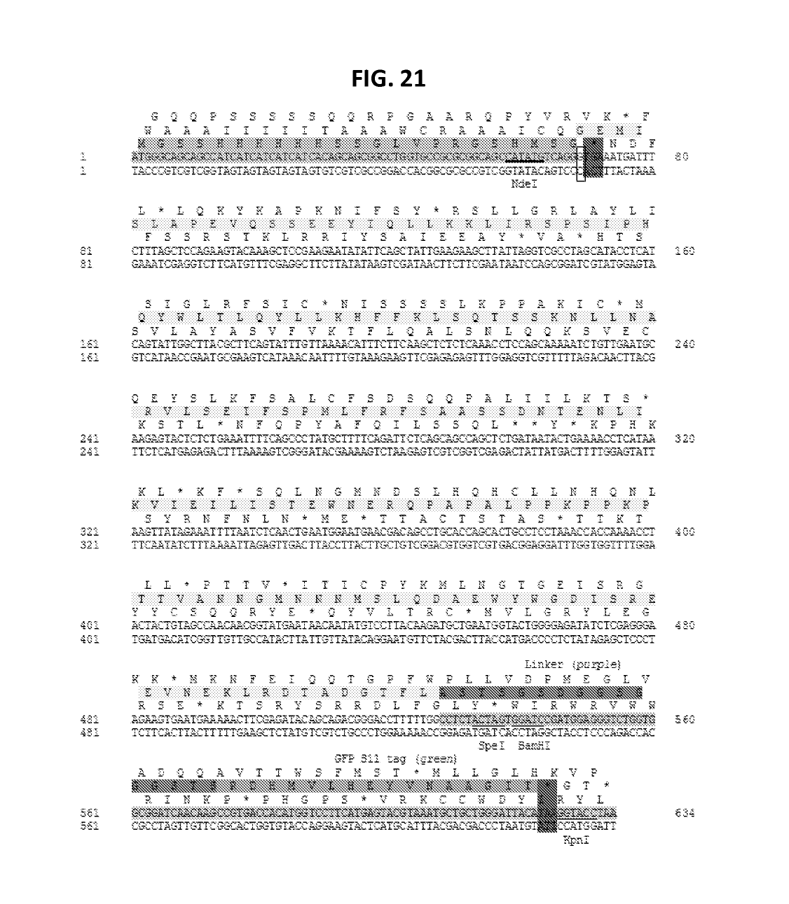

The sequences for BCR domain clones A and F each contained an extra base insertion at the 5′ cloning site that resulted in predicted frame shifts and stop codons in the frames of the fragments (FIGS. 20 and 21). Both clones passed the ORF selection step (described herein) and had detectable Talon® bead-bound fluorescence (FIG. 16B) implying that translation must continue beyond the stop codon and that both ends of the polypeptide are covalently linked. Such artifacts can result from ribosome frame-shift and reinitiation (Adhin and van Duin, J. Mol. Biol., 213:811-818, 1990; Goldman et al., FASEB J., 14:603-611, 2000) and have been reported elsewhere (Goldman et al., FASEB J., 14:603-611, 2000). In the absence of the frame-shift, the calculated mass of clone A is ˜23.4 kDa.

Instead three bands are visible at approximately 16, 18 and 21 kDa (FIG. 16B, middle). This is consistent with the hypothesis that translation reinitiation occurs downstream of the vector Shine-Dalgarno sequence. Talon bound protein for clone F was not detectable by SDS-PAGE (FIG. 16B, middle).

Clone C produced two bands on SDS-PAGE, perhaps because it includes additional ‘linker’ sequence on the ends and might be partially proteolyzed after work-up (Graslund, Nordlund et al. 2008; Graslund, et al. Protein Expr. Purif., 58:210-221, 2008). On the other hand, clone E produced a single, intense band. It has very little ‘additional’ linker sequence on its ends and is the most compact, soluble domain of the set (FIGS. 16a, 16b, 19). Importantly, the ends of fragment E (amino acids 108-300) correspond closely to the construct from which the structure was determined (Musacchio et al., Proc. Natl. Acad. Sci. U.S.A., 93:14373-14378, 1996) (amino acids 105-319). Removing unstructured regions of proteins by engineering the ends of the coding sequence or by proteolysis can improve soluble expression and crystallizability Gao et al., J. Struct. Funct. Genomics, 6:129-134, 2005; Hart and Tarendeau, Acta. Crystallogr. D. Biol. Crystallogr., 62:19-26, 2006; Angelini et al, FEBS J., 276:816-824, 2009).

### Example 3

### A High-Throughput Immobilized Bead Screen for Stable Soluble Proteins and Multi-Protein Complexes Using a Transparent PETE Membranes and a SFP Detector in Talon Agar

This example illustrates growth and induction of bacterial colonies on membranes as in example 1, but a transparent PETE membrane was used in place of the opaque Durapore membrane used in Example 1.

The bead assay plate (a 150 mm diameter Bauer plate) is overlaid with 10 ml of concentrated Split-GFP 1-10 detector (ca. 4 mg per ml, total of 40 mg) and the Split-GFP 1-10 detector is allowed to diffuse into the agarose with gentle rocking (on a rocking table or similar mixer) for 1 hour at 25° C. Subsequently, excess solution is drained off the plate, which is then dried for 5 minutes in a laminar flow hood. The colonies are then partially lysed (e.g., as described in Example 1 above), and the released protein diffuses into the agarose, interacting with the Split-GFP 1-10 detector via the fused Split-GFP S11 tag. Fluorescence is detectable within 1 hour using methods described in Example 1. Bead assay plate is incubated in the cold room overnight, after which immobilized, complemented Split-GFP fluorescence is detected using the methods described above. The soluble proteins bind to the beads, decorating them with fluorescence.

Thus, soluble proteins are identified by speckles (bright beads) under the colony mass. Some Split-GFP 1-10 will diffuse into the colony mass as well, and can stain it will fluorescence. However, this is diffuse (uniform throughout the colony) and so is easily distinguished from the specular, soluble fluorescence bound to beads under the soluble colonies. Colonies corresponding to detected immobilized complemented Split-GFP fluorescence in the bead assay plate can be selected as described above for further study. This application has advantages when it is not feasible to move the colony membrane, as in automated picking operations.

### Example 4

### A High-Throughput Screen for Stable Soluble Proteins Using Biotinylated Agarose-SFP Detector Capture Plates

This example illustrates identification of soluble protein using biotinylated agarose in the hydrogel as a component of the immobilized affinity reagent. Biotinylated agarose is available commercially; for example, from One-Cell Systems, Inc., Cambridge, Mass. The biotinylated agarose is overlaid and incubated with streptavidin solution using standard methods (e.g., 10 ml of 0.1 mg/ml streptavidin in the case of a Bauer plate with 25 ml of agarose) to convert it into biotin-binding agarose (each of the streptavidin has 4 binding sites for biotin, so after binding to the agarose, there are 3 remaining sites). Excess solution is decanted and the plate is overlaid with buffer [What type of Buffer?] and washed to remove unbound streptavidin by incubating with 50 ml of buffer for 1 hour with rocking at room temperature, repeating three times. Next, the plate is overlaid with 10 ml of biotin-GFP 1-10 detector with rocking and incubating as described in Example 3. Unbound excess biotin-GFP 1-10 detector is poured off and the plate is washed 3 times with buffer. The prepared plates are used as in examples 1-3 to assay for soluble protein released (e.g., by partial lysis) from colonies incubated on top of the plates. The colonies express a test protein that is fused to the Split-GFP S11 tag. The Split-GFP S 11-tagged protein will become immobilized in the biotinylated agarose plate through interaction with the immobilized Split-GFP 1-10 detector, which we result in immobilized complemented Split-GFP fluorescence. Thus, detection of immobilized complemented Split-GFP fluorescence in the biotinylated agarose plate indicates that colony immediately above the detected signal expresses a soluble test protein fused to the Split-GFP S11 tag. Additionally, a second, orthogonal tag can be fused to the test protein and detected independently (such as from a different split fluorescent protein, or an antibody tag) to determine if the test protein is full-length or that a soluble protein complexes is present.

It will be understood by those of skill in the art that numerous and various modifications can be made without departing from the spirit of the present disclosure. Therefore, it should be clearly understood that the embodiments disclosed herein are illustrative only.

We claim:

1. A method of identifying a soluble protein, comprising
expressing within at least one host cell a first heterologous amino acid molecule comprising a first test protein;
bringing the at least one host cell into aqueous contact with the surface of a hydrogel comprising an immobilized affinity reagent with affinity for the first heterologous amino acid molecule for a period of time sufficient for transfer of the first amino acid molecule into the hydrogel; and
detecting a complex of the first heterologous amino acid molecule and the immobilized affinity reagent in the hydrogel, wherein the presence of a complex of the first amino acid molecule and the immobilized affinity reagent in the hydrogel identifies the first test protein as a soluble protein.

2. The method of claim 1, wherein the first heterologous amino acid molecule comprises a detection tag that does not bind the immobilized affinity reagent and wherein detecting the complex of the first heterologous amino acid molecule and the immobilized affinity reagent in the hydrogel comprises detecting the presence of the detection tag immobilized in the hydrogel.

3. The method of claim 2, wherein the detection tag is a Split Fluorescent Protein (SFP) tag, wherein the hydrogel comprises a SFP detector, and wherein detecting the presence of the detection tag immobilized in the hydrogel comprises detecting immobilized complemented Split Fluorescent Protein fluorescence in the hydrogel.

4. The method of claim 1, further comprising expressing within the host cell a second heterologous amino acid molecule comprising a SFP detector, wherein the first heterologous amino acid molecule comprises a SFP tag, and wherein detecting the complex of the first heterologous amino acid molecule and the immobilized affinity reagent in the hydrogel comprises detecting immobilized complemented Split Fluorescent Protein fluorescence in the hydrogel.

5. The method of claim 1, further comprising identifying a soluble protein complex, the method further comprising:
expressing within the host cell a second heterologous amino acid molecule comprising a second test protein;
wherein the second heterologous amino acid molecule does not bind to the immobilized affinity reagent;
wherein detecting the complex of the first heterologous amino acid molecule and the immobilized affinity reagent in the hydrogel comprises detecting a complex of the second heterologous amino acid molecule and the immobilized affinity reagent in the hydrogel; and
wherein detecting a complex of the second heterologous amino acid molecule and the immobilized affinity reagent in the hydrogel further identifies the first and second test proteins as a soluble protein complex.

6. The method of claim 5, wherein the second heterologous amino acid molecule comprises a detection tag, and wherein detecting the complex of the second heterologous amino acid molecule and the immobilized affinity reagent in the hydrogel comprises detecting the presence of the detection tag immobilized in the hydrogel.

7. The method of claim 6, wherein the detection tag is a SFP tag, wherein the hydrogel comprises a SFP detector, and wherein detecting the presence of the detection tag immobilized in the hydrogel comprises detecting immobilized complemented Split Fluorescent Protein fluorescence in the hydrogel.

8. The method of claim 5, further comprising expressing within the host cell a third heterologous amino acid molecule comprising a SFP detector, wherein the second heterologous amino acid molecule comprises a second test protein fused to a SFP tag, and wherein detecting the complex of the second heterologous amino acid molecule and the immobilized affinity reagent in the hydrogel comprises detecting immobilized complemented Split Fluorescent Protein fluorescence in the hydrogel.

9. The method of claim 1, wherein each heterologous amino acid comprises a secretion signal sequence.

10. The method of claim 1, further comprising lysing at least one host cell.

11. The method of claim 10, wherein lysing at least one host cell comprises:
contacting at least one host cell with a lysis enzyme;
contacting at least one host cell with a detergent;
subjecting at least one host cell to a freeze thaw cycle; or
a combination of two or more thereof.

12. The method of claim 1, further comprising selecting the host cell that expresses the soluble protein.

13. The method of claim 12, wherein selecting the host cell is performed by a robot.

14. The method of claim 1, wherein the host cell is a bacteria cell.

15. The method of claim 14, wherein the bacteria cell is an E. coli cell.

16. The method of any one of claim 3, 4, 7 or 8, wherein the SFP tag is a Split-Green Fluorescent Protein (GFP)S11 tag and the SFP detector is split-GFP S1-10.

17. The method of any one of claims 1-15, wherein the host cell is separated from the hydrogel by a membrane permeable to soluble protein.

18. The method of claim 17, wherein the membrane is optically translucent.

19. The method of claim 17, wherein the membrane comprises a position locator element.

20. The method of claim 19, wherein the position locator element comprises cells expressing a fluorescent molecule.

21. The method of claim 19, further comprising:
detecting the position locator element;
determining a membrane orientation based on the location of the position locator element; and
selecting the host cell comprising the nucleic acid encoding the soluble test protein based on the membrane orientation.

22. The method of claim 21, wherein the determining and selecting steps are performed by a robot.

23. The method of any one of claims 1-15, wherein the hydrogel comprises agarose.

24. The method of any one of claims 1-15, wherein the hydrogel is contained in a dish or plate.

25. The method of any one of claims 1-15, wherein the first heterologous amino acid molecule further comprises an affinity tag that binds to the immobilized affinity reagent.

26. The method of claim 25, wherein the affinity tag comprises a polyhistidine tag and the immobilized affinity reagent comprises chelated nickel or cobalt.

27. The method of claim 2, wherein the first test protein is fused to an affinity tag that binds to the immobilized affinity reagent and wherein the affinity tag is on the N-terminus of the first test protein and the detection tag is on the C-terminus of the first test protein; or the affinity tag is on the C-terminus of the first test protein and the detection tag is on the N-terminus of the first test protein.

28. The method of any one of claims 4-7, wherein the first heterologous amino acid molecule is encoded by a first nucleic acid molecule that is operably linked to a first promoter and the second heterologous amino acid molecule is encoded by a second nucleic acid molecule that is operably linked to a second promoter.

29. The method of claim 28, wherein the first and second promoters are inducible promoters.

30. The method of claim 29, wherein the first heterologous amino acid molecule comprises a SFP tag, and wherein the second heterologous amino acid molecule comprises a SFP detector, the method further comprising detecting in vivo expression of the first and second amino acid molecules, comprising:
contacting the host cell with a reagent that induces expression from the first promoter for a period of time sufficient to allow expression of the first heterologous amino acid molecule;
contacting the host cell with a reagent that induces expression from the second promoter for a period of time sufficient to allow expression of the second heterologous amino acid molecule; and
detecting complemented Fluorescent Protein fluorescence within the host cell, thereby detecting in vivo expression of the first and second heterologous amino acid molecules.

31. The method of claim 30, wherein contacting the host cell with a reagent that induces expression from the first promoter and contacting the host cell with a reagent that induces expression from the second promoter is performed simultaneously or sequentially.

32. The method of any one of claims 1-4, comprising:
incubating at least two host cells at separately addressable locations on the surface of the hydrogel, wherein each host cell comprises a different first heterologous amino acid molecule comprising a different first test protein.

33. The method of claim 32, wherein the different test proteins are members of a library of test proteins.

34. The method of claim 33, wherein the different test proteins are members of a library of variants of the same protein.

35. The method of any one of claims 5-8, comprising:
incubating at least two host cells at separately addressable locations on the surface of the hydrogel, wherein each host cell comprises a first heterologous amino acid molecule encoding a first test protein and a second heterologous amino acid molecule encoding a second test protein, and wherein the at least two host cells comprise:
a different first heterologous amino acid molecule comprising a different first test protein;
a different second heterologous amino acid molecule comprising a different second test protein; or
a combination thereof.

36. The method of claim 35, wherein the different first test proteins, the different second test proteins or a combination thereof are members of a library of test proteins.

37. The method of claim 36, wherein the different first test proteins, the different second test proteins or a combination thereof are members of a library of variants of the same protein.

38. A kit for performing the method of any one of claims 1-8, comprising:
a nucleic acid construct encoding a SFP tag and a multiple cloning site adjacent thereto, such that an encoding sequence inserted into the multiple cloning site results in a nucleic acid molecule that encodes a test protein fused with the SFP tag;
a hydrogel comprising an immobilized affinity reagent; and
instructions for carrying out the method.

39. A system for detecting a soluble protein, comprising
a first nucleic acid construct encoding a SFP tag and a multiple cloning site adjacent thereto, wherein insertion of a nucleic acid molecule encoding a test protein into the multiple cloning site is expressed as a heterologous amino acid nucleic acid molecule that encodes a heterologous amino acid comprising the test protein and the SFP tag;
a second nucleic acid construct encoding a SFP detector or purified SFP detector, or both;
a host cell comprising the first nucleic acid construct, the second nucleic acid construct, or both; and,
a hydrogel comprising an immobilized affinity reagent with affinity for the first heterologous amino acid molecule.

40. The system of claim 40, wherein the first nucleic acid construct encoding the SFP tag and the multiple cloning site adjacent thereto, further encodes an affinity tag that binds to the immobilized affinity reagent in the hydrogel, such that an encoding sequence inserted into the multiple cloning site results in a nucleic acid molecule that encodes a heterologous amino acid comprising a test protein fused with the SFP tag and the affinity tag.
